(19) United States
(12) Patent Application Publication
(10) Pub. No.: US 2004/0110763 A1
(43) Pub. Date: Jun. 10, 2004

(54) Pyrazolopyridine compound and pharmaceutical use thereof

(76) Inventors: Atsushi Akahane, Osaka (JP); Akira Tanaka, Osaka (JP); Masatoshi Minagawa, Osaka (JP); Hiromichi Itani, Osaka (JP); Hiroaki Ohtake, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/344,894

(22) PCT Filed: Aug. 27, 2001
(86) PCT No.: PCT/JP01/07322

(30) Foreign Application Priority Data
Aug. 28, 2000 (AU) PQ9698

(51) Int. Cl. A61K 31/501

(52) U.S. Cl. 514/252.04

(57) ABSTRACT

A pyrazolopyridine compound of formula (I) wherein: R¹ is hydrogen, lower alkyl optionally substituted by susbtituent(s), or cyclo(lower)alkyl which may be interrupted by an oxygen or nitrogen atom and optionally substituted by substituent(s); R² is hydrogen, halogen or lower alkoxy; R³ is a substituent; and n is an integer from 1 to 4, provided R³ may be different from each other when n is 2, 3 or 4, or a salt thereof. The pyrazolopyridine compound (I) and salt thereof of the present invention are adnosine antagonists and are useful for the prevention and/or treatment of depression, dementia (e.g. Alzheimer's disease, cerebrovascular dementia, dementia accompanying Parkinson's disease, etc.) Parkinson's disease, anxiety, pain, cerebrovascular disease (e.g. stroke, etc.), heart failure and the like.

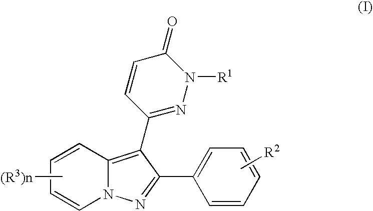

### TECHNICAL FIELD

The present invention relates to a novel pyrazolopyridine compound and a salt thereof, which are useful as medicaments.

### BACKGROUND ART

Some pyrazolopyridine compounds to be useful as psychostimulant, remedy for renal failure, or the like are known (e.g. EP-0299209, EP-0379979, EP-0467248, EP-0516941, etc.).

### DISCLOSURE OF INVENTION

The present invention relates to a novel pyrazolopyridine compound and a pharmaceutically acceptable salt thereof, which are useful as medicaments, whose toxicity may be reduced as compared with the known pyrazolopyridine compounds; processes for the preparation of said pyrazolopyridine compound and a salt thereof; a pharmaceutical composition comprising, as an active ingredient, said pyrazolopyridine compound or a pharmaceutically acceptable salt thereof; a use of said pyrazolopyridine compound or a pharmaceutically acceptable salt thereof as a medicament; and a method for using said pyrazolopyridine compound or a pharmaceutically acceptable salt thereof for therapeutic purposes, which comprises administering said pyrazolopyridine compound or a pharmaceutically acceptable salt thereof to a human being or an animal.

The pyrazolopyridine compound and a salt thereof are adenosine antagonists (especially, A₁ receptor and A₂ (particularly A₂ₐ) receptor dual antagonists) and possess various pharmacological actions such as anticatalepsy action, cognitive enhancing action, analgesic action, locomotor action, antidepressant action, diuretic action, cardioprotective action, cardiotonic action, vasodilating action (e.g. cerebral vasodilating action, etc.), the action of increasing the renal blood flow, renal protective action, improvement action of renal function, enhancing action of lipolysis, inhibition action of anaphylactic bronchoconstriction, acceleration action of the insulin release, the action of increasing the production of erythropoietin, inhibiting action of platelet aggregation, or the like.

They are useful as cognitive enhancer, antianxietry drug, antidementia drug, psychostimulant, analgesic, cardioprotective agent, antidepressant, ameliorants of cerebral circulation, tranquilizer, drug for heart failure, cardiotonic agent, antihypertensive agent, drug for renal failure (renal insufficiency), drug for renal toxicity, renal protective agent, drug for improvement of renal function, diuretic, drug for edema, antiobesity, antiasthmatic, bronchodilator, drug for apnea, drug for gout, drug for hyperuricemia, drug for sudden infant death syndrome (SIDS), ameliorants of immunosuppressive action of adenosine, antidiabetic agent, drug for ulcer, drug for pancreatitis, drug for Meniere's syndrome, drug for anemia, drug for thrombosis, drug for myocardial infarction, drug for obstruction, drug for arteriosclerosis obliterans, drug for thrombophlebitis, drug for cerebral infarction, drug for transient ischemic attack, drug for angina pectoris, or the like;

and useful for the prevention and/or treatment of a disease resulting from a stimulation of adenosine A₁ and/or A₂ receptor, such as depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure, hypertension (e.g. essential hypertension, nephrogenous hypertension, etc.),

circulatory insufficiency (acute circulatory insufficiency) cuased by

for example, ischemia/reperfusion injury (e.g. myocardial ischemia/reperfusion injury, cerebral ischemia/reperfusion injury, peripheral ischemia/reperfusion injury, etc.), shock (e.g. endotoxin shock, hemorrhagic shock, etc.), surgical procedure, or the like,

post-resuscitation asystole, bradyarrhythmia, electro-mechanical dissociation, hemodynamic collapse, SIRS (systemic inflammatory response syndrome), multiple organ failure, renal failure (renal insufficiency) (e.g. acute renal failure, etc.), renal toxicity [e.g. renal toxicity induced by a drug such as cisplatins, gentamicin, FR-900506 (disclosed in EP-0184162), cyclosporin (e.g. cyclosporin A) or the like; glycerol, etc.], nephrosis, nephritis, edema (e.g. cardiac edema, nephrotic edema, hepatic edema, idiopathic edema, drug edema, acute angioneurotic edema, hereditary angioneurotic edema, carcinomatous ascites, gestational edema, etc.),

obesity, bronchial asthma, gout, hyperuricemia, sudden infant death syndrome, immunosuppression, diabetes, ulcer such as peptic ulcer (e.g. gastric ulcer, duodenal ulcer, etc.), pancreatitis, Meniere's syndrome, anemia, dialysis-induced hypotension, constipation, ischemic bowel disease, ileus (e.g. mechanical ileus, adynamic ileus, etc.), and

myocardial infarction, thrombosis (e.g. arterial thrombosis, cerebral thrombosis, etc.), obstruction, arteriosclerosis obliterans, thrombophlebitis, cerebral infarction, transient ischemic attack, angina pectoris, or the like,

in which the preferred one may be Parkinson's disease and symptoms associating therewith, depression, dementia (e.g. Alzheimer's disease, cerebrovascular dementia, dementia accompanying Parkinson's disease, etc.), anxiety, pain, cerebrovascular disease (e.g. stroke, etc.), Meniere's syndrome or cerebral infarction.

The novel pyrazolopyridine compound of the present invention can be shown by the following formula (I).

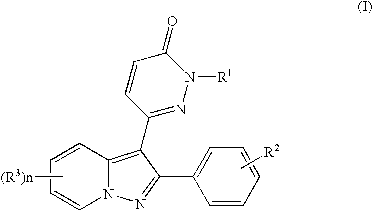

wherein

R¹ is hydrogen, lower alkyl optionally substituted by suitable substituenr(s), or cyclo(lower)alkyl which may be interrupted by an oxygen or nitrogen atom and optionally substituted by suitable substituenr(s);

R² is hydrogen, halogen or lower alkoxy;

R³ is a substituent; and

n is an integer from 1 to 4, provided R³ may be different with each other when n is 2, 3 or 4,

or a salt thereof.

The object compound (I) and a salt thereof of the present invention can be prepared by the following processes.

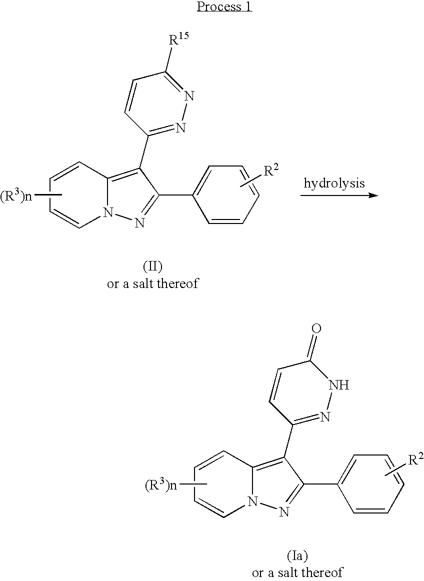

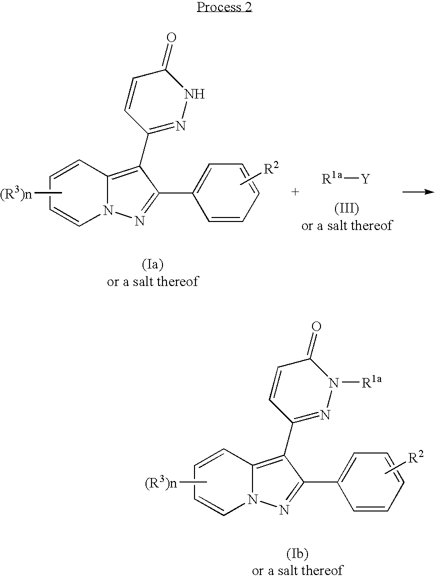

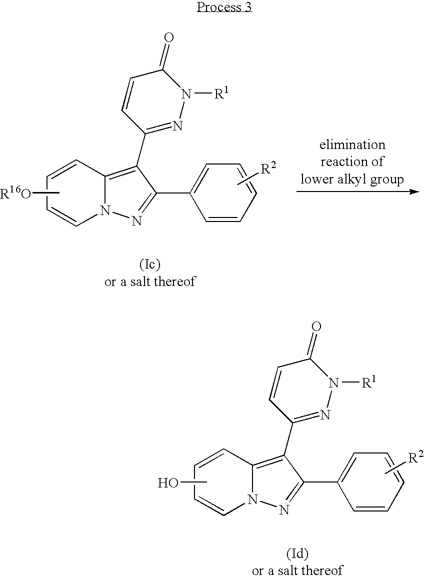

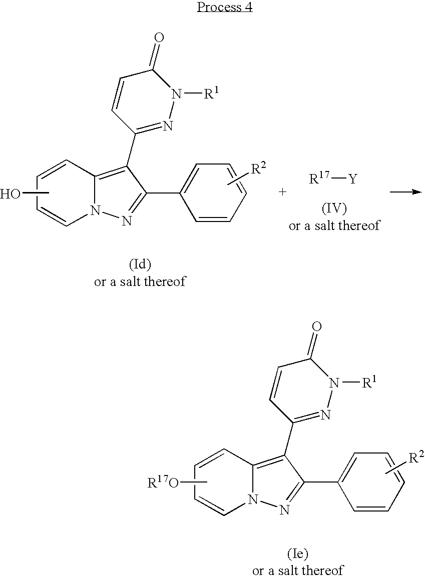

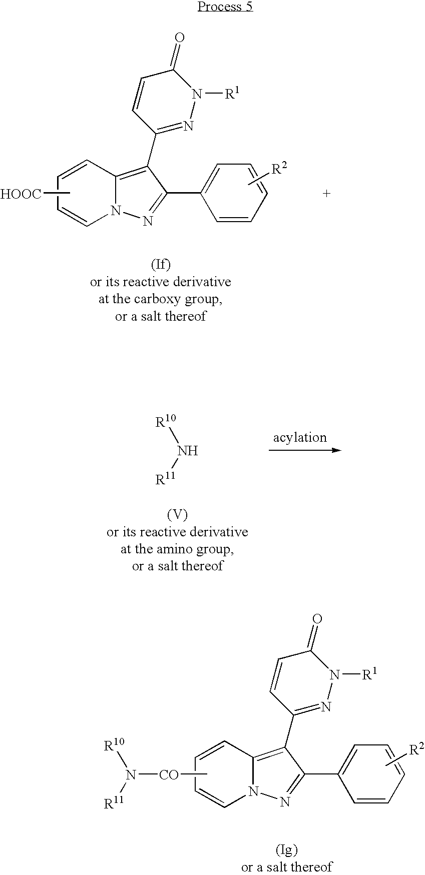

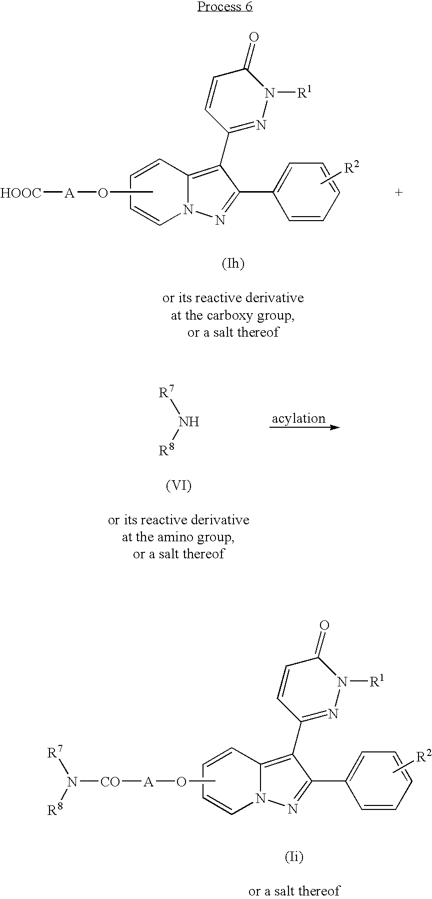

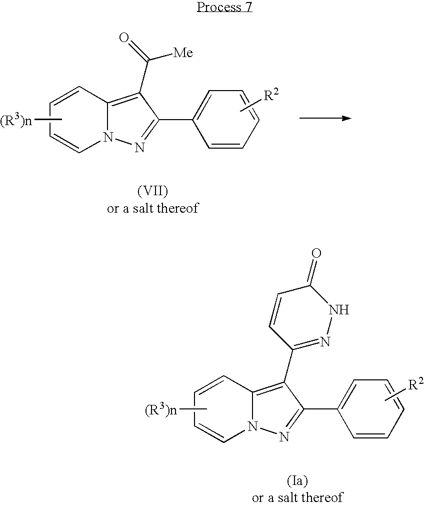

wherein R¹, R², R³ and n are as defined above,

R¹⁵ is arylsulfonyl optionally substituted by suitable substituent(s),

di(lower)alkylamino,

lower alkoxy,

lower alkylthio,

or acyloxy;

R^{1a} is lower alkyl or cyclo (lower) alkyl which may be interrupted by an oxygen atom;

R¹⁶ is lower alkyl;

R¹⁷ is a substituent other than hydrogen, selected from among -A-R⁴ and —R⁹,

[in which, A is lower alkylene,

R⁴ is hydrogen;

cyclo(lower)alkyl;

aryl optionally substituted by lower alkoxy;

a group of the formula:

R⁵—(R⁶—)N—

wherein R⁵ and R⁶ are each independently

hydrogen, or

lower alkyl;

heterocyclic group optionally substituted by

oxo, lower alkyl or

lower alkoxy(lower)alkyl;

carboxy;

lower alkoxycarbonyl;

aryl(lower)alkoxycarbonyl;

lower alkanoyl;

a group of the formula:

R⁷—(R⁸—)N—CO—

wherein R⁷ and R⁸ are each independently

hydrogen;

lower alkyl optionally substituted by lower alkoxy, N,N-di(lower)alkylamino or heterocyclic group;

cyclo(lower)alkyl optionally substituted by hydroxy;

aryl optionally substituted by lower alkoxy; or

a group of the formula:

Het-CO—

wherein Het is N-containing heterocyclic group optionally substituted by

lower alkyl, lower alkanoyl, lower alkoxycarbonyl, N,N-di(lower)alkylcarbamoyl or aryl(lower)alkyl,

R⁹ is hydrogen;

aryl optionally substituted by lower alkanoylamino;

heterocyclic group optionally substituted by

lower alkyl, lower alkanoyl, lower alkoxycarbonyl, carbamoyl, N,N-di(lower)alkylcarbamoyl, aryl(lower)alkyl, lower alkoxy, halo(lower)alkyl or nitro; or

arylsulfonyl optionally substituted by

lower alkyl or lower alkoxy],

R¹⁰ and R¹¹ are each independently

hydrogen;

cyclo (lower) alkyl;

heterocyclic group optionally substituted by lower alkyl;

lower alkyl optionally substituted by hydroxy, lower alkoxy, aryl, aryloxy, N,N-di(lower)alkylamino or heterocyclic group,

R¹⁰ and R¹ may be combined together with N atom to which they are attached to form N-containing heterocyclic group optionally substituted by lower alkyl, aryl, lower alkanoyl or heterocyclic group;

R⁷ and R⁸ are each independently hydrogen;

lower alkyl optionally substituted by lower alkoxy, di(lower)alkylamino or heterocyclic group;

cyclo(lower)alkyl optionally substituted by hydroxy;

aryl optionally substituted by lower alkoxy; and

Y is a leaving group.

The starting compound(II) or a salt thereof is novel and can be prepared, for example, by the following reaction schemes.

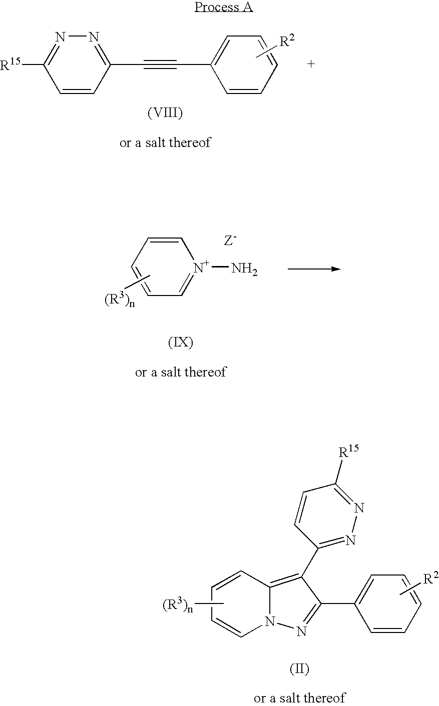

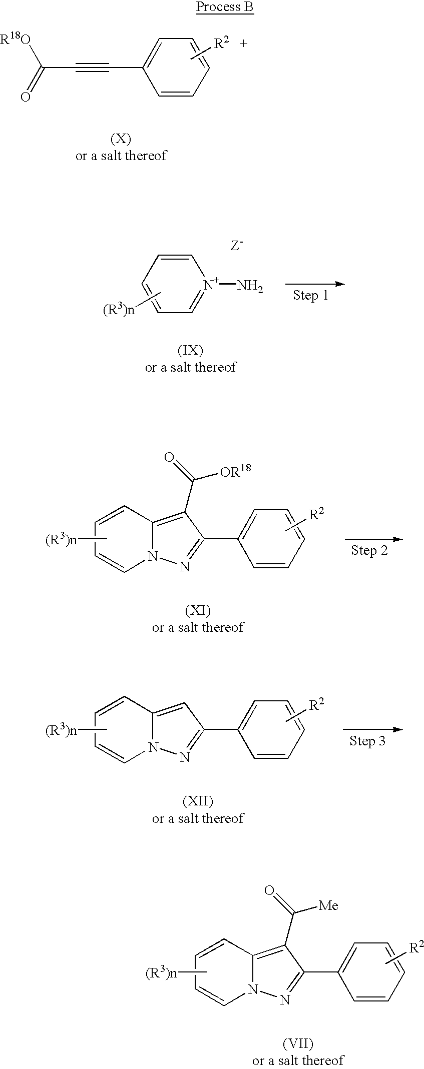

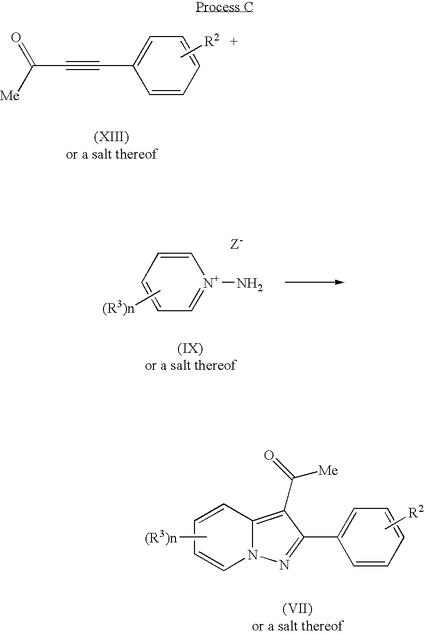

wherein R², R³, R¹⁵ and n are as defined above,

R¹⁸ is lower alkyl,

Z⁻ is an anion.

In addition to the processes as mentioned above, the object compound (I) and a salt thereof can be prepared, for example, according to the procedures as illustrated in Examples in the present specification or in a manner similar thereto.

The starting compounds can be prepared, for example, according to the procedures as illustrated in Preparations in the present specification or in a manner similar thereto.

The object compound (I) and a salt thereof can be prepared according to the methods as shown in a Preparation or Examples, or in a manner similar thereto.

It is to be noted that the object compound (I) may include the geometrical isomer(s) due to the double bond(s) and/or the stereo isomer(s) due to the asymmetric carbon atom(s). In this regard, one isomer can be converted to another according to a conventional method in this field of the art.

It is also to be noted that the solvating form of the compound (I) (e.g. hydrate, etc.) and any form of the crystal of the compound (I) are included within the scope of the present invention.

It is further to be noted that the object compound (I) may include the dimer, which is coupling through urea, of the formula (Ij)

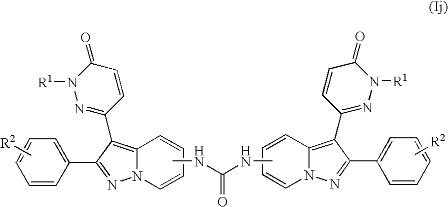

wherein

R¹ and R² are as defined above, or a salt thereof,

Suitable salts of the object compound (I) are conventional pharmaceutically acceptable ones and include a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), an organic acid salt (e.g. acetate, trifluoroacetate, maleate, tartrate, fumarate, methanesulfonate, benzenesulfonate, formate, toluenesulfonate, etc.), an inorganic acid salt (e.g. hydrochloride, hydrobromide, hydriodide, sulfate, phosphate, etc.), a salt with an amino acid (e.g. arginine, aspartic acid, glutamic acid, etc.), and the like.

Suitable examples and illustrations of the various definitions which the present invention includes within the scope thereof and which appear in the above and following description in the present specification are explained in detail as follows.

The term “lower” is intended to mean 1 to 6 carbon atom(s) unless otherwise indicated.

Suitable “lower alkyl” may include straight or branched ones such as methyl, ethyl, propyl, isopropyl, butyl, tert-butyl, pentyl, hexyl or the like, in which the preferred one may be (C1-C4)alkyl and the more preferred one may be methyl, ethyl, propyl or isopropyl.

Suitable “lower alkylene” may include straight or branched ones such as methylene, ethylene, propylene, isopropylene, butylene, tert-butylene, pentylene, hexylene or the like, in which the preferred one may be (C1-C5)alkylene and the more preferred one may be methylene, ethylene or propylene.

Suitable “lower alkynyl” may include straight or branched ones such as ethynyl, 1-propynyl, 1-methylethynyl, 2-butynyl, 2-methyl-3-butynyl, 2-pentynyl, 1-hexynyl or the like, in which the preferred one may be (C.sub.2-C.sub.4)alkynyl and the more preferred one may be ethynyl.

Suitable “cyclo (lower) alkyl” may be cyclo (C3-C8)-alkyl such as cyclopropyl, cyclobutyl, cyclopentyl, cyclohexyl, cycloheptyl, cyclooctyl or the like, in which the preferred one may be cyclo(C3-C7)alkyl such as cyclopropyl, cyclopentyl, cyclohexyl or cycloheptyl.

Suitable “lower alkoxy” may include straight or branched ones such as methoxy, ethoxy, propoxy, isopropoxy, butoxy, tert-butoxy, pentyloxy, hexyloxy or the like, in which the preferred one may be (C1-C4)alkoxy and the more preferred one may be methoxy.

Suitable “aryl” may be phenyl, naphthyl and the lile, in which the preferred one may be (C6-C10)aryl and the most preferred one may be phenyl.

Suitable “aryl (lower) alkyl” may include phenyl (lower) alkyl (e.g. benzyl, phenethyl, etc.), diphenyl(lower)alkyl (e.g. benzhydryl, etc.) or triphenyl (lower) alkyl (e.g. trityl, etc.) and the like, in which the preferred one may be (C6-C10)aryl(lower)alkyl, and the more preferred one may be phenyl(C1-C4)alkyl.

Suitable “arylsulfonyl” may include phenylsulfonyl, tolylsulfonyl, naphthylsulfonyl and the like, and said “arylsulfonyl” may have one or more (preferably 1 to 3) suitable substituent(s) such as aforesaid lower alkoxy, aforesaid halogen, or the like.

Suitable “lower alkylsulfonyl” may be methylsulfonyl, ethylsufonyl, propylsulfonyl, butylsulfonyl, t-butylsulfonyl, pentylsulfonyl, hexylsufonyl, in which the preferred one may be (C1-C4)alkylsulfonyl and the most preferred one may be methylsufonyl.

Suitable “halogen” may be fluoro, chloro, bromo and iodo.

Suitable “heterocyclic group” may be saturated or unsaturated monocyclic or polycyclic heterocyclic groups containing at least one hetero atom selected from among oxygen, sulfur and nitrogen.

The particularly preferred example of said heterocyclic group may include unsaturated 3- through 8-membered heteromonocyclic groups containing 1 through 4 nitrogen atom(s), such as pyrrolyl, pyrrolinyl, imidazolyl, pyrazolyl, pyridyl and its N-oxide, pyrimidyl, pyrazinyl, pyridazinyl, triazolyl (e.g. 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), dihydrotriazinyl (e.g. 4,5-dihydro-1,2,4-triazinyl, 2,5-dihydro-1,2,4-triazinyl, etc.), etc.;

3- through 8-membered saturated heteromonocyclic groups containing 1 through 4 nitrogen atom(s), such as pyrrolidinyl, imidazolidinyl, piperidyl (e.g. piperidino, etc.), piperazinyl, etc.;

unsaturated condensed heterocyclic groups containing 1 through 5 nitrogen atom(s), such as indolyl, isoindolyl, indolizinyl, benzimidazolyl, quinolyl, isoquinolyl, indazolyl, benzotriazolyl, tetrazolopyridyl, tetrazolopyridazinyl (e.g. tetrazolo [1,5-b]pyridazinyl etc.), dihydrotriazolopyridazinyl, etc.;

3- through 8-membered unsaturated heteromonocyclic groups containing 1 or 2 oxygen atoms and 1 through 3 nitrogen atom(s), such as oxazolyl, isoxazolyl, oxadiazolyl (e.g. 1,2,4-oxadiazolyl, 1,3,4-oxadiazolyl, 1,2,5-oxadiazolyl, etc.), etc.;

3- through 8-membered saturated heteromonocyclic groups containing 1 or 2 oxygen atom(s) and 1 through 3 nitrogen atoms, such as morpholinyl, oxazolidinyl (e.g. 1,3-oxazolidinyl etc.), etc.;

unsaturated condensed heterocyclic groups containing 1 or 2 oxygen atom(s) and 1 through 3 nitrogen atom(s), such-as benzoxazolyl, benzoxadiazolyl, etc.;

3- through 8-membered unsaturated heteromonocyclic groups containing 1 or 2 sulfur atom(s) and 1 through 3 nitrogen atom(s), such as 1,3-thiazolyl, 1,2-thiazolyl, thiazolinyl, thiadiazolyl (e.g. 1,2,4-thiadiazolyl, 1,3,4-thiadiazolyl, 1,2,5-thiadiazolyl, 1,2,3-thiadiazolyl), etc.;

3- through 8-membered saturated heteromonocyclic groups containing 1 or 2 sulfur atom(s) and 1 through 3 nitrogen atom(s), such as thiazolidinyl etc.;

3- through 8-membered unsaturated heteromonocyclic groups containing 1 sulfur atom, such as thienyl etc.;

unsaturated condensed heterocyclic groups containing 1 or 2 sulfur atoms and 1 through 3 nitrogen atom(s), such as benzothiazolyl, benzothiadiazolyl, etc.;

3- through 8-membered unsaturated heteromonocyclic groups containing 1 or 2 oxygen atom(s), such as furyl, pyranyl, dioxolyl, etc.;

3- through 8-membered saturated heteromonocyclic groups containing 1 or 2 oxygen atom(s), such as oxolanyl, tetrahydropyranyl (e.g. tetrahydro-2H-pyran-2-yl etc.), dioxolanyl, etc.; and

unsaturated condensed heterocyclic groups containing 1 or 2 oxygen atom(s), such as isobenzofuranyl, chromenyl (e.g. 2H-chromen-3-yl etc.), dihydrochromenyl (e.g. 3,4-dihydro-2H-chromen-4-yl etc.), etc.

Suitable “N-containing heterocyclic group” may be aforesaid “heterocyclic group”, in which said group contains at least one N atom in its ring members.

Suitable “an acyl group” may include lower alkanoyl, carboxy, protected carboxy, and the like.

Suitable examples of aforesaid “lower alkanoyl” maybe formyl, acetyl, propionyl, butyryl, isobutyryl, pivaloyl, hexanoyl, or the like, in which the preferred one may be (C1-C4)alkanoyl and the more preferred one may be formyl and acetyl.

Suitable examples of aforesaid “protected carboxy” may be

i) esterified carboxy, in which suitable esterified carboxy may include lower alkoxycarbonyl (e.g. methoxycarbonyl, ethoxycarbonyl, propoxycarbonyl, butoxycarbonyl, t-butoxycarbonyl, pentyloxycarbonyl, hexyloxycarbonyl, etc.), aryl(lower)alkoxycarbonyl (e.g. benzyloxycarbonyl, phenethyloxycarbonyl, 2-phenylpropoxycarbonyl, 4-phenylbutoxycarbonyl, 4-phenylpentyloxycarbonyl, 1,3-diphenylhexyloxycarbonyl, etc.), and the like;

ii) amidated carboxy, in which suitable amidated carboxy may include carbamoyl, N-(lower)alkylcarbamoyl (e.g. N-methylcarbamoyl, N-ethylcarbamoyl, N-isopropylcarbamoyl, N-butylcarbamoyl, N-pentylcarbamoyl, N-hexylcarbamoyl, etc.),

N,N-di(lower)alkylcarbamoyl [e.g. N,N-dimethylcarbamoyl, N,N-diethylcarbamoyl, N-methyl-N-ethylcarbamoyl, N,N-dipropylcarbamoyl, N,N-di(t-butyl)carbamoyl,

N-pentyl-N-hexylcarbamoyl, etc.],

N-lower alkyl-N-ar(lower)alkylcarbamoyl (e.g. N-methyl-N-benzylcarbamoyl, etc), and the like.

Suitable “a leaving group” may include halogen as mentioned above, hydroxy, acyloxy such as alkanoyloxy (e.g. acetoxy, propionyloxy, etc.), sulfonyloxy (e.g. mesyloxy, tosyloxy, etc.), and the like.

Suitable “anion” may be formate, acetate, trifluoroacetate, maleate, tartrate, methanesulfonate, benzenesulfonate, toluenesulfonate, chloride, bromide, iodide, sulfate, phosphate, or the like.

The preferred embodiment of the compound (I) is explained as follows.

1. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is a group of the formula:

R⁴-A-O—

in which

A is lower alkylene, and

R⁴ is hydrogen;

cyclo(lower)alkyl;

aryl optionally substituted by lower alkoxy;

a group of the formula:

R⁵—(R⁶—)N—

wherein R⁵ and R⁶ are each independently

hydrogen, or

lower alkyl;

heterocyclic group optionally substituted by

oxo, lower alkyl or

lower alkoxy(lower)alkyl;

carboxy;

lower alkoxycarbonyl;

aryl(lower)alkoxycarbonyl;

lower alkanoyl;

a group of the formula:

R⁷— (R⁸—)N—CO—

wherein R⁷ and RB are each independently

hydrogen;

lower alkyl optionally substituted by

lower alkoxy, N,N-di(lower)alkylamino or

heterocyclic group;

cyclo(lower)alkyl optionally substituted by hydroxy;

aryl optionally substituted by lower alkoxy;

a group of the formula:

Het-CO—

wherein Het is N-containing heterocyclic group optionally substituted by

lower alkyl, lower alkanoyl, lower alkoxycarbonyl, N,N-di(lower)alkylcarbamoyl, or

aryl(lower)alkyl; or

halogen.

2. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is a group of the formula:

R⁹—O—

in which

R⁹ is hydrogen;

aryl optionally substituted by lower alkanoylamino;

heterocyclic group optionally substituted by

lower alkyl, lower alkanoyl, lower alkoxycarbonyl, carbamoyl, N,N-di(lower)alkylcarbamoyl, aryl(lower)alkyl, lower alkoxy, halo(lower)alkyl or nitro;

arylsulfonyl optionally substituted by

lower alkyl or lower alkoxy.

3. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is a group of the formula:

R¹⁰—N(—R¹¹)—CO—

in which

R¹⁰ and R¹¹ are each independently

hydrogen;

cyclo(lower)alkyl optionally substituted by hydroxy;

heterocyclic group optionally substituted by lower alkyl lower alkoxycarbonyl,

aryl optionally substituted by

halogen, or aryl(lower)alkyl,

lower alkoxy, hydroxy, halogen, or halo(lower)alkyl;

lower alkyl optionally substituted by

hydroxy, lower alkoxy, lower alkylthio,

aryl optionally substituted by lower alkyl, lower alkoxy, hydroxy or halogen,

aryloxy, lower alkoxycarbonylamino,

N,N-di(lower)alkylamino, or

heterocyclic group optinally substituted by halogen or hydroxy;

lower alkenyl; or

aryl optionally substituted by

lower alkyl, hydroxy(lower) alkyl, halo (loer) alkyl, lower alkoxy,

aryloxy optionally substituted by lower alkyl or halogen,

hydroxy, halogen, lower alkanoyl, amino, lower alkanoylamino, N,N-di(lower)alkylamino, aryl(lower)alkanoyl, cyano or nitro;

R¹⁰ and R¹¹ may be combined together with N atom to which they are attached to form N-containing heterocyclic group optionally substituted by

lower alkyl optionally substituted by lower alkylamino,

aryl optionally substituted by lower alkoxycarbonyl or lower alkoxy,

lower alkanoyl, heterocyclic group,

hydroxy (lower) alkyl, lower alkylsulfonylamino, amino,

oxo, nitro, lower alkoxy(lower)alkyl, lower alkoxycarbonyl, N-lower alkylcarbamoyl,

cyclo (lower) alkyl, aryl (lower) alkoxy or lower alkoxy.

4. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is a group of the formula:

R¹²—N(—R¹³)—

in which

R¹² and R¹³ are each independently

hydrogen;

lower alkyl optionally substituted by lower alkoxy;

lower alkanoyl optionally substituted by aryl or halogen;

lower alkoxycarbonyl;

lower alkylsulfonyl; or

R¹² and R¹³ may be combined together with N atom to which they are attached to form N-containing heterocyclic group optionally substituted by

hydroxy, oxo, lower alkyl, lower alkoxy,

lower alkanoyl optionally substituted by

N,N-di(lower)alkylamino or aryl,

lower alkoxycarbonyl,

N,N-di(lower)alkylcarbamoyl,

lower alkylsulfonyl, arylsulfonyl, aryl,

aryl(lower)alkyl or heterocyclic group.

5. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is a group of the formula:

R¹⁴-A′-

in which

A′ is lower alkynyl,

R¹⁴ is hydroxy; cyclo(lower)alkyl; or aryl.

6. The compound (I), wherein

R¹ is hydrogen, lower alkyl optionally substituted by lower alkoxy, or cyclo(lower)alkyl which may be interrupted by an oxygen or nitrogen atom and optionally substituted by lower alkyl;

R² is hydrogen, halogen or lower alkoxy;

R³ is carboxy, lower alkoxycarbonyl or cyano.

The more preferred embodiment of the compound (I) is explained as follows.

1. The compound (I), wherein

R¹ is hydrogen, lower alkyl optionally substituted by lower alkoxy, tetrahydrofuryl, tetrahydropyranyl or piperidinyl;

R² is hydrogen, halogen or lower alkoxy;

R³ is a group of the formula:

R⁴-A-O—

in which

A is lower alkylene, and

R⁴ is hydrogen;

cyclo(lower)alkyl;

phenyl optionally substituted by lower alkoxy;

a group of the formula:

R⁵—(R⁶—)N—

wherein R⁵ and R⁶ are each independently

hydrogen or lower alkyl;

aziridinyl, pyrrolidinyl, piperidinyl, morpholinyl, pyridyl or isoindolyl, each of which is optionally substituted by

oxo, lower alkyl or lower alkoxy(lower)alkyl;

carboxy;

lower alkoxycarbonyl;

phenyl(lower)alkoxycarbonyl;

lower alkanoyl;

a group of the formula:

R⁷—(R⁸—)N—CO—

wherein R⁷ and R⁸ are each independently

hydrogen;

lower alkyl optionally substituted by

lower alkoxy, N,N-di(lower) alkylamino or pyridyl;

cyclo(lower)alkyl optionally substituted by hydroxy;

phenyl optionally substituted by lower alkoxy; or

a group of the formula:

Het-CO—

wherein Het is pyrrolidinyl, piperidinyl, piperazinyl or morpholinyl, each of which is optionally substituted by

lower alkyl, lower alkanoyl, lower alkoxycarbonyl, N,N-di(lower)alkylcarbamoyl, phenyl(lower)alkyl,

2. The compound (I), wherein

R¹ is lower alkyl;

R² is hydrogen;

R³ is a group of the formula:

R⁹—O—

in which

R⁹ is hydrogen;

phenyl optionally substituted by lower alkanoylamino;

piperidinyl, tetrahydropyranyl or pyridinyl, each of which is optionally substituted by

lower alkyl, lower alkanoyl, lower alkoxycarbonyl, carbamoyl, N,N-di(lower)alkylcarbamoyl, phenyl(lower)alkyl, lower alkoxy, halo(lower)alkyl or nitro;

phenylsulfonyl optionally substituted by lower alkyl or lower alkoxy.

3. The compound (I), wherein

R¹ is lower alkyl;

R² is hydrogen;

R³ is a group of the formula:

R¹⁰—N(—R¹¹)—CO—

in which

R¹⁰ and R¹¹ are each independently

hydrogen;

cyclo(lower)alkyl;

thiazolyl optionally substituted by lower alkyl;

lower alkyl optionally substituted by

hydroxy, lower alkoxy, phenyl, phenoxy, N,N-di (lower) alkylamino, pyrrolidinyl or pyridinyl; or

R¹⁰ and R¹¹ may be combined together with N atom to which they are attached to form pyrrolidinyl, piperidinyl, hexahydroazepinyl, piperazinyl or morpholinyl, each of which is optionally substituted by

lower alkyl, phenyl, lower alkanoyl or pyridinyl.

4. The compound (I), wherein

R¹ is hydrogen or lower alkyl;

R² is hydrogen;

R³ is a group of the formula:

R¹²—N(—R¹³)—

in which

R¹² and R¹³ are each independently

hydrogen;

lower alkyl optionally substituted by lower alkoxy;

lower alkanoyl optionally substituted by phenyl or halogen;

lower alkoxycarbonyl;

lower alkylsulfonyl; or

R¹² and R¹³ may be combined together with N atom to which they are attached to form pyrrolidinyl, piperidinyl, piperazinyl or morpholinyl, each of which is optionally substituted by

hydroxy, oxo, lower alkyl, lower alkoxy,

lower alkanoyl optionally substituted by

N,N-di(lower)alkylamino or phenyl,

lower alkoxycarbonyl,

N,N-di(lower)alkylcarbamoyl

lower alkylsulfonyl, phenylsulfonyl, phenyl, phenyl(lower)alkyl, pyridinyl or pyrimidinyl.

5. The compound (I), wherein

R¹ is lower alkyl;

R² is hydrogen;

R³ is a group of the formula:

R¹⁴-A′-

in which

A′ is lower alkynyl,

R¹⁴ is hydroxy; cyclo(lower)alkyl; or phenyl.

6. The compound (I), wherein

R¹ is lower alkyl;

R² is hydrogen;

R³ is carboxy, lower alkoxycarbonyl or cyano.

The processes for preparing the object pyrazolopyridine compound(I) are explained in detail in the following.

Process 1

The compound (Ia) or a salt thereof can be prepared by subjecting the compound (II) or a salt thereof to hydrolysis.

Suitable salt of the compound (II) can be referred to an acid addition salt as exemplified for the compound (I).

This reaction is carried out in accordance with a conventional method.

The hydrolysis is preferably carried out in the presence of a base or an acid including Lewis acid.

Suitable base includes an inorganic base and an organic base such as an alkali metal (e.g. sodium, potassium, etc.), an alkaline earth metal (e.g. magnesium, calcium, etc.), the hydroxide or carbonate or hydrogencarbonate thereof, trialkylamide (e.g. trimethylamine, triethylamine, etc.), hydrazine, picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid includes an organic acid (e.g. formic acid, acetic acid, propionic acid, trichloroacetic acid, trifluoroacetic acid, etc.) and an inorganic acid (e.g. hydrochloric acid, hydrobromic acid, sulfuric acid, hydrogen chloride, hydrogen bromide, etc.).

The elimination using Lewis acid such as trihaloacetic acid (e.g. trichloroacetic acid, trifluoroacetic acid, etc.) or the like is preferably carried out in the presence of cation trapping agents (e.g. anisole, phenol, etc.).

The reaction is usually carried out in a solvent such as water, an alcohol (e.g. methanol, ethanol, isopropyl alcohol, etc.), tetrahydrofuran, dioxane, toluene, methylene chloride, ethylene dichloride, chloroform, N,N-dimethylformamide, N,N-dimethylacetamide, or any other organic solvents which do not adversely affect the reaction, or a mixture thereof.

A liquid base or acid can be also used as the solvent. The reaction temperature is not critical and the reaction is usually carried out under cooling to heating.

Process 2

The compound (Ib) or a salt thereof can be prepared by reacting the compound (Ia) or a salt thereof with the compound (III) or a salt thereof.

Suitable salt of the compound (Ia) can be referred to an acid addition salt as exemplified for the compound (I).

Suitable salt of the compound (III) can be referred to the ones as exemplified for the compound (I).

The present reaction may be carried out in a solvent such as water, phosphate buffer, acetone, chloroform, acetonitrile, nitrobenzene, methylene chloride, ethylene chloride, formamide, N,N-dimethylformamide, methanol, ethanol, sec-butanol, amyl alcohol, diethyl ether, dioxane, tetrahydrofuran, dimethyl sulfoxide, or any other organic solvent which does not adversely affect the reaction, preferably in ones having strong polarities. Among the solvents, hydrophilic solvents may be used in a mixture with water. When the compound (III) is in liquid, it can also be used as a solvent. The reaction is preferably conducted in the presence of a base, for example, inorganic base such as alkali metal hydroxide, alkali metal carbonate, alkali metal bicarbonate, alkali metal hydride (e.g. sodium hydride, etc.), organic base such as trialkylamine, and the like.

The reaction temperature is not critical, and the reaction is usually carried out at ambient temperature, under warming or under heating.

The present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) or the like.

When Y is —OH, activation of OH with triphenylphosphine and the like may be necessary.

Process 3

The compound (Id) or a salt thereof can be prepared by subjecting the compound (Ic) or a salt thereof to elimination reaction of alkyl group.

Suitable salts of the compound (Ic) and (Id) can be referred to the ones as exemplified for the compound (I).

This reaction is carried out in accordance with a conventional method such as hydrolysis.

The hydrolysis is preferably carried out in the presence of a base or an acid including Lewis acid.

Suitable base includes an inorganic base and an organic base such as an alkali metal (e.g. sodium, potassium, etc.), an alkaline earth metal (e.g. magnesium, calcium, etc.), hydroxide or carbonate or bicarbonate thereof, trialkylamine (e.g. trimethylamine, triethylamine, etc.), hydrazine, picoline, 1,5-diazabicyclo[4.3.0]non-5-ene, 1,14-diazabicyclo[2.2.2]octane, 1,8-diazabicyclo[5.4.0]undec-7-ene, or the like.

Suitable acid includes an organic acid (e.g. formic acid, acetic acid, propionic acid, trichloroacetic acid, trifluoroacetic acid, etc.) and an inorganic acid (e.g. hydrochloric acid, hydrobromic acid, sulfuric acid, hydrogen chloride, hydrogen bromide, etc.).

The elimination using Lewis acid (e.g. aluminium chloride, titanium trichloride, tin tetrachloride, etc.) or the like is preferably carried out in the presence of cation trapping agents (e.g. anisole, phenol, etc.).

The reaction is usually carried out in a solvent such as water, alcohol (e.g. methanol, ethanol, isopropyl alcohol, etc.), tetrahydrofuran, dioxane, toluene, methylene chloride, ethylene dichloride, chloroform, N,N-dimethylformamide, N,N-dimethylacetamide, or any other organic solvents which do not adversely affect the reaction, or a mixture thereof. A liquid base or acid can be also used as the solvent.

The reaction of this process can be also carried out according to a conventional reduction method employed in this field of the art (e.g. chemical reduction, catalytic reduction, etc.).

The reaction temperature is not critical and the reaction is usually carried out at ambient temperature, under warming or under heating.

Process 4

The compound (Ie) or a salt thereof can be prepared by reacting the compound (Id) or a salt thereof with the compound (IV) or a salt thereof.

Suitable salt of the compound (Id), (IV) and (Ie) can be referred to the ones as exemplified for the compound (I).

The reaction of this process can be carried out in the manner similar to that of Process 2.

Process 5

The compound (Ig) or a salt thereof can be prepared by reacting the compound (If) or its reactive derivative at the carboxy group, or a salt thereof with the compound (V) or its reactive derivative or a salt thereof.

Suitable reactive derivative of the compound (V) may include Schiff's base type imino or its tautomeric enamine type isomer formed by the reaction of the compound (V) with a carbonyl compound such as aldehyde, ketone or the like; a silyl derivative formed by the reaction of the compound (V) with a silyl compound such as N,O-bis(trimethylsilyl)acetamide, N-trimethylsilylacetamide or the like; a derivative formed by the reaction of the compound (V) with phosphorus trichloride or phosgene, and the like.

Suitable reactive derivative of the compound (If) may include an acid halide, an acid anhydride, an activated ester, and the like. The suitable example may be an acid chloride; acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anhydride;

an activated amide with imidazole, 4-substituted imidazole, dimethylpyrazole, triazole or tetrazole; an activated ester (e.g., cyanomethyl ester, methoxymethyl ester, dimethyliminomethyl [(CH₃)₂N⁺═CH—] ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenylthio ester, p-nitrophenyl thioester, p-cresyl thioester, carboxymethyl thioester, pyranyl ester, pyridyl ester, piperidyl ester, 8-quinolyl thioester, etc.); an ester with a N-hydroxy compound (e.g., N,N-dimethylhydroxylamine, 1-hydroxy-2-(1H)-pyridone, N-hydroxysuccinimide, N-hydroxybenzotriazole (HOBt), N-hydroxyphthalimide, 1-hydroxy-6-chloro-1H-benzotriazole, etc.); and the like. These reactive derivatives can optionally be selected from them according to the kind of the compound (If) to be used.

The reaction is usually carried out in a conventional solvent such as water, acetone, dioxane, acetonitrile, chloroform, methylene chloride, ethylene chloride, tetrahydrofuran, ethyl acetate, N,N-dimethylformamide (DMF), pyridine or any other organic solvents which do not adversely affect the reaction, or the mixture thereof.

When the compound (If) is used in free acid form or its salt form in the reaction, the reaction is preferable carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diisopropylcarbodiimide; N-ethyl-N′-(3-dimethylaminopropyl)carbodiimide (EDAC); N,N-carbonyl-bis(2-methylimidazole); pentamethyleneketene-N-cyclohexylimine; diphenylketene-N-cyclohexylimine; ethoxyacetylene; 1-alkoxy-1-chloroethylene; trialkyl phosphite; isopropyl polyphosphate; phosphorous oxychloride (phosphoryl chloride); phosphorous trichloride; thionyl chloride; oxalyl chloride; triphenylphosphite; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorous oxychloride, etc.; or the like.

The reaction may be also be carried out in the presence of an organic or inorganic base such as an alkali metal bicarbonate, tri(lower)alkylamine, pyridine, N-(lower)-alkylmorphorine, N,N-di(lower)alkylbenzylamine, or the like.

The reaction temperature is not critical, and the reaction is usually carried out under cooling to heating.

Process 6

The compound (Ii) or a salt thereof can be prepared by reacting the compound (Ih) or its reactive derivative at the carboxy group, or a salt thereof with the compound (VI) or its reactive derivative or a salt thereof.

This reaction can be carried out in the same manner as in the aforementioned Process 5, and therefore the reagents to be used and the reaction conditions (e.g., solvent, reaction temperature, etc.) can be referred to those of Process 5.

Process 7

The object compound (Ia) or a salt thereof can be prepared by subjecting the compound (VII) or a salt thereof to formation reaction of pyridazinone ring.

Suitable salts of the compounds (Ia) and (VII) can be referred to acid addition salts as exemplified for the compound (I).

The formation reaction of this process can be carried out, for example, by reacting the compound (VII) or a salt thereof with glyoxylic acid or its reactive derivative or a salt thereof and hydrazine or a salt thereof.

Suitable salt of glyoxylic acid can be referred to a salt with a base as exemplified for the compound (I).

Suitable salt of hydrazine can be referred to an acid addition salt as exemplified for the compound (I).

Suitable reactive derivative of glyoxylic acid may be the ones conventionally used in this field of the art such as an activated ester thereof.

The reaction can be carried out in the presence or absence of a solvent.

The reaction temperature is not critical and the reaction is usually carried out under warming to heating.

Process A

The compound (II) or a salt thereof can be prepared by reacting the compound (VIII) or a salt thereof with the compound (IX) or a salt thereof.

Suitable salts of the compounds (II), (VIII) and (IX) can be referred to acid addition salts as exemplified for the compound (I).

The reaction is usually carried out in a solvent such as water, methylene chloride, ethylene chloride, N,N-dimethylformamide or any other solvent which does not adversely influence the reaction or a mixture thereof.

The reaction can be carried out in the presence of a base such as alkalimetal carbonate (e.g. sodium carbonate, potassium carbonate, etc.), alkali metal hydroxide (e.g. sodium hydroxide, potassium hydroxide, etc.), ar(lower)alkyltri(lower)alkylammonium halide (e.g. benzyltrimethylammonium chloride, etc.) or the like.

The reaction temperature is not critical and the reaction is usually carried out under cooling, at room temperature or under warming.

Process B

Step 1

The compound (XI) or a salt thereof can be prepared by reacting the compound (X) or a salt thereof with the compound (IX) or a salt thereof.

This reaction can be carried out in the same manner as in the aforementioned Process A, and therefore the reagents to be used and the reaction conditions (e.g., solvent, reaction temperature, etc.) can be referred to those of Process A.

Step 2

The deesterification reaction of this step can be carried out by the methods disclosed in Preparation 3 mentioned later or the similar manners thereto.

Step 3

The introduction reaction of acetyl group of this step can be carried out by the methods disclosed in Preparation 4 mentioned later or the similar manners thereto.

Process C

The compound (VII) or a salt thereof can be prepared by reacting the compound (XIII) or a salt thereof with the compound (IX) or a salt thereof.

This reaction can be carried out in the same manner as in the aforementioned Process A, and therefore the reagents to be used and the reaction conditions (e.g., solvent, reaction temperature, etc.) can be referred to those of Process A.

The object compound (I) of the present invention is an adenosine antagonist and possesses the various pharmacological actions as stated before.

In order to show the usefulness of the compound (I) of the present invention, the pharmacological test result of the representative compound of the present invention is shown in the following.

Test 1: Adenosine Antagonistic Activity

[I] Test Method

The adenosine antagonistic activity [Ki(nM)] of the test compound was examined by radioligand binding techniques using 8-cyclopentyl-1,3-dipropylxanthine, [dipropyl-2,3-³H(N)] ([³H]DPCPX, 4.5 nM) for human A₁ receptor and [³H]CGS 21680 (20 nM) for human A₂ₐ receptor.

[II] Test Compound

5-Methoxy-3-(3-oxo-2-isopropyl-2,3-dihydropyridazin-6-yl)-2-phenylpyrazolo[1,5-a]pyridine (Example 2)

5-Hydroxy-3-(3-oxo-2-isopropyl-2,3-dihydropyridazin-6-yl)-2-phenylpyrazolo[1,5-a]pyridine (Example 3)

5-(2-Dimethylamino)ethoxy-3-(3-oxo-2-isopropyl-2,3-dihydropyridazin-6-yl)-2-phenylpyrazolo[1,5-a]pyridine (Example 4)

5-(2-pyridinyloxy)-3-(3-oxo-2-isopropyl-2,3-dihydropyridazin-6-yl)-2-phenylpyrazolo[1,5-a]pyridine (Example 31)

N,N-dimethyl-3-(3-oxo-2-isopropyl-2,3-dihydropyridazin-6-yl)-2-phenylpyrazolo[1,5-a]pyridine-5-carboxamide (Example 72)

5-(4-methyl-1-piperazinyl)-3-(3-oxo-2-isopropyl-2,3-dihydropyridazin-6-yl)-2-phenylpyrazolo[1,5-a]pyridine (Example 96)

tert-butyl 3-(3-oxo-2-isopropyl-2,3-dihydropyridazin-6-yl)-2-phenylpyrazolo[1,5-a]pyridin-5-ylcarbamate (Example 145)

[III] Test Result

TABLE 1

Adenosine
receptor
binding (Ki:nM)

| Test compound (Example No.) | A₁ | A₂ₐ |
|---|---|---|
| 2 | 0.15 | 1.38 |
| 3 | 0.14 | 1.05 |
| 4 | 0.98 | 2.35 |
| 31 | 0.42 | 1.44 |
| 72 | 0.25 | 1.68 |
| 96 | 0.24 | 1.28 |
| 145 | 0.48 | 1.03 |

Test 2: Anticatalepsy Activity in Mouse

[I] Test Method

The test compound (3.2 mg/kg) was administered orally with ddY mice (n=7). Then, haloperidol (0.32 mg/kg) was injected intraperitoneally 30 min. after the administration of the compound. Thirty min. after the injection, the cataleptic responses of mice were measured. The forelimbs of each mouse were placed on a 3 cm high, 3 mm wide horizontal bar, and the duration of cataleptic posture was measured for up to 30 sec.

[II] Test Compound

5-Methoxy-3-(3-oxo-2-isopropyl-2,3-dihydropyridazin-6-yl)-2-phenylpyrazolo[1,5-a]pyridine (Example 2)

5-(2-Dimethylamino)ethoxy-3-(3-oxo-2-isopropyl-2,3-dihydropyridazin-6-yl)-2-phenylpyrazolo[1,5-a]pyridine (Example 4)

5-(2-pyridinyloxy)-3-(3-oxo-2-isopropyl-2,3-dihydropyridazin-6-yl)-2-phenylpyrazolo[1,5-a]pyridine (Example 31)

N,N-dimethyl-3-(3-oxo-2-isopropyl-2,3-dihydropyridazin-6-yl)-2-phenylpyrazolo [1,5-a]pyridine-5-carboxamide (Example 72)

5-(4-methyl-1-piperazinyl)-3-(3-oxo-2-isopropyl-2,3-dihydropyridazin-6-yl)-2-phenylpyrazolo [1,5-a]pyridine (Example 96)

tert-butyl 3-(3-oxo-2-isopropyl-2,3-dihydropyridazin-6-yl)-2-phenylpyrazolo[1,5-a]pyridin-5-ylcarbamate (Example 145)

[III] Test Result

TABLE 2

| Test compound | Manifestation rate of catalepsy |
|---|---|
| (Example No.) | (number of mouse) |
| 2 | 0/7 |
| 4 | 0/7 |
| 31 | 0/7 |
| 72 | 0/7 |
| 96 | 0/7 |
| 145 | 0/7 |

The pyrazolopyridine compound (I) and a salt thereof of this invention are useful as adenosine antagonists (especially, A₁ receptor and A₂ (particularly A₂ₐ) receptor dual antagonists) and for the prevention and/or the treatment of depression, dementia (e.g. Alzheimer's disease, cerebrovascular dementia, dementia accompanying Parkinson's disease, etc.), Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack, angina pectoris, and the like.

The pharmaceutical composition of this invention can be used in the form of a pharmaceutical preparation, for example, in a solid, semisolid or liquid form, which contains the pyrazolo-pyridine compound (I) or a pharmaceutically acceptable salt thereof as an active ingredient in admixture with an organic or inorganic carrier or excipient suitable for rectal, pulmonary (nasal or buccal inhalation), nasal, ocular, external (topical), oral or parenteral (including subcutaneous, intravenous and intramuscular) administrations or insufflation. The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. In addition, auxiliary, stabilizing agents, thickening agents, coloring agents and perfumes may be used where necessary. The pyrazolopyridine compound (I) or a pharmaceutically acceptable salt thereof is included in a pharmaceutical composition in an amount sufficient to produce the desired aforesaid pharmaceutical effect upon the process or condition of diseases.

For applying the composition to a human being or an animal, it is preferable to apply it by intravenous, intramuscular, pulmonary or oral administration, or insufflation. While the dosage of therapeutically effective amount of the pyrazolo-pyridine compound (I) varies depending on the age and condition of each individual patient to be treated, in the case of intravenous administration, a daily dose of 0.01-100 mg of the pyrazolo-pyridine compound (I) per kg weight of a human being or an animal, in the case of intramuscular administration, a daily dose of 0.1-100 mg of the pyrazolopyridine compound (I) per kg weight of a human being or an animal, and in case of oral administration, a daily dose of 0.5-100 mg of the pyrazolopyridine compound (I) per kg weight of a human being or an animal is generally given for the prevention and/or treatment of the aforesaid diseases.

The following Preparation and Examples are given for the purpose of illustrating the present invention in more detail.

Preparation 1

To a stirred mixture of 1-amino-4-methoxypyridinium iodide (400 mg) and 3-benzenesulfonyl-6-phenylethynylpyridazine (250 mg) in N,N-dimethylformamide (10 ml) was added powder potassium carbonate (650 mg) at ambient temperature. After being stirred at ambient temperature for 18 hours, the mixture was poured into water. The resultant precipitate was collected by filtration to give 3-(3-phenylsulfonylpyridazin-6-yl)-5-methoxy-2-phenylpyrazolo[1,5-a]pyridine (100 mg).

mp: 203.5-205.5° C.(AcOEt)

IR (nujol):1644, 1540, 1523, 1332 cm⁻¹

NMR (DMSO-d6, δ): 3.91(3H,s), 6.81(1H,dd,J=2.8, 7.5 Hz), 7.36-7.62(7H,m), 7.62-7.89(4H,m), 8.08(1H,d,J=6.7 Hz), 8.19(1H,d,J=9.1 Hz), 8.76(1H,d,J=7.5 Hz)

APCI/MS: 443[M+H]⁺

Anal.Calcd for C₂₄H₁₈N₄O₃S: C, 65.14; H, 4.10; N, 12.66 Found: C, 64.77; H, 4.18; N, 12.37

Preparation 2

To a stirred mixture of 1-amino-4-methoxypyridinium iodide (91.1 g) and ethyl phenylpropiolate (18.0 g) in N,N-dimethylformamide (180 ml) was added potassium carbonate (57.1 g) at ambient temperature. After being stirred at ambient temperature for 18 hours, the mixture was poured into water. The mixture was extracted with ethyl acetate. The organic layer was washed with water and brine, and dried over magnesium sulfate, then evaporated in vacuo. The residue was purified by silica-gel column chromatography (ethylacetate:n-hexane=1:4) to give ethyl 5-methoxy-2-phenylpyrazolo[1,5-a]pyridine-3-carboxylate (24.7 g).

IR (nujol): 1714, 1643, 1546, 1513, 1417, 1299, 1226, 1201, 1174, 1133 cm⁻¹

NMR (DMSO-d6, δ): 1.23(3H,t,J=1.2 Hz), 3.93(3H,s), 4.20(2H,q,J=7.1 Hz), 6.86(1H,dd,J=2.8, 7.5 Hz), 7.39-7.53(4H,m), 7.63-7.80(2H,m), 8.74(1H,d,J=7.5 Hz) APCI/MS: 297[M+H]⁺

Preparation 3

To a stirred mixture of ethyl 5-methoxy-2-phenylpyrazolo[1,5-a]pyridine-3-carboxylate (32.0 g) in methanol (160 ml) was added 10% aqueous sodium hydroxide (86.4 ml) at ambient temperature. After being stirred at 75° C. for 3 hours, the reaction mixture was cooled to room temperature, and the solvent methanol was evaporated. To the residue was added N,N-dimethylformamide (160 ml) and the mixture was acidified with 6N-hydrochloric acid (45 ml). After being stirred at 90° C. for 1 hour, the reaction mixture was poured into ice-water (300 ml). After being stirred at 0° C. for 1 hour, the resultant precipitate was collected by filtration to give 5-methoxy-2-phenylpyrazolo[1,5-a]pyridine (23.63 g).

IR (nujol): 1644, 1565, 1536, 1265, 1228 cm⁻¹

NMR (DMSO-d6, δ): 3.84(3H,s), 6.56(1H,dd,J=2.7, 7.6 Hz), 6.83(1H,s), 7.01(1H,d,J=2.6 Hz), ^{70.30}-7.55(3H,m), 7.90-8.03(2H,m), 8.54(1H,d,J=7.6 Hz)

APCI/MS: 225[M+H]⁺

Preparation 4

A mixture of 5-methoxy-2-phenylpyrazolo[1,5-a]pyridine (1.50 g), acetic anhydride (0.76 ml), methanesulfonic acid (22 μl), and nitrobenzene (6.00 ml) was stirred at 125° C. for 6 hours. Then to the reaction mixture was added methanol (1.50 ml) and 10% aqueous sodium hydroxide (19.5 ml) at 0° C. The mixture was extracted with dichloromethane. The organic layer was washed with water and brine, and dried over magnesium sulfate, then evaporated in vacuo. The residue was purified by silica-gel column chromatography (dichloromethane: ethylacetate=1:0-50:1-30:1) to give 1-(5-methoxy-2-phenylpyrazolo[1,5-a]pyridin-3-yl)ethanone (1.23 g).

IR (nujol): 1646, 1617, 1508, 1267, 1236 cm⁻¹

NMR (DMSO-d6, δ): 2.02(3H,s), 3.93(3H,s), 6.89 (1H,dd,J=2.7, 7.5 Hz), 7.42-7.68(7H,m), 8.73(1H,d,J=7.5 Hz)

APCI/MS: 267 [M+H]⁺

Preparation 5

To a stirred solution of 4-phenyl-3-butyn-2-one (4.2 g) and 1-amino-4-methoxypyridinium iodide (16.2 g) in N,N-dimethylformamide (110 ml) was added powder potassium carbonate (16.1 g) at ambient temperature. After stirring for 14 hours, water was added to the mixture. The resultant precipitate was collected by filtration to give 1-(5-methoxy-2-phenylpyrazolo[1,5-a]pyridin-3-yl)ethanone (6.78 g).

NMR(DMSO-d6, δ): 2.20(3H,s), 3.93(3H,s), 6.89 (1H,dd,J=2.8 Hz, 7.5 Hz), 7.49-7.64(6H,m), 8.73(1H,d,J=7.5 Hz)

APCI/MS: 267[M+H]⁺

Preparation 6

The solution of (2R,6S)-2,6-dimethylmorpholine (1.61 ml), 2-bromoethanol (1.36 ml), KHCO₃(2.87 g) in CH₃CN (7 ml) was stirred at 70° C. for 3 hours. After being cooled, the mixture was filtered, and washed with CH₂Cl₂, then evaporated. The residue was purified by silica-gel (55 g) column chlomathography (CHCl₃:MeOH=9:1) to give 2-((2R,6S)-2,6-dimethylmorpholinyl)ethanol (1.94 g).

NMR(CDCl₃, δ): 1.18(6H,d,J=6.3 Hz), 1.93(2H,t,J=10.9 Hz), 2.61(2H,t, J=5.3 Hz), 2.86(2H,td,J=1.6, 10.5 Hz), 3.12(1H,br,s), 3.69(2H,t,J=5.3 Hz), 3.70-3.90(2H,m)

APCI/MS: 160[M+H]⁺

Preparation 7

To the solution of 2-((2R,6S)-2,6-dimethylmorpholinyl) ethanol (1.0 g) in Toluene (5 ml) was added the solution of SOCl₂ (0.596 ml) in Toluene (1 ml) at 0° C., and the mixture was stirred for 1 hour at 70° C. After being cool, to the mixture was added IPE, and stirred for 20 minutes at 0° C. The resultant precipitate was collected by filtration, and dried in vacuo for 6 hours at ambient temperature to give (2R,6S)-4-(2-chloroethyl)-2,6-dimethylmorpholine hydrochloride (0.86 g).

NMR (DMSO-d6,δ): 1.12(6H,d,J=6.3 Hz), 2.60-2.80(2H,m), 3.44-3.50(4H,m), 3.95-4.10(4H,m), 11.47(1H,br,s)

APCI/MS: 178[M−HCl+H]⁺

Preparation 8

To the solution of 1-(2-hydroxyethyl)-2-pyrrolidinone (3.0 g) in Toluene (15 ml) was added the solution of SOCl₂(2.2 ml) in Toluene (3 ml) at 0° C., and the mixture was stirred for 1 hour at 60° C. To the mixture was added saturated sodium hydrogen carbonate solution and the mixture was stirred for 30 minutes, and separated. The water layer was extracted with AcOEt, then the organic layer was washed with saturated sodium hydrogen carbonate solution, water and brine, and dried over magnesium sulfate, then evaporated in vacuo. The residue was purified by silica-gel (90 g) column chlomathography (AcOEt:n-hexane=1:1-1:0) to give 1-(2-chloroethyl)-2-pyrrolidinone (2.75 g).

NMR(CDCl₃, δ): 1.95-2.20(2H,m), 2.41(2H,t,J=8.0 Hz), 3.53(2H,t,J=7.0 Hz), 3.55-3.80(4H,m)

APCI/MS: 148[M+H]⁺

5-Methoxy-3-(3-phenylsulfonylpyridazin-6-yl)-2-(2-fluorophenyl)pyrazolo[1,5-a]pyridine was prepared by similar procedure as that of Preparation 1.

NMR(DMSO-d6, δ): 3.93(3H,s), 6.89(1H,dd,J=7.5, 2.8 Hz), 7.30-7.51 (3H,m), 7.56-7.84(6H,m), 8.06(2H,d,J=8.0 Hz), 8.24(1H,d, J=9.1 Hz), 8.79(1H,d,J=7.5 Hz)

APCI/MS: 461 [M+H]⁺

5-Methoxy-3-(3-phenylsulfonylpyridazin-6-yl)-2-(4-fluorophenyl)pyrazolo[1,5-a]pyridine was prepared by similar procedure as that of Preparation 1.

NMR(DMSO-d6, δ): 3.91(3H,s), 6.86(1H,dd,J=7.5, 2.7 Hz), 7.33(2H, t,J=8.8 Hz), 7.49(1H,d,J=9.1 Hz), 7.57-7.81(6H,m), 8.08(2H,d, J=7.7 Hz), 8.21(1H,d,J=9.1 Hz), 8.76(1H,d,J=7.5 Hz)

APCI/MS: 461 [M+H]⁺

Preparation 11

To a solution of 3-(6-methoxy-3-pyridazinyl)-2-phenylpyrazolo[1,5-a]pyridine (0.5 g) in tetrahydrofuran (25 ml) was added dropwise 1.56M n-butyllithium in n-hexane (1.27 ml) below −70° C. under nitrogen atmosphere. After stirring at −70° C. for 5 minutes, a piece of dryice was added to the mixture, which was stirred for 0.5 hours under the same conditions. Evaporation the solvent gave a residue, which was dissolved in water and acidified with 1N—HCl. The resultant precipitate was collected by filtration, which was dissolved in chloroform. The organic layer was washed with brine, dried over magnesium sulfate and evaporated in vacuo. The residue was recrystallized from a mixture of AcOEt and n-hexane to give 3-(6-methoxy-3-pyridazinyl)-2-phenylpyrazolo[1,5-a]pyridine-7-carboxylic acid (0.46 g) as a solid.

mp: 181-182° C.

NMR(DMSO-d6, δ): 4.09(3H,s), 7.18(1H,d,J=4.8 Hz), 7.25(1H,d, J=4.8 Hz), 7.47-7.65(7H,m), 8.22-8.25(1H,m), 14.27(1H,s)

ESI/MS: 345 [M−H]⁺

Preparation 12

A mixture of 3-(3-methoxy-6-pyridazinyl)-2-phenylpyrazolo[1,5-a]pyridine-7-carboxylic acid (247 mg), triethylamine (0.189 ml), and diphenylphosphoryl azide (0.292 ml) in t-BuOH (4.3 ml) was stirred at 80° C. for 16 hours. After cooling to room temperature, the mixture was partitioned between AcOEt and water. The organic layer washed with water and brine, dried over magnesium sulfate and evaporated in vacuo. The residue was purified by silica gel column chromatography (CHCl₃-AcOEt, 97:3 elution) to give tert-butyl 3-(3-methoxy-6-pyridazinyl)-2-phenylpyrazolo[1,5-a]pyridin-7-ylcarbamate (293 mg)

mp: 178-179° C.

NMR(DMSO-d6, δ): 1.54(9H,s), 4.08(3H,s), 7.15(1H,d,J=9.2 Hz), 7.28(1H,d,J=9.2 Hz), 7.39-7.48(5H,m), 7.60-7.73(2H,m), 7.73-7.75(1H,m), 9.33(1H,s)

ESI/MS: 418 [M+H]⁺

Preparation 13

To a solution of tert-butyl 3-(3-methoxy-6-pyridazinyl)-2-phenylpyrazolo[1,5-a]pyridin-7-ylcarbamate in dioxane (2 ml) was added 4N—HCl in dioxane (2 ml) at ambient temperature. After stirring for 1 hour, the mixture was partitioned between an aqueous sodium bicarbonate and chloroform. The organic layer was dried over magnesium sulfate and evaporated in vacuo. The residue was recrystallized from a mixture of AcOEt and n-hexane to give 7-amino-3-(3-methoxy-6-pyridazinyl)-2-phenylpyrazolo[1,5-a]pyridine (111 mg) as a solid.

mp: 235-238° C.

NMR(DMSO-d6, δ): 4.05(3H,s), 6.18-6.21(1H,m), 6.88(2H,s), 7.09(1H,d,J=4.6 Hz), 7.21(1H,d,J=4.6 Hz), 7.27-7.31(2H,m), 7.44-7.47(3H,m), 7.56-7.59(2H,m)

ESI/MS: 318 [M+H]⁺

Preparation 14

A mixture of 2-iodo-5-methoxypyridine (81.5 g), ethynylbenzene (45.7 ml), (Ph₃P)₂PdCl₂ (12.2 g), CuI (3.3 g) and triethylamine (145 ml) in THF (800 ml) was stirred at reflux for 8 hours. After being cool, the reaction mixture was filtered and evaporated. The residue was purified by silica-gel (600 g) column chromatography (n-Hexane:AcOEt=4:1→1:1). The residue was purified again by silica-gel (550 g) column chromatography (CH₂Cl₂ only→CHCl₃ only→CHCl₃: AcOEt=9:1) to give 5-methoxy-2-(phenylethynyl)pyridine (38.72 g).

NMR(DMSO-d6, δ): 3.91(3H, s), 7.30-7.50(4H, m), 7.50-7.65(3H, m), 8.19(1H,d, J=8.2 Hz)

ESI/MS: 210[M+H]⁺

Preparation 15

A mixture of 5-methoxy-2-(phenylethynyl)pyridine (40 g) and O-(2,4-dinitrophenyl)hydroxylamine (76.1 g) in dioxane (800 ml) was stirred at 90° C. for 5.5 hours. After being cooled, K₂CO₃ (52.8 g) and DMF (400 ml) was added, and the mixture was stirred at ambient temperature for 5 hours. The reaction mixture was poured into water (2 l), and extracted with AcOEt (2 1×2). The organic layer was washed with water (1 l×4), 1N—HCl (500 ml), water (500 ml), saturated sodium hydrogen carbonate solution (1 l), water (1 l), and brine (1 l), dried over magnesium sulfate, filtered, and evaporated in vacuo. The residue was purified by silica-gel (750 g) column chromatography (n-Hexane: AcOEt=4:1) to give 6-methoxy-2-phenylpyrazolo[1,5-a]pyridine (16.58 g).

ESI/MS: 225[M+H]⁺

Preparation 16

A mixture of 6-methoxy-2-phenylpyrazolo [1,5-a]pyridine (15 g), MSOH (0.217 ml) and Ac₂O (88.4 ml) was stirred at 125° C. for 7 hours. To the reaction mixture were added MeOH (54 ml) and aq.NH₃ at 0° C., and the mixture was extracted three times with AcOEt (250 ml). The organic layer was combined and washed with water (300 ml) and brine (100 ml), and was dried over magnesium sulfate, and evaporated. The residue was purified by silica-gel (400 g) column chromatography (n-Hexane:AcOEt=4:1→1:1) to give 3-acetyl-6-methoxy-2-phenylpyrazolo[1,5-a]pyridine (8.70 g).

ESI/MS: 289[M+Na]⁺

Preparation 17

7-Methoxy-3-(3-phenylsulfonylpyridazin-6-yl)-2-phenylpyrazolo[1,5-a]pyridine was prepared by similar procedure as that of Preparation 1.

NMR(DMSO-d6, δ): 4.16(3H,s), 6.68(1H,d,J=7.3 Hz), 7.35-7.62(5H, m), 7.62-7.90(5H,m), 7.92(1H,d,J=8.5 Hz), 8.00-8.15(2H,m), 8.22 (1H,d,J=9.1 Hz)

APCI/MS: 443[M+H]⁺

4-Methoxy-3-(3-phenylsulfonylpyridazin-6-yl)-2-phenylpyrazolo[1,5-a]pyridine was prepared by similar procedure as that of Preparation 1.

NMR (DMSO-d6, δ): 3.74(3H,s), 6.84(1H,d,J=7.7 Hz), 7.01(1H,t, J=7.3 Hz), 7.16-7.45(5H,m), 7.62-7.90(3H,m), 7.95-8.14(2H,m), 8.17(1H,d,J=8.8 Hz), 8.46(1H,d,J=8.8 Hz), 8.49(1H,d,J=6.5 Hz)

APCI/MS: 443[M+H]⁺

### EXAMPLE 1

A mixture of 3-(3-phenylsulfonylpyridazin-6-yl)-5-methoxy-2-phenylpyrazolo[1,5-a]pyridine (50.0 mg), sodium hydroxide (500 mg), water (2.0 ml), and dioxane (5.0 ml) was refluxed for 5 hours. The reaction mixture was acidified with 1N-hydrochloric acid. The mixture was extracted with ethyl acetate. The organic layer was washed with aqueous sodium hydrogen carbonate and brine, and dried over magnesium sulfate, then evaporated in vacuo. The residue was purified by silica-gel column chromatography (chloroform:methanol=9:1) to give 5-methoxy-3-(3-oxo-2,3-dihydropyridazin-6-yl)-2-phenylpyrazolo[1,5-a]-pyridine (40.0 mg).

mp: 245.5-247° C. (AcOEt)

IR (nujol): 1679, 1652, 1575, 1560, 1540, 1513 cm⁻¹

NMR (DMSO-d6, δ): 3.86(3H,s), 6.74(1H,dd,J=2.8, 7.5 Hz), 6.81(1H,d,J=9.8 Hz), 7.08(1H,d,J=9.8 Hz), 7.16(1H,d,J=2.8 Hz), 7.36-7.64(5H,m), 8.67(1H,d,J=7.5 Hz), 13.07(1H,s)

APCI/MS: 319[M+H]⁺

Anal.Calcd for C₁₈H₁₄N₄O₂: C, 67.91; H, 4.43; N, 17.60 Found: C, 68.05; H, 4.37; N, 17.58

### EXAMPLE 2

To a stirred solution of 5-methoxy-3-(3-oxo-2,3-dihydropyridazin-6-yl)-2-phenylpyrazolo[1,5-a]pyridine (400 mg) in N,N-dimethylformamide (10 ml) was added 60%-sodium hydride (80.0 mg) at ambient temperature. After being stirred for 15 minutes, isopropyl iodide (290 g 1) was added to the mixture, and the mixture was stirred at ambient temperature for 18 hours. The mixture was partitioned between water and ethyl acetate. The organic layer was washed with water and brine, dried over magnesium sulfate, then evaporated in vacuo. The residue was purified by silica-gel column chromatography (n-hexane:ethyl acetate=2:1 to 1:1) to give 5-methoxy-3-(3-oxo-2-isopropyl-2,3-dihydropyridazin-6-yl)-2-phenylpyrazolo[1,5-a]pyridine (340 mg).

mp: 180.5-181.5° C. (AcOEt)

IR (nujol): 1648, 1570, 1538, 1523 cm⁻¹

NMR (DMSO-d6, δ): 1.34(6H,d,J=6.6 Hz), 3.88(3H,s), 5.10-5.35(1H,m), 6.76(1H,dd,J=2.8, 7.5 Hz), 6.83(1H,d,J=9.6 Hz), 7.06(1H,d,J=9.6 Hz), 7.28(1H,d,J=2.8 Hz), 7.40-7.65(5H,m), 8.69 (1H,d,J=7.5 Hz)

APCI/MS: 361[M+H]⁺

Anal.Calcd for C₂₁H₂₀N₄O₂: C, 69.98; H, 5.59; N, 15.55 Found: C, 70.48; H, 5.59; N, 15.64

### EXAMPLE 3

Boron tribromide (14.4 ml) was dissolved in dichloromethane (50 ml). To the solution was added dropwise a solution of 5-methoxy-3-(3-oxo-2-isopropyl-2,3-dihydropyridazin-6-yl)-2-phenylpyrazolo[1,5-a]pyridine (11.0 g) in dichloromethane (150 ml) over the period of 30 minutes. After being stirred at ambient temperature for 17 hours, the reaction mixture was poured into water (300 ml). The mixture was extracted with dichloromethane. The organic layer was washed with water and brine, and dried over magnesium sulfate, then evaporated in vacuo. The residue was triturated by ethyl acetate. The resultant crystals were collected by filtration to give 5-hydroxy-3-(3-oxo-2-isopropyl-2,3-dihydropyridazin-6-yl)-2-phenylpyrazolo[1,5-a]pyridine (6.59 g).

mp: >250° C. (AcOEt)

IR (nujol): 1646, 1560, 1540, 1521 cm⁻¹

NMR (DMSO-d6, δ): 1.34(6H,d,J=6.6 Hz), 5.10-5.34(1H,m), 6.66(1H,dd,J=2.6, 7.5 Hz), 6.79(1H,d,J=9.6 Hz), 6.99(1H,d,J=9.6 Hz), 7.17(1H,d,J=2.6 Hz), 7.34-7.65(5H,m), 8.62(1H,d,J=7.5 Hz), 10.66(1H,s)

APCI/MS: 347[M+H]⁺

Anal.Calcd for C₂₀H₁₈N₄O₂: C, 69.35; H, 5.24; N, 16.17 Found: C, 69.29; H, 5.24; N, 15.91

### EXAMPLE 4

To a solution of 5-hydroxy-3-(3-oxo-2-isopropyl-2,3-dihydropyridazin-6-yl)-2-phenylpyrazolo[1,5-a]pyridine (47.0 g) in N,N-dimethylformamide (470 ml) was added 60%-sodium hydride (16.3 g) at ambient temperature. After being stirred for 1 hour, a suspension of 2-(dimethylamino)ethylchloride hydrochloride (19.5 g) in N,N-dimethylformamide (200 ml) was added to the mixture, and the mixture was heated at 85° C. for 1.5 hours. The mixture was poured into water and extracted by ethyl acetate. The organic layer was washed with water and brine, and dried over magnesium sulfate, then evaporated in vacuo. The residue was purified by silica-gel column chromatography (ethyl acetate only, chloroform:methanol=9:1) to give 5-(2-dimethylamino)ethoxy-3-(3-oxo-2-isopropyl-2,3-dihydropyridazin-6-yl)-2-phenylpyrazolo[1,5-a]pyridine (37.6 g).

mp: 138-139° C. (AcOEt-IPE)

IR (nujol): 1648, 1585, 1538, 1280 cm⁻¹

NMR (DMSO-d6, δ): 1.33(6H,d,J=6.6 Hz), 2.23(6H,s), 2.69(2H,t,J=5.8 Hz), 4.15(2H,t,J=5.8 Hz), 5.10-5.36(1H,m), 6.75(1H,dd,J=2.7, 7.5 Hz), 6.84(1H,d,J=9.6 Hz), 7.08(1H,d,J=9.6 Hz), 7.28(1H,d,J=2.7 Hz), 7.36-7.64(5H,m), 8.67 (1H,d, J=7.5 Hz)

APCI/MS: 418[M+H]⁺

Anal.Calcd for C₂₄H₂₇N₅O₂: C, 69.04; H, 6.52; N, 16.77 Found: C, 69.35; H, 6.52; N, 16.82

### EXAMPLE 5

A mixture of 1-(5-methoxy-2-phenylpyrazolo[1,5-a]pyridin-3-yl)ethanone (1.20 g), glyoxylic acid monohydrate (1.66 g), and 1,4-dioxane (12 ml) was stirred at 85° C. for 24 hours. After being cool, to the reaction mixture was added hydrazine monohydrate (3.28 ml). Then the reaction mixture was stirred at 85° C. for 4 hours. To the mixture was added ice-water (48 ml), and was stirred for 2 hours at ambient temperature. The resultant precipitate was collected by filtration to give 5-methoxy-3-(3-oxo-2,3-dihydropyridazin-6-yl)-2-phenylpyrazolo[1,5-a]pyridine (493 mg).

IR (nujol): 1673, 1644, 1577, 1565, 1540, 1515 cm⁻¹

NMR (DMSO-d6, δ): 3.86(3H,s), 6.74(1H,dd,J=2.8, 7.6 Hz), 6.81(1H,d,J=9.9 Hz), 7.08(1H,d,J=9.8 Hz), 7.16(1H,d,J=2.6 Hz), 7.28-7.75(5H,m), 8.67(1H,d,J=7.6 Hz), 13.07(1H,s)

APCI/MS: 319[M+H]⁺

### EXAMPLE 6

The solution of 5-hydroxy-3-(3-oxo-2-isopropyl-2,3-dihydropyridazin-6-yl)-2-phenylpyrazolo[1,5-a]pyridine (80 mg), 4-(2-chloroethyl)morpholine hydrochloride (64.5 mg) and K₂CO₃ (192 mg) in N,N-dimethylformamide (5 ml) was stirred at 50° C. for 24 hours. The mixture was poured into water (30 ml) and the mixture was extracted with CHCl₃ (20 ml×3). The organic layer was washed with water and brine, and dried over magnesium sulfate, then evaporated in vacuo. The residue was purified by silica-gel (5 g) column chlomathography (CHCl₃: MeOH=9:1) to give 5-[2-(4-morpholinyl)ethoxy]-3-(3-oxo-2-isopropyl-2,3-dihydropyridazin-6-yl)-2-phenylpyrazolo[1,5-a]pyridine (48.0 mg).

mp: 160-162° C. (CHCl₃-n-Hexane)

NMR (DMSO-d6, δ): 1.32(6H,d,J=6.6 Hz), 2.40-2.50(4H,m), 2.76(2H,t,J=5.6 Hz), 3.58(4H,t,J=4.6 Hz), 4.19(2H,t,J=5.7 Hz), 5.10-5.35(1H,m), 6.76(1H,dd,J=2.7, 7.5 Hz), 6.84(1H,d,J=9.6 Hz), 7.10(1H,d,J=9.6 Hz), 7.28(1H,d,J=2.6 Hz), 7.40-7.70(5H,m), 8.67 (1H,d,J=7.5 Hz)

APCI/MS: 460[M+H]⁺

### EXAMPLE 7

To the solution of 5-hydroxy-3-(3-oxo-2-isopropyl-2,3-dihydropyridazin-6-yl)-2-phenylpyrazolo[1,5-a]pyridine (80 mg), and triethylamine (0.0644 ml) in tetrahydrofuran (3 ml) was added the solution of 4-methoxybenzenesulfonyl chloride (47.7 mg) in tetrahydrofuran (2 ml) at 0° C., and the mixture was stirred at <5° C. for 20 hours. To the mixture was added 1N—HCl (30 ml) and the mixture was extracted with AcOEt (20 ml×3). The organic layer was washed with sat.aq.NaHCO₃ and brine, and dried over magnesium sulfate, then evaporated in vacuo. The residue was purified by silica-gel (2.5 g) column chlomathography (CHCl₃ MeOH=19:1-9:1) to give 5-(4-methoxyphenylsulfonyl)oxy-3-(3-oxo-2-isopropyl-2,3-dihydropyridazin-6-yl)-2-phenylpyrazolo[1,5-a]pyridine.

mp: 155-157° C. (CHCl₃-n-Hexane)

NMR (DMSO-d6, δ): 1.26(6H,d,J=6.6 Hz), 3.86(3H,s), 5.10-5.35 (1H,m), 6.81(1H,dd,J=2.7, 7.5 Hz), 6.83(1H,d,J=9.7 Hz), 6.99(1H,d, J=9.6 Hz), 7.18(2H,td,J=2.6, 9.5 Hz), 7.40-7.65(6H,m), 7.86(2H, td, J=3.6, 9.5 Hz), 8.88(1H,d,J=7.5 Hz)

APCI/MS: 517 [M+H]⁺

### EXAMPLE 8

To the solution of 5-hydroxy-3-(3-oxo-2-isopropyl-2,3-dihydropyridazin-6-yl)-2-phenylpyrazolo[1,5-a]pyridine (150 mg) in N,N-dimethylformamide (1 ml) was added NaH (20.8 mg) at 0° C., and the mixture was stirred for 15 min at ambient temperature. To the mixture was added benzylbromide (0.0515 ml) at 0° C., and the mixture was stirred at 85° C. for 7 hours. To the mixture was added water (2 ml) and the mixture was extracted with CHCl₃ (5 ml). The organic layer was washed with water and passed through the Presep (diatomaceous earth, granular) column with CHCl₃, and evaporated. The residue was purified by silica-gel preparative TLC (AcOEt: n-Hexane=1:1) to give 5-benzyloxy-3-(3-oxo-2-isopropyl-2,3-dihydropyridazin-6-yl)-2-phenylpyrazolo[1,5-a]pyridine.

mp: 167.5-168.5° C. (AcOEt-n-Hexane)

NMR (DMSO-d6, δ): 1.28(6H,d,J=6.6 Hz), 5.05-5.40(3H,m), 6.75-6.90(2H,m), 7.07(1H,d,J=9.6 Hz), 7.25-7.70(11H,m), 8.71(1H,d, J=7.5 Hz)

APCI/MS: 487[M+H]⁺

### EXAMPLE 9

5-Phenethyloxy-3-(3-oxo-2-isopropyl-2,3-dihydropyridazin-6-yl)-2-phenylpyrazolo[1,5-a]pyridine was prepared by similar procedure as that of Example 8.

mp: 154.5-155.5° C. (AcOEt)

NMR (DMSO-d6, δ): 1.27(6H,d,J=6.6 Hz), 3.12(2H,t,J=6.8 Hz), 4.31(2H,t,J=6.9 Hz), 5.05-5.30(1H,m), 6.74(1H, d, J=2.6, 7.5 Hz), 6.83(1H,d,J=9.6 Hz), 7.08(1H,d,J=9.6 Hz), 7.10-7.40(6H,m), 7.40-7.65(5H,m), 8.67(1H,d,J=7.5 Hz)

APCI/MS: 451[M+H]⁺

### EXAMPLE 10

5-Isopropoxy-3-(3-oxo-2-isopropyl-2,3-dihydropyridazin-6-yl)-2-phenylpyrazolo[1,5-a]pyridine was prepared by similar procedure as that of Example 8.

mp: 162-163° C. (AcOEt-n-Hexane)

NMR (DMSO-d6, δ): 1.20-1.50(12H,m), 4.60-4.85(1H,m), 5.10-5.35(1H,m), 6.70(1H,dd,J=2.7, 7.5 Hz), 6.81(1H,d,J=9.6 Hz), 7.01(1H,d,J=9.7 Hz), 7.21(1H,d,J=2.6 Hz), 7.40-7.65(5H,m), 8.67 (1H,d, J=7.5 Hz)

APCI/MS: 389[M+H]⁺

### EXAMPLE 11

5-[3-{(2R)-2-Methoxymethyl-4-morpholinyl}propoxy]-3-(3-oxo-2-isopropyl-2,3-dihydropyridazin-6-yl)-2-phenylpyrazolo[1,5-a]pyridine (amorphous) was prepared by similar procedure as that of Example 8.

NMR (DMSO-d6, δ): 1.35(6H,d,J=6.6 Hz), 1.78(1H,t,J=10.6 Hz), 1.85-2.15(3H,m), 2.35-2.55(2H,m), 2.60-2.90(3H,m), 3.23(3H,s), 3.90-3.90(1H,m), 4.05-4.20(2H,m), 5.10-5.35(1H,m), 6.75(1H,dd, J=2.7, 7.5 Hz), 6.83(1H,d,J=9.6 Hz), 7.04(1H,d,J=9.6 Hz), 7.29(1H,d,J=2.5 Hz), 7.40-7.65(5H,m), 8.68(1H,d,J=7.5 Hz)

APCI/MS: 518[M+H]⁺

### EXAMPLE 12

5-(2-Pyridinylmethoxy)-3-(3-oxo-2-isopropyl-2,3-dihydropyridazin-6-yl)-2-phenylpyrazolo[1,5-a]pyridine was prepared by similar procedure as that of Example 8.

mp: 150-151° C. (AcOEt-n-Hexane)

NMR (DMSO-d6, δ): 1.25(6H,d,J=6.6 Hz), 5.05-5.30(1H,m), 5.31(2H,s), 6.82(1H,d,J=9.6 Hz), 6.89(1H,dd,J=2.8, 7.5 Hz), 7.03(1H,d,J=9.6 Hz), 7.25-7.65(8H,m), 7.80-7.95(1H,m), 8.60(1H,d,J=4.0 Hz), 8.73(1H,d,J=7.5 Hz)

APCI/MS: 438[M+H]⁺

### EXAMPLE 13

To the solution of 5-hydroxy-3-(3-oxo-2-isopropyl-2,3-dihydropyridazin-6-yl)-2-phenylpyrazolo[1,5-a]pyridine (250 mg) in N,N-dimethylformamide (1 ml) was added NaH (52 mg) at 0° C., and the mixture was stirred for 15 min at ambient temperature. To the mixture was added 2-(2-bromoethyl)-1H-isoindole-1,3(2H)-dione (238 mg) at 0° C., and the mixture was stirred at 70° C. for 24 hours. To the mixture was added water (5 ml) and the mixture was extracted with AcOEt (5 ml) twice. The organic layer was washed with water and brine, and dried over magnesium sulfate, then evaporated in vacuo. The residue was purified by silica-gel (30 g) column chlomathography (AcOEt:n-Hexane=1:1) to give 2-(2-{[3-(1-isopropyl-6-oxo-1,6-dihydro-3-pyridazinyl)-2-phenylpyrazolo[1,5-a]pyridin-5-yl]oxy}ethyl)-1H-isoindole-1,3(2H)-dione (150 mg).

NMR (DMSO-d6, δ): 1.30(6H,d,J=6.6 Hz), 4.06(2H,d,J=5.1 Hz), 4.36(2H, d, J=5.2 Hz), 5.10-5.35(1H,m), 6.66(1H, dd, J=2.6, 7.5 Hz), 6.84(1H,d,J=9.7 Hz), 7.10(1H,d,J=9.6 Hz), 7.23(1H,d,J=2.6 Hz), 7.30-8.00(9H,m), 8.64(1H,d,J=7.5 Hz)

APCI/MS: 520 [M+H]⁺

### EXAMPLE 14

To the solution of 2-(2-{[3-(1-isopropyl-6-oxo-1,6-dihydro-3-pyridazinyl)-2-phenylpyrazolo[1,5-a]pyridin-5-yl]oxy}ethyl)-1H-isoindole-1,3(2H)-dione (130 mg) in EtOH (3 ml) was added hydrazine monohydrate (0.0188 ml) at ambient temperature, and the mixture was stirred at ambient temperature for 3 hours. The mixture was filtered, and the mother solution was evaporated in vacuo. The residue was disolved in CH₂Cl₂ (5 ml) and the solution was washed with water and brine, and dried over magnesium sulfate, then evaporated in vacuo. The residue was purified by silica-gel (30 g) column chlomathography (CHCl₃:MeOH=9:1) to give 5-(2-aminoethoxy)-3-(3-oxo-2-isopropyl-2,3-dihydropyridazin-6-yl)-2-phenylpyrazolo[1,5-a]pyridine (45 mg).

NMR (DMSO-d6, δ): 1.34(6H,d,J=6.6 Hz), 2.94(2H,t,J=5.5 Hz), 4.01(2H,t,J=5.6 Hz), 5.10-5.40(1H,m), 6.76(1H,dd,J=2.8, 7.5 Hz), 6.83(1H,d,J=9.6 Hz), 7.06(1H,d,J=9.6 Hz), 7.26(1H,d,J=2.6 Hz), 7.30-7.65(5H,m), 8.68(1H, d, J=7.5 Hz)

APCI/MS: 390 [M+H]⁺

### EXAMPLE 15

5-(3-Dimethylaminopropoxy)-3-(3-oxo-2-isopropyl-2,3-dihydropyridazin-6-yl)-2-phenylpyrazolo[1,5-a]pyridine was prepared by similar procedure as that of Example 8.

mp: 125-126° C. (AcOEt-IPE)

NMR (DMSO-d6, δ): 1.34(6H,d,J=6.6 Hz), 1.80-2.05(2H,m), 2.16(6H,s), 2.38(2H,t,J=7.0 Hz), 4.11(2H,t,J=6.5 Hz), 5.05-5.40(1H,m), 6.75(1H,dd,J=2.7, 7.5 Hz), 6.83(1H,d,J=9.6 Hz), 7.06(1H,d,J=9.6 Hz), 7.29(1H,d,J=2.6 Hz), 7.40-7.65(5H,m), 8.67(1H,d,J=7.5 Hz)

APCI/MS: 432[M+H]⁺

### EXAMPLE 16

5-[2-(1-Piperidinyl)ethoxy]-3-(3-oxo-2-isopropyl-2,3-dihydropyridazin-6-yl)-2-phenylpyrazolo[1,5-a]pyridine was prepared by similar procedure as that of Example 8.

mp: 144-144.5° C. (AcOEt-IPE)

NMR (DMSO-d6, δ): 1.32(6H,d,J=6.6 Hz), 1.40-1.60(6H,m), 2.72(2H,t, J=6.1 Hz), 4.17(2H,t,J=5.7 Hz), 5.10-5.35(1H,m), 6.75(1H,dd, J=2.7, 7.5 Hz), 6.84(1H,d,J=9.6 Hz), 7.09(1H,d,J=9.6 Hz), 7.28(1H, d, J=2.2 Hz), 7.40-7.65(5H,m), 8.67(1H,d,J=7.5 Hz)

APCI/MS: 458[M+H]⁺

### EXAMPLE 17

5-[2-(1-Pyrrolidinyl)ethoxy]-3-(3-oxo-2-isopropyl-2,3-dihydropyridazin-6-yl)-2-phenylpyrazolo[1,5-a]pyridine was prepared by similar procedure as that of Example 8.

mp: 149-150° C. (AcOEt —IPE)

NMR (DMSO-d6,δ): 1.33(6H,d,J=6.6 Hz), 1.69(4H,qn,J=3.4 Hz), 2.85(2H,t,J=5.8 Hz), 4.17(2H,t,J=5.9 Hz), 5.05-5.35(1H,m), 6.76(1H,dd,J=2.7, 7.6 Hz), 6.83(1H,d,J=9.6 Hz), 7.08(1H,d, J=9.6 Hz), 7.28(1H,d,J=2.6 Hz), 7.40-7.65(5H,m), 8.67(1H,d, J=7.6 Hz)

APCI/MS: 444[M+H]⁺

### EXAMPLE 18

5-[2-{(3R,5S)-3,5-Dimethyl-4-morpholinyl}ethoxy]-3-(3-oxo-2-isopropyl-2,3-dihydropyridazin-6-yl)-2-phenylpyrazolo[1,5-a]pyridine was prepared by similar procedure as that of Example 8.

mp: 145-146° C. (AcOEt-IPE)

NMR (DMSO-d6,δ): 0.99(6H,d,J=6.2), 1.34(6H,d,J=6.6 Hz), 2.50-2.75(2H,m), 2.90-2.75(4H,m), 3.62(2H,dd,J=2.9, 11.1 Hz), 4.10(2H, t,J=6.0 Hz), 5.10-5.35(1H, m), 6.73(1H,dd,J=2.7, 7.5 Hz), 6.84(1H, d,J=9.6 Hz), 7.05(1H,d,J=9.6 Hz), 7.31(1H,d,J=2.6 Hz), 7.35-7.65(5H,m), 8.68(1H,d,J=7.5 Hz)

APCI/MS: 488[M+H]⁺

### EXAMPLE 19

5-(4-Pyridinylmethoxy)-3-(3-oxo-2-isopropyl-2,3-dihydropyridazin-6-yl)-2-phenylpyrazolo[1,5-a]pyridine was prepared by similar procedure as that of Example 8.

mp: 218-219.5° C. (AcOEt-n-Hexane)

NMR (DMSO-d6, δ): 1.22(6H,d,J=6.6 Hz), 5.05-5.30(1H,m), 5.34(2H,s), 6.83(1H,d,J=9.6 Hz), 6.90(1H,dd,J=2.8, 7.5 Hz), 7.05(1H,d,J=9.6 Hz), 7.27(1H,d,J=2.5 Hz), 7.40-7.65(7H,m), 8.61(2H,dd,J=1.6, 4.4 Hz), 8.75(1H,d,J=70.5 Hz)

APCI/MS: 438[M+H]⁺

### EXAMPLE 20

5-(3-Pyridinylmethoxy)-3-(3-oxo-2-isopropyl-2,3-dihydropyridazin-6-yl)-2-phenylpyrazolo[1,5-a]pyridine was prepared by similar procedure as that of Example 8.

mp: 145-146° C. (AcOEt-n-Hexane)

NMR (DMSO-d6, δ): 1.28(6H,d,J=6.6 Hz), 5.10-5.30(1H,m), 5.28(2H,s), 6.80-6.95(2H,m), 7.10(1H,d,J=9.6 Hz), 7.36(1H,d, J=2.6 Hz), 7.40-7.70(6H,m), 7.85-8.00(1H,dd,J=1.6, 4.8 Hz), 8.59(2H,m), 8.70-8.80(2H,m)

APCI/MS: 438[M+H]⁺

### EXAMPLE 21

5-Propoxy-3-(3-oxo-2-isopropyl-2,3-dihydropyridazin-6-yl)-2-phenylpyrazolo[1,5-a]pyridine was prepared by similar procedure as that of Example 8.

mp: 161.5-162.5° C. (AcOEt)

NMR (DMSO-d6, δ): 1.00(3H,t,J=7.4 Hz), 1.34(6H,d,J=6.6 Hz), 1.80(2H,q,J=7.2 Hz), 4.03(2H,t,J=6.6 Hz), 5.10-5.35(1H m), 6.70-6.80(1H,m), 6.83(1H,d,J=9.6 Hz), 7.06(1H,d,J=9.7 Hz), 7.25-7.35(1H,m), 7.45-7.70(5H,m), 8.68(1H,d,J=7.5 Hz)

APCI/MS: 389[M+H]⁺

### EXAMPLE 22

5-Isopentyloxy-3-(3-oxo-2-isopropyl-2,3 -dihydropyridazin-6-yl)-2-phenylpyrazolo[1,5-a]pyridine was prepared by similar procedure as that of Example 8.

mp: 163-163.5° C. (AcOEt)

NMR (DMSO-d6, δ): 0.95(6H,d,J=6.3 Hz), 1.34(6H,d,J=6.6 Hz), 1.60-1.90(3H,m), 4.09(2H,t,J=6.6 Hz), 5.10-5.35(1H,m), 6.74(1H, dd,J=2.7, 7.5 Hz), 6.83(1H,d,J=9.6 Hz), 7.05(1H,d,J=9.6 Hz), 7.40-7.65(5H,m), 8.67(1H,d,J=7.5 Hz)

APCI/MS: 417[M+H]⁺

### EXAMPLE 23

5-(2-Cyclohexylethoxy)-3-(3-oxo-2-isopropyl-2,3-dihydropyridazin-6-yl)-2-phenylpyrazolo[1,5-a]pyridine was prepared by similar procedure as that of Example 8.

mp: 146-147° C. (AcOEt)

NMR (DMSO-d6, δ): 0.80-1.10(2H,m), 1.10-1.30(3H,m), 1.33(6H,d, J=6.6 Hz), 1.40-1.60(1H,m), 1.60-1.85(6H,m), 4.10(2H,t, J=6.7 Hz), 5.10-5.40(1H,m), 6.74(1H,dd,J=2.7, 7.6 Hz), 6.83(1H,d,J=9.7 Hz), 7.06(1H,d,J=9.7 Hz), 7.29(1H,d,J=2.4 Hz), 7.40-7.65(5H,m), 8.67(1H,d,J=7.6 Hz)

APCI/MS: 457[M+H]⁺

### EXAMPLE 24

5-[2-{(2R,6S)-2,6-Dimethyl-4-morpholinyl}ethoxy]-3-(3-oxo-2-isopropyl-2,3-dihydropyridazin-6-yl)-2-phenylpyrazolo[1,5-a]pyridine was prepared by similar procedure as that of Example 8.

mp: 157.5-158° C. (AcOEt)

NMR(DMSO-d6, δ): 1.04(6H,d,J=6.3 Hz), 1.32(6H,d,J=6.6 Hz), 1.74(2H,t,J=10.6 Hz), 2.60-2.95(4H,m), 3.40-3.75(2H,m), 4.19(2H,t,J=5.6 Hz), 5.05-5.35(1H,m), 6.76(1H,dd,J=2.7, 7.5 Hz), 6.84(1H,d,J=9.7 Hz), 7.10(1H,d,J=9.7 Hz), 7.27(1H,d,J=2.6 Hz), 7.40-7.65(5H,m), 8.67(1H,d,J=7.5 Hz)

APCI/MS: 488[M+H]⁺

### EXAMPLE 25

5-[2-(Diethylamino)ethoxy]-3-(3-oxo-2-isopropyl-2,3-dihydropyridazin-6-yl)-2-phenylpyrazolo[1,5-a]pyridine was prepared by similar procedure as that of Example 8.

mp: 92.5-94.5° C. (Et₂O-n-Hexane)

NMR(DMSO-d6, δ): 0.98(6H,t, J=7.1 Hz), 1.33(6H,d,J=6.6 Hz), 2.52(4H,q,J=6.9 Hz), 2.84(2H,t,J=6.1 Hz), 4.11(2H,t,J=6.1 Hz), 5.10-5.40(1H,m), 6.74(1H,dd,J=2.7, 7.5 Hz), 6.83(1H,d,J=9.6 Hz), 7.07(1H,d,J=9.6 Hz), 7.29(1H,d,J=2.6 Hz), 7.40-7.65(5H,m), 8.67(1H,d,J=7.5 Hz)

APCI/MS: 446[M+H]⁺

### EXAMPLE 26

5-Ethoxy-3-(3-oxo-2-isopropyl-2,3-dihydropyridazin-6-yl)-2-phenylpyrazolo[1,5-a]pyridine was prepared by similar procedure as that of Example 8.

mp: 181-182° C. (AcOEt)

NMR(DMSO-d6, δ): 1.33(6H,d,J=6.6 Hz), 1.40(3H,t,J=6.9 Hz), 4.14(2H,t,J=7.0 Hz), 5.10-5.35(1H,m), 6.74(1H,dd,J=2.8, 7.5 Hz), 6.82(1H,d,J=9.6 Hz), 7.05(1H,d,J=9.7 Hz), 7.26(1H,d,J=2.6 Hz), 7.40-7.65(5H,m), 8.67(1H,d,J=7.5 Hz)

APCI/MS: 375[M+H]⁺

### EXAMPLE 27

5-(2-Oxopropoxy)-3-(3-oxo-2-isopropyl-2,3-dihydropyridazin-6-yl)-2-phenylpyrazolo[1,5-a]pyridine was prepared by similar procedure as that of Example 8.

mp: 172.5-173.5° C. (AcOEt-n-Hexane)

NMR(DMSO-d6, δ): 1.31(6H,d,J=6.6 Hz), 2.17(3H,s), 4.98(2H,s), 5.10-5.35(1H,m), 6.75-6.90(2H,m), 7.00-7.20(2H,m), 7.40-7.65(5H,m), 8.71(1H,d,J=7.6 Hz)

APCI/MS: 403[M+H]⁺

### EXAMPLE 28

5-[2-(2-Oxo-1-pyrrolidinyl)ethoxy]-3-(3-oxo-2-isopropyl-2,3-dihydropyridazin-6-yl)-2-phenylpyrazolo [1,5-a]pyridine was prepared by similar procedure as that of Example 8.

mp: 162-163° C. (AcOEt-n-Hexane)

NMR(DMSO-d6, δ): 1.48(6H,d,J=6.6 Hz), 2.07(2H,qn,J=7.6 Hz), 2.42(2H,t,J=8.0 Hz), 3.59(2H,t,J=7.0 Hz), 3.76(2H,t,J=5.1 Hz), 4.18(2H,t,J=5.1 Hz), 5.30-5.60(1H,m), 6.59(1H,dd, J=2.7, 7.5 Hz), 6.73(1H,d,J=9.6 Hz), 6.96(1H,d,J=9.6 Hz), 7.33(1H,d,J=2.7 Hz), 7.40-7.55(3H,m), 7.55-7.70(2H,m), 8.36(1H,d,J=7.5 Hz)

APCI/MS: 458[M+H]⁺

### EXAMPLE 29

tert-Butyl 4-{[3-(3-oxo-2-isopropyl-2,3-dihydropyridazin-6-yl)-2-phenylpyrazolo[1,5-a]pyridin-5-yl]oxy}-1-piperidinecarboxylate was prepared by similar procedure as that of Example 8.

mp: 176-179° C. (AcOEt)

NMR(DMSO-d6, δ): 1.34(6H,d,J=6.6 Hz), 1.41(9H,s), 1.50-1.80 (2H,m), 1.90-2.15(2H,m), 3.05-3.25(2H,m), 3.60-3.85(2H,m), 4.55-4.80(1H,m), 5.10-5.35(1H,m), 6.75(1H,dd,J=2.7, 7.5 Hz), 6.82(1H,d,J=9.6 Hz), 7.03(1H,d,J=9.6 Hz), 7.29(1H,d,J=2.6 Hz), 7.40-7.55(5H,m), 8.69(1H,d,J=7.5 Hz)

ESI/MS: 552[M+Na]⁺

### EXAMPLE 30

To the solution of tert-Butyl 4-{[3-(3-oxo-2-isopropyl-2,3-dihydropyridazin-6-yl)-2-phenylpyrazolo[1,5-a]pyridin-5-yl]oxy}-1-piperidinecarboxylate (1.32 g) in AcOEt (26 ml) was added 4N—HCl/AcOEt (6.23 ml), and the mixture was stirred at 65° C. for 2.5 hours. After being cool, the resultant precipitate was collected by filtration, and washed with AcOEt, then dried in vacuo at ambient temperature for 15 hours to give 5-(4-Piperidinyloxy)-3-(3-oxo-2-isopropyl-2,3-dihydropyridazin-6-yl)-2-phenylpyrazolo [1,5-a]pyridine hydrochloride (1.06 g).

NMR(DMSO-d6, δ): 1.32(6H,d,J=6.6 Hz), 1.80-2.05(2H,m), 2.05-2.30(2H,m), 2.90-3.20(2H,m), 3.20-3.40(2H,m), 4.65-4.90(1H,m), 5.10-5.35(1H,m), 6.70-6.90(2H,m), 7.08(1H,d,J=9.6 Hz), 7.25(1H, d,J=2.6 Hz), 7.40-7.65(5H,m), 8.73(1H,d,J=7.5 Hz), 8.91 (2H,br,s)

APCI/MS: 430[M−HCl+H]⁺

### EXAMPLE 31

The solution of 5-hydroxy-3-(3-oxo-2-isopropyl-2,3-dihydropyridazin-6-yl)-2-phenylpyrazolo[1,5-a]pyridine (300 mg), 2-bromopyridine (0.124 ml), K₂CO₃ (359 mg) in DMF (4 ml) was stirred at 120° C. for 18 hours. After being cooled, to the mixture was added water (20 ml) and the mixture was extracted with AcOEt (15 ml×3). The organic layer was washed with water and brine, and dried over magnesium sulfate, then evaporated in vacuo. The residue was purified by silica-gel (15 g) column chlomathography (AcOEt:n-Hexane=1:1) to give 5-(2-pyridinyloxy)-3-(3-oxo-2-isopropyl-2,3-dihydropyridazin-6-yl)-2-phenylpyrazolo[1,5-a]pyridine (113.1 mg).

mp: 140.5-141.5° C. (AcOEt)

NMR(DMSO-d6, δ): 1.13(6H,d,J=6.6 Hz), 5.00-5.25(1H,m), 6.79(1H, d,J=9.7 Hz), 6.90-7.10(2H,m), 7.20-7.35(2H,m), 7.45-7.70(6H,m), 7.90-8.10(1H,m), 8.30-8.40(1H,m), 8.87(1H,d, J=7.5 Hz)

APCI/MS: 424[M+H]⁺

### EXAMPLE 32

Benzyl {[3-(3-oxo-2-isopropyl-2,3-dihydropyridazin-6-yl)-2-phenylpyrazolo[1,5-a]pyridin-5-yl]oxy}acetate was prepared by similar procedure as that of Example 8.

mp: 174-174.5° C. (AcOEt)

NMR(DMSO-d6, δ): 1.26(6H,d,J=6.6 Hz), 5.03(2H,s), 5.15-5.30 (3H, m), 6.75-6.90(2H,m), 7.05(1H,d,J=9.6 Hz), 7.17(1H,d,J=2.6 Hz), 7.25-7.40(5H,m), 7.40-7.65(5H,m), 8.72(1H,d,J=7.6 Hz)

APCI/MS: 495[M+H]⁺

### EXAMPLE 33

To the solution of 5-hydroxy-3-(3-oxo-2-isopropyl-2,3-dihydropyridazin-6-yl)-2-phenylpyrazolo[1,5-a]pyridine (200 mg), 2-cyclopentylethanol (0.086 ml), PPh₃ (303 mg) in THF (4 ml) was added dropwise DEAD (0.182 ml) at 0° C. The mixture was stirred at ambient temperature for 36 hours. To the mixture was added saturated sodium hydrogen carbonate solution and the mixture was extracted with AcOEt. The organic layer was washed with brine, and dried over magnesium sulfate, then evaporated in vacuo. The residue was purified by silica-gel (40 g) column chlomathography (CHCl₃:MeOH=50:1) to give 5-(2-cyclopentylethoxy)-3-(3-oxo-2-isopropyl-2,3-dihydropyridazin-6-yl)-2-phenylpyrazolo[1,5-a]pyridine (14.1 mg).

mp: 159.5-160.5° C. (Et₂O-n-Hexane)

NMR(DMSO-d6, δ): 1.05-1.30(2H,m), 1.34(6H,d,J=6.6 Hz), 1.45-1.70 (4H,m), 1.70-2.05(5H,m), 4.08(2H,t,J=6.6 Hz), 5.10-5.35(1H,m), 6.74(1H,dd,J=2.7, 7.5 Hz), 6.82(1H,d,J=9.6 Hz), 7.04(1H,d,J=9.6 Hz), 7.30(1H,d,J=2.6 Hz), 7.45-7.65(5H,m), 8.67(1H,d,J=7.5 Hz)

APCI/MS: 443[M+H]⁺

### EXAMPLE 34

5-[2-(1-Aziridinyl)ethoxy]-3-(3-oxo-2-isopropyl-2,3-dihydropyridazin-6-yl)-2-phenylpyrazolo[1,5-a]pyridine was prepared by similar procedure as that of Example 33.

mp: 159-160° C. (AcOEt-n-Hexane)

NMR(DMSO-d6, δ): 1.25-1.45(6H,m), 1.85-2.30(4H, m), 3.85-4.10(2H, m), 5.10-5.30(1H,m), 5.90-6.05(1H,m), 6.73(1H,dd,J=2.7, 7.5 Hz), 6.82(1H,d,J=9.6 Hz), 7.02(1H,d,J=9.6 Hz), 7.35-7.70(5H,m), 8.69(1H,d,J=7.5 Hz)

APCI/MS: 416[M+H]⁺

### EXAMPLE 35

5-(4-Pyridinyloxy)-3-(3-oxo-2-isopropyl-2,3-dihydropyridazin-6-yl)-2-phenylpyrazolo[1,5-a]pyridine was prepared by similar procedure as that of Example 31.

mp: 218.5-219.5° C. (AcOEt-n-Hexane)

NMR(DMSO-d6, δ): 1.07(6H,d,J=6.6 Hz), 5.00-5.23(1H,m), 6.79(1H, d,J=9.7 Hz), 7.01(1H,d,J=9.7 Hz), 7.05(1H,dd,J=2.5, 7.5 Hz), 7.29(2H,dd,J=1.6, 4.6 Hz), 7.42(1H,d,J=2.2 Hz), 7.45-7.65(5H,m), 8.61(2H,dd,J=1.6, 4.6 Hz), 8.91(1H,d,J=7.5 Hz)

APCI/MS: 424[M+H]⁺

### EXAMPLE 36

The solution of 5-hydroxy-3-(3-oxo-2-isopropyl-2,3-dihydropyridazin-6-yl)-2-phenylpyrazolo[1,5-a]pyridine (346 mg), 3-(acetylamino)phenylboronic acid (180 mg), Cu(OAc)₂ (181 mg), Et₃N (0.696 ml), 4AMS (125 mg) in CH₂Cl₂ (5 ml) was refluxed for 20 hours. After being cooled, the mixture was filtrated by Celite, and washed with AcOEt. The solvent was evaporated in vacuo. The residue was purified by silica-gel (35 g) column chlomathography (AcOEt:n-Hexane=1:1→3:2→2:1→3:1→1:0) to give N-(3-{[3-(3-oxo-2-isopropyl-2,3-dihydropyridazin-6-yl)-2-phenylpyrazolo[1,5-a]pyridin-5-yl]oxy}phenyl)acetamide (41.7 mg).

mp: 177.5-178.5° C. (AcOEt)

NMR(DMSO-d6, δ): 0.99(6H,d,J=6.6 Hz), 2.04(3H,s), 4.97-5.15(1H, m), 6.72(1H,d,J=9.7 Hz), 6.88(1H,d,J=9.7 Hz), 6.90-7.00(1H,m), 7.02(1H,dd,J=2.6, 7.5 Hz), 7.16(1H,d,J=7.5 Hz), 7.41(2H,d, J=5.1 Hz), 7.45-7.65(5H,m), 7.65(1H,s), 8.84(1H,d,J=7.5 Hz), 10.13(1H,s)

APCI/MS: 480[M+H]⁺

### EXAMPLE 37

5-Phenoxy-3-(3-oxo-2-isopropyl-2,3-dihydropyridazin-6-yl)-2-phenylpyrazolo[1,5-a]pyridine was prepared by similar procedure as that of Example 36.

mp: 181-182° C. (AcOEt-n-Hexane)

NMR(DMSO-d6, δ): 0.97(6H,d,J=6.6 Hz), 4.90-5.20(1H,m), 6.72(1H, d,J=9.7 Hz), 6.88(1H,d,J=9.7 Hz), 6.95-7.05(1H,m), 7.12(1H,d, J=2.2 Hz), 7.25-7.40(3H,m), 7.45-7.65(7H,m), 8.83(1H,d,J=7.0 Hz)

APCI/MS: 423[M+H]⁺

### EXAMPLE 38

Ethyl {[3-(3-oxo-2-isopropyl-2,3-dihydropyridazin-6-yl)-2-phenylpyrazolo[1,5-a]pyridin-5-yl]oxy}acetate was prepared by similar procedure as that of Example 8.

mp: 193-194° C. (AcOEt)

NMR(DMSO-d6, δ): 1.25(3H,t,J=7.1 Hz), 1.31(6H,d,J=6.6 Hz), 4.18(2H,q,J=7.1 Hz), 4.95(2H,s), 5.10-5.35(1H,m), 6.75-6.90(2H, m), 7.03(1H,d,J=9.6 Hz), 7.18(1H,d,J=2.7 Hz), 7.40-7.65(5H,m), 8.73(1H,d,J=7.6 Hz)

APCI/MS: 433[M+H]⁺

### EXAMPLE 39

To the solution of ethyl [{3-(3-oxo-2-isopropyl-2,3-dihydropyridazin-6-yl)-2-phenylpyrazolo[1,5-a]pyridin-5-yl}oxy]acetate (2.30 g) in EtOH (20 ml) was added 1N-aq.NaOH (6 ml), and the mixture was stirred at 70° C. for 30 minutes. The reaction mixture was acidified with 1N-aq.HCl to pH 1-2, and extracted with AcOEt twice, and the organic layer was washed with water twice and brine, and dried over magnesium sulfate, then evaporated in vacuo to give [{3-(3-oxo-2-isopropyl-2,3-dihydropyridazin-6-yl)-2-phenylpyrazolo[1,5-a]pyridin-5-yl}oxy]acetic acid (2.13 g).

mp: 219.5-218.3° C. (75% aq.EtOH)

NMR(DMSO-d6, δ): 1.33(6H,d,J=6.6 Hz), 4.83(2H,s), 5.10-5.35(1H, m), 6.70-6.90(2H,m), 7.01(1H,d,J=9.6 Hz), 7.19(1H,d,J=2.6 Hz), 7.40-7.70(5H,m), 8.71(1H,d,J=7.5 Hz), 13.25(1H,br,s)

APCI/MS: 405[M+H]⁺

### EXAMPLE 40

To the solution of [{3-(3-oxo-2-isopropyl-2,3-dihydropyridazin-6-yl)-2-phenylpyrazolo[1,5-a]pyridin-5-yl}oxy]acetic acid (500 mg), 2-(1H-benzotriazol-1-yl)-1,1,3,3-tetramethyluronium tetrafluoroborate; TBTU (794 mg) in DMF (2.5 ml) was added ethyldiisopropylamine (0.646 ml), and the mixture was stirred at ambient temperature for 15 minutes. To the mixture was added N,N-dimethylamine hydrochloride (151 mg), and the mixture was stirred at ambient temperature for 1.5 hours. To the reaction mixture was added saturated sodium hydrogen carbonate solution, and was acidified with 1N—HCl, then was extracted with AcOEt. The organic layer was washed with water twice and saturated sodium hydrogen carbonate solution, water and brine, then dried over magnesium sulfate, and evaporated in vacuo. The residue was purified by silica-gel (11 g) column chlomathography (CHCl₃:MeOH=40:1-20:1) to give 2-{[3-(3-oxo-2-isopropyl-2,3-dihydropyridazin-6-yl)-2-phenylpyrazolo[1,5-a]pyridin-5-yl]oxy}-N,N-dimethylacetamide (390 mg).

mp: 189-190° C. (AcOEt-n-Hexane)

NMR(DMSO-d6, δ): 1.33(6H,d,J=6.6 Hz), 2.96(3H,s), 2.98(3H,s), 5.01(2H,s), 5.10-5.30(1H,m), 6.75-6.85(2H,m), 6.97(1H,d, J=9.6 Hz), 7.15(1H,d,J=2.5 Hz), 7.40-7.70(5H,m), 8.69(1H,d, J=7.5 Hz)

APCI/MS: 432[M+H]⁺

### EXAMPLE 41

N,N-Diethyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 40.

mp: 125-126.5° C. (AcOEt-n-Hexane)

NMR(DMSO-d6, δ): 1.03(3H,t,J=7.1 Hz), 1.17(3H,dt,J=2.7, 7.1 Hz), 1.33(6H,d,J=6.6 Hz), 3.20-3.45(4H,m), 4.97(2H,s), 5.05-5.35(1H, m), 6.70-6.90(2H,m), 7.02(1H,d,J=9.6 Hz), 7.17(1H,d,J=2.5 Hz), 7.40-7.65(5H,m), 8.70(1H,d,J=7.5 Hz)

APCI/MS: 460[M+H]⁺

### EXAMPLE 42

tert-Butyl 4-({[3-(3-oxo-2-isopropyl-2,3-dihydropyridazin-6-yl)-2-phenylpyrazolo[1,5-a]pyridin-5-yl]oxy}acetyl)-1-piperazinecarboxylate was prepared by similar procedure as that of Example 40.

mp: 139.5-143.5° C. (AcOEt-n-Hexane)

NMR(DMSO-d6, δ): 1.32(6H,d,J=6.6 Hz), 1.42(9H,s), 2.69(4H,s), 3.25-3.55(4H,m), 5.02(2H,s), 5.10-5.35(1H,m), 6.70-6.90(2H,m), 7.03(1H,d,J=9.6 Hz), 7.17(1H,d,J=2.5 Hz), 7.35-7.65(5H,m), 8.70(1H,d,J=7.5 Hz)

ESI/MS: 495[M+Na]⁺

### EXAMPLE 43

5-[2-Oxo-2-(1-piperazinyl)]ethoxy-3-(3-oxo-2-isopropyl-2,3-dihydropyridazin-6-yl)-2-phenylpyrazolo[1,5-a]pyridine hydrochloride was prepared by similar procedure as that of Example 30.

mp: 191-193.5° C. (IPA)

NMR(DMSO-d6, δ): 1.32(6H,d,J=6.6 Hz), 3.00-3.25(4H,m), 3.55-3.85(4H,m), 5.08(2H,s), 5.10-5.35(1H,m), 6.75-6.90(2H,m), 7.04(1H,d,J=9.6 Hz), 7.16(1H,d,J=2.5 Hz), 7.40-7.65(5H,m), 8.71(1H,d,J=7.5 Hz), 9.21(2H,br,s)

APCI/MS: 473[M−HCl+H]⁺

### EXAMPLE 44

2-{[3-(3-Oxo-2-isopropyl-2,3-dihydropyridazin-6-yl)-2-phenylpyrazolo[1,5-a]pyridin-5-yl]oxy}-N-phenylacetamide was prepared by similar procedure as that of Example 40.

mp: 208-209° C. (AcOEt)

NMR(DMSO-d6, δ): 1.23(6H,d, J=6.6 Hz), 4.86(2H,s), 5.05-5.30(1H, m), 6.79(1H,d,J=9.6 Hz), 6.85-6.95(1H,m), 6.95-7.15(2H,m), 7.23(1H,d,J=2.6 Hz), 7.33(2H,t,J=7.8 Hz), 7.40-7.75(7H,m), 8.75(1H,d,J=7.6 Hz), 10.18(1H,s)

APCI/MS: 480[M+H]⁺

### EXAMPLE 45

N-Isobutyl-2-{[3-(3-oxo-2-isopropyl-2,3-dihydropyridazin-6-yl)-2-phenylpyrazolo [1,5-a]pyridin-5-yl]oxy}acetamide was prepared by similar procedure as that of Example 40.

mp: 167.5-168.5° C. (AcOEt)

NMR(DMSO-d6, δ): 0.79(6H,d,J=6.7 Hz), 1.30(6H,d,J=6.6 Hz), 1.55-1.85(1H,m), 2.95(2H,t,J=6.4 Hz), 4.62(2H,s), 5.05-5.35(1H, m), 6.75-6.95(2H,m), 7.08(1H,d,J=9.6 Hz), 7.16(1H,d,J=2.6 Hz), 7.35-7.65(5H,m), 8.16(1H,t,J=5.7 Hz), 8.73(1H,d,J=7.5 Hz)

APCI/MS: 460[M+H]⁺

### EXAMPLE 46

5-(Tetrahydropyran-4-yl)oxy-3-(3-oxo-2-isopropyl-2,3-dihydropyridazin-6-yl)-2-phenylpyrazolo[1,5-a]pyridine was prepared by similar procedure as that of Example 8.

mp: 210.5-211° C. (AcOEt)

NMR(DMSO-d6, δ): 1.35(6H,d,J=6.6 Hz), 1.55-1.80(2H,m), 1.95-2.20(2H,m), 3.80-4.00(2H,m), 4.55-4.80(1H,m), 5.10-5.35(1H,m), 6.75(1H,dd,J=2.7, 7.6 Hz), 6.81(1H,d,J=9.7 Hz), 7.31(1H,d, J=2.6 Hz), 7.40-7.65(5H,m), 8.69(1H,d,J=7.5 Hz)

ESI/MS: 453[M+Na]⁺

### EXAMPLE 47

5-[(5-Methoxy-2-pyridinyl)oxy]-3-(3-oxo-2-isopropyl-2,3-dihydropyridazin-6-yl)-2-phenylpyrazolo[1,5-a]pyridine was prepared by similar procedure as that of Example 8.

mp: 148-150° C. (AcOEt)

NMR(DMSO-d6, δ): 1.09(6H,d,J=6.6 Hz), 3.86(3H,s), 4.95-5.25(1H, m), 6.76(1H,d,J=9.6 Hz), 6.90-7.10(2H,m), 7.25-7.40(2H,m), 7.40-7.75(6H,m), 7.75-7.90(1H,m), 8.84(1H,d,J=7.5 Hz)

ESI/MS: 476[M+Na]⁺

### EXAMPLE 48

5-[(5-Nitro-2-pyridinyl)oxy]-3-(3-oxo-2-isopropyl-2,3-dihydropyridazin-6-yl)-2-phenylpyrazolo[1,5-a]pyridine was prepared by similar procedure as that of Example 8.

mp: 182-183.5° C. (AcOEt)

NMR(DMSO-d6, δ): 1.20(6H,d,J=6.6 Hz), 5.00-5.30(1H,m), 6.84(1H, d,J=9.6 Hz), 7.00-7.20(2H,m), 7.35-7.65(6H,m), 7.72(1H,d, J=2.4 Hz), 8.71(1H,dd,J=2.9, 9.1 Hz), 8.94(1H,d,J=7.5 Hz), 9.10(1H, d,J=2.8 Hz)

ESI/MS: 491[M+Na]⁺

### EXAMPLE 49

Methyl {([3-(3-oxo-2-isopropyl-2,3-dihydropyridazin-6-yl)-2-phenylpyrazolo[1,5-a]pyridin-5-yl]oxy}acetate was prepared by similar procedure as that of Example 19.

NMR(DMSO-d6, δ): 1.32(6H,d,J=6.6 Hz), 3.73(3H,s), 4.97(2H,s), 5.10-5.35(1H,m), 6.750-6.90(2H,m), 7.02(1H,d,J=9.6 Hz), 7.19(1H, d,J=2.7 Hz), 7.40-7.65(5H,m), 8.72(1H,d,J=7.5 Hz)

ESI/MS: 441[M+Na]⁺

### EXAMPLE 50

To the mixture of [{3-(3-oxo-2-isopropyl-2,3-dihydropyridazin-6-yl)-2-phenylpyrazolo[1,5-a]pyridin-5-yl}oxy]acetic acid (200 mg), WSC—HCl (114 mg), HOBt (80.2 mg) in DMF (1.0 ml) was added 1-methylpiperadine (0.0603 ml) at 0° C., and the mixture was stirred at ambient temperature for 3.5 hours. To the reaction mixture was added water, and the mixture was extracted with AcOEt three times, then the organic layer was washed with water twice and brine, and dried over magnesium sulfate, and evaporated in vacuo. The residue was purified by silica-gel (8 g) column chlomathography (CHCl₃:MeOH=9:1) to give 5-[2-(4-methyl-1-piperazinyl)-2-oxo]ethoxy-3-(3-oxo-2-isopropyl-2,3-dihydropyridazin-6-yl)-2-phenylpyrazolo[1,5-a]pyridine (151.0 mg).

mp: 147-148° C. (AcOEt-n-Hexane)

NMR(DMSO-d6, δ): 1.32(6H,d,J=6.6 Hz), 2.19(3H,s), 2.10-2.45(4H, m), 3.25-3.60(4H,m), 5.00(2H,s), 5.05-5.35(1H,m), 6.70-6.90(2H, m), 7.03(1H,d,J=9.6 Hz), 7.16(1H,d,J=2.6 Hz), 7.35-7.65(5H,m), 8.69 (1H,d, J=7.5 Hz)

ESI/MS: 487[M+H]⁺

### EXAMPLE 51

5-[2-(4-Morpholinyl)-2-oxo]ethoxy-3-(3-oxo-2-isopropyl-2,3-dihydropyridazin-6-yl)-2-phenylpyrazolo[1,5-a]pyridine was prepared by similar procedure as that of Example 50.

mp: 192.5-193.5° C. (AcOEt)

NMR(DMSO-d6, δ): 1.33(6H,d,J=6.6 Hz), 3.35-3.75(8H,m), 5.02(2H, s), 5.10-5.35(1H,m), 6.70-6.90(2H,m), 7.03(1H,d,J=9.6 Hz), 7.17(1H,d,J=2.6 Hz), 7.35-7.65(5H,m), 8.70(1H,d,J=7.5 Hz)

ESI/MS: 474[M+Na]⁺

### EXAMPLE 52

N,N-Bis(2-ethoxyethyl)-2-[{3-(3-oxo-2-isopropyl-2,3-dihydropyridazin-6-yl)-2-phenylpyrazolo[1,5-a]pyridin-5-yl}oxy]acetamide (amorphous) was prepared by similar procedure as that of Example 50.

NMR(DMSO-d6, δ): 0.90-1.15(6H,m), 1.32(6H,d,J=6.6 Hz), 3.20-3.60(12H,m), 5.06(2H,s), 5.10-5.30(1H,m), 6.70-6.90(2H,m), 7.03(1H,d,J=9.6 Hz), 7.08(1H,d,J=2.6 Hz), 7.35-7.65(5H,m), 8.69(1H,d,J=7.5 Hz)

ESI/MS: 570[M+Na]⁺

### EXAMPLE 53

5-[2-(4-Benzyl-1-piperazinyl)-2-oxo]ethoxy-3-(3-oxo-2-isopropyl-2,3-dihydropyridazin-6-yl)-2-phenylpyrazolo[1,5-a]pyridine was prepared by similar procedure as that of Example 50.

mp: 128-129.5° C. (AcOEt)

NMR(DMSO-d6, δ): 1.31(6H,d,J=6.6 Hz), 2.20-2.50(4H,m), 3.30-3.60(4H,m), 5.00(2H,s), 5.05-5.30(1H,m), 6.70-6.90(2H,m), 7.03(1H,d,J=9.6 Hz), 7.14(1H,d,J=2.6 Hz), 7.20-7.40(5H,m), 8.69(1H,d,J=7.5 Hz)

ESI/MS: 563[M+H]⁺

### EXAMPLE 54

5-[2-(4-Acetyl-1-piperazinyl)-2-oxo]ethoxy-3-(3-oxo-2-isopropyl-2,3-dihydropyridazin-6-yl)-2-phenylpyrazolo[1,5-a]pyridine was prepared by similar procedure as that of Example 50.

mp: 170-171.5° C. (AcOEt —IPE)

NMR(DMSO-d6, δ): 1.32(6H,d,J=6.6 Hz), 2.04(3H,s), 3.30-3.65(8H, m), 5.05(2H,s), 5.10-5.35(1H,m), 6.75-6.90(2H,m), 7.03(1H,d, J=9.7 Hz), 7.18(1H,d,J=2.6 Hz), 7.35-7.65(5H,m), 8.70(1H,d, J=7.6 Hz)

ESI/MS: 537[M+Na]⁺

### EXAMPLE 55

To a solution of 5-[2-(1-piperazinyl)-2-oxo]ethoxy-3-(3-oxo-2-isopropyl-2,3-dihydropyridazin-6-yl)-2-phenylpyrazolo[1,5-a]pyridine hydrochloride (100 mg) in CH₂Cl₂(1.0 ml) was added triethylamine (0.0548 ml) at 0° C. To the mixture was added dropwise N,N-dimethylcarbamylchloride (0.0181 ml) in CH₂Cl₂ (1.0 ml) at 0° C. during 5 minutes, and the mixture was stirred at ambient temperature for 3 hours. To the reaction mixture was added water, and the mixture was stirred for 15 minutes, then the organic layer passed through the Presep (diatomaceous earth, granular) column with CH₂Cl₂, and evaporated. The residue was purified by silica-gel (4 g<30 V>) column chromatography (chloroform:MeOH=19:1) to give N,N-dimethyl-4-[{3-(3-oxo-2-isopropyl-2,3-dihydropyridazin-6-yl)-2-phenylpyrazolo[1,5-a]pyridin-5-yl}oxy]acetyl-1-piperazine carboxamide (53.4 mg).

mp: 151-152° C. (AcOEt-IPE)

NMR(DMSO-d6, δ): 1.32(6H,d,J=6.6 Hz), 2.77(6H,s), 3.00-3.60(8H, m), 5.03(2H,s), 5.05-5.35(1H,m), 6.75-6.90(2H,m), 7.03(1H,d, J=9.6 Hz), 7.17(1H,d,J=2.6 Hz), 7.35-7.65(5H,m), 8.70(1H,d, J=7.6 Hz)

ESI/MS: 566[M+Na]⁺

### EXAMPLE 56

N,N-Dimethyl-4-[{3-(3-oxo-2-isopropyl-2,3-dihydropyridazin-6-yl)-2-phenylpyrazolo[1,5-a]pyridin-5-yl}oxy]-1-piperidine carboxamide was prepared by similar procedure as that of Example 55.

mp: 164-166° C. (AcOEt)

NMR(DMSO-d6, δ): 1.35(6H,d,J=6.7 Hz), 1.55-1.85(2H,m), 1.90-2.20(2H,m), 2.75(6H,s), 2.85-3.15(2H,m), 3.25-3.60(2H,m), 4.55-4.75(1H,m), 5.10-5.35(1H,m), 6.65-6.90(2H,m), 7.01(1H,d, J=9.6 Hz), 7.30(1H,d,J=2.6 Hz), 7.40-7.65(5H,m), 8.69(1H,d, J=7.5 Hz)

ESI/MS: 523[M+Na]⁺

### EXAMPLE 57

5-[(1-Acety-4-piperidinyl)oxy]-3-(3-oxo-2-isopropyl-2,3-dihydropyridazin-6-yl)-2-phenylpyrazolo[1,5-a]pyridine was prepared by similar procedure as that of Example 55.

mp: 198-199° C. (AcOEt-IPE)

NMR(DMSO-d6, δ): 1.35(6H,d,J=6.7 Hz), 1.45-1.85(2H,m), 1.85-2.20(2H,m), 2.03(3H,s), 3.05-3.45(2H,m), 3.60-4.05(2H,m), 4.60-4.80(1H,m), 5.10-5.35(1H,m), 6.76(1H,dd,J=2.7, 7.6 Hz), 6.82(1H,d,J=9.7 Hz), 7.03(1H,d,J=9.6 Hz), 7.30(1H,d,J=2.6 Hz), 7.40-7.65(5H,m), 8.69(1H,d,J=7.5 Hz)

ESI/MS: 494[M+Na]⁺

### EXAMPLE 58

To the mixture of {[3-(3-oxo-2-isopropyl-2,3-dihydropyridazin-6-yl)-2-phenylpyrazolo[1,5-a]pyridin -yl]oxy}acetic acid (150 mg), WSC—HCl (85.3 mg), HOBt (60.1 mg) in DMF (1.0 ml) was added N,N-dimethylethylenediamine (0.0448 ml) at 0° C., and the mixture was stirred at ambient temperature for 1.5 hours. To the reaction mixture was added water, and the mixture was extracted with AcOEt, then the organic layer was washed with water twice and brine. Then the organic layer was passed through the Presep (diatomaceous earth, granular) column with AcOEt, and evaporated. The residue was purified by silica-gel (5 g) column chromatography (chloroform:MeOH=9:1) to give N-[2-(dimethylamino)ethyl]-2-{[3-(3-oxo-2-isopropyl-2,3-dihydropyridazin-6-yl)-2-phenylpyrazolo[1,5-a]pyridin-5-yl]oxy}acetamide (83.0 mg).

mp: 117-119° C. (AcOEt-IPE)

NMR(DMSO-d6, δ): 1.325(6H,d,J=6.6 Hz), 2.11(6H,s), 2.29(2H,t, J=6.7 Hz), 3.22(2H,q,J=6.3 Hz), 4.61(2H,s), 5.10-5.35(1H,m), 6.75-6.90(2H,m), 7.08(1H,d,J=9.7 Hz), 7.18(1H,d,J=2.6 Hz), 7.40-7.65(5H,m), 8.07(1H,t,J=5.6 Hz), 8.72(1H,d,J=7.5 Hz)

ESI/MS: 475[M+H]⁺

### EXAMPLE 59

2-{[3-(3-Oxo-2-isopropyl-2,3-dihydropyridazin-6-yl)-2-phenylpyrazolo[1,5-a]pyridin-5-yl]oxy}-N-(3-methoxybenzyl)acetamide was prepared by similar procedure as that of Example 58.

mp: 166-167.5° C. (AcOEt)

NMR(DMSO-d6, δ): 1.29(6H,d,J=6.6 Hz), 2.90(2H,t,J=7.2 Hz), 3.51(2H,q,J=6.5 Hz), 4.60(2H,s), 5.05-5.30(1H,m), 6.75-6.90(2H, m), 7.07(1H,d,J=9.7 Hz), 7.10-7.25(3H,m), 7.40-7.70(6H,m), 8.28(1H,t,J=5.8 Hz), 8.40-8.50(1H,m), 8.72(1H,d,J=7.5 Hz)

ESI/MS: 546[M+Na]⁺

### EXAMPLE 60

2-{[3-(3-oxo-2-isopropyl-2,3-dihydropyridazin-6-yl)-2-phenylpyrazolo[1,5-a)pyridin-5-yl]oxy}-N-[2-(2-pyridinyl)-ethyl]acetamide was prepared by similar procedure as that of Example 58.

mp: 188-190° C. (AcOEt)

NMR(DMSO-d6, δ): 1.30(6H,d,J=6.6 Hz), 2.90(3H,s), 4.32(2H,d, J=6.0 Hz), 4.70(2H,s), 5.05-5.30(1H,m), 6.70-6.90(5H,m), 7.00-7.25(3H,m), 7.40-7.65(5H,m), 8.65-8.80(2H,m)

ESI/MS: 531[M+Na]⁺

### EXAMPLE 61

N-Cyclopropyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 199-200° C. (AcOEt)

NMR(DMSO-d6, δ): 0.35-0.55(2H,m), 0.55-0.75(2H,m), 1.31(6H,d, J=6.6 Hz), 2.60-2.80(1H,m), 4.57(2H,s), 5.05-5.35(1H,m), 6.75-6.90(2H,m), 7.07(1H,d,J=9.6 Hz), 7.16(1H,d,J=2.6 Hz), 7.35-7.65(5H,m), 8.22(1H,d,J=4.0 Hz), 8.72(1H,d,J=7.5 Hz)

ESI/MS: 466[M+Na]⁺

### EXAMPLE 62

N-Cyclopentyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 187-188° C. (AcOEt)

NMR(DMSO-d6, δ): 1.30(6H,d,J=6.6 Hz), 1.30-1.95(8H,m), 3.95-4.20(1H,m), 4.58(2H,s), 5.05-5.35(1H,m), 6.75-6.95(2H,m), 7.09(1H,d,J=9.6 Hz), 7.15(1H,d,J=2.6 Hz), 7.35-7.65(5H,m), 8.06(1H,d,J=7.4 Hz), 8.72(1H,d,J=7.5 Hz)

ESI/MS: 494[M+Na]⁺

### EXAMPLE 63

N-Cyclohexyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 170-171.5° C. (AcOEt)

NMR(DMSO-d6, δ): 0.95-1.45(5H,m), 1.30(6H,d,J=6.6 Hz), 1.45-1.85(5H,m), 3.45-3.75(1H,m), 4.58(2H,s), 5.05-5.30(1H,m), 6.75-6.95(2H,m), 7.10(1H,d,J=9.6 Hz), 7.15(1H,d,J=2.6 Hz), 7.35-7.65(5H,m), 7.98(1H,d,J=8.0 Hz), 8.72(1H,d,J=7.5 Hz)

ESI/MS: 508[M+Na]⁺

### EXAMPLE 64

N-(4-Hydroxycyclohexyl)-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 146-148° C. (AcOEt)

NMR(DMSO-d6, δ): 1.05-1.45(4H,m), 1.29(6H,d,J=6.6 Hz), 1.60-1.90(4H,m), 3.40-3.70(1H,m), 4.53(1H,d,J=4.4 Hz), 4.57(2H,s), 5.05-5.35(1H,m), 6.75-6.90(2H,m), 7.10(1H,d,J=9.6 Hz), 7.14(1H, d,J=2.6 Hz), 7.35-7.65(5H,m), 7.95(1H,d,J=7.9 Hz), 8.72(1H,d, J=7.5 Hz)

Negative ESI/MS: 500[M−H]⁻

### EXAMPLE 65

N-Butyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 151-152° C. (AcOEt)

NMR(DMSO-d6, δ): 0.82(3H,t,J=7.2 Hz), 1.10-1.50(4H,m), 1.31(6H, d,J=6.6 Hz), 3.16(2H,q,J=6.4 Hz), 4.60(2H,s), 5.05-5.35(1H,m), 6.75-6.90(2H,m), 7.07(1H,d,J=9.6 Hz), 7.17(1H,d,J=2.6 Hz), 7.35-7.65(5H,m), 8.15(1H,d,J=5.6 Hz), 8.72(1H,d,J=7.5 Hz)

ESI/MS: 482[M+Na]⁺

### EXAMPLE 66

N-(2-Methoxyethyl)-2-[{3-(3-oxo-2-isopropyl-2,3-dihydropyridazin-6-yl)-2-phenylpyrazolo[1,5-a]pyridin-5-yl}oxy]acetamide was prepared by similar procedure as that of Example 58.

mp: 144.5-145.5° C. (AcOEt)

NMR(DMSO-d6, δ): 1.31(6H,d,J=6.6 Hz), 3.21(3H,s), 3.25-3.45(4H, m), 4.62(2H,s), 5.10-5.30(1H,m), 6.75-6.90(2H,m), 7.08(1H,d, J=9.6 Hz), 7.18(1H,d,J=2.6 Hz), 7.40-7.65(5H,m), 8.22(1H,t, J=5.2 Hz), 8.72(1H,d,J=7.5 Hz)

ESI/MS: 484[M+Na]⁺

### EXAMPLE 67

N-(2-Ethoxyethyl)-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 58.

mp: 126.5-127.5° C. (AcOEt)

NMR(DMSO-d6, δ): 1.05(3H,t,J=6.9 Hz), 1.31(6H,d,J=6.6 Hz), 3.15-3.50(6H,m), 4.62(2H,s), 5.05-5.30(1H,m), 6.75-6.95(2H,m), 7.07(1H,d,J=9.6 Hz), 7.18(1H,d,J=2.5 Hz), 7.40-7.65(5H,m), 8.20(1H,t,J=5.4 Hz), 8.72(1H,d,J=7.5 Hz)

ESI/MS: 498[M+Na]⁺

### EXAMPLE 68

To a mixture of 5-hydroxy-3-(3-oxo-2-isopropyl-2,3-dihydropyridazin-6-yl)-2-phenylpyrazolo[1,5-a]pyridine (1.00 g) and 2,6-lutidine (0.67 ml) in CH₂Cl₂ (20 ml) was added trifluoromethanesulfonic anhydride (0.73 ml) at 5° C. After stirring for 1.5 hours, the reaction mixture was concentrated and dissolved in AcOEt, washed with water, 1N HCl, water, saturated sodium hydrogen carbonate solution, water, and brine, dried over sodium sulfate, evaporated in vacuo. The residue was purified by silica gel column chromatography (n-hexane-AcOEt 3:2 elution) to give [3-(3-oxo-2-isopropyl-2,3-dihydropyridazin-6-yl)-2-phenylpyrazolo[1,5-a]pyridin-5-yl]-trifluoromethanesulfonate (1.31 g) as a solid.

¹H NMR (CDCl₃, δ): 1.49(6H,d,J=6.6 Hz), 5.44(1H,h,J=6.6 Hz), 6.76 (1H,d,J=9.7 Hz), 6.86(1H,dd,J=7.6, 2.8 Hz), 6.97(1H,d,J=9.7 Hz), 7.40-7.66(5H,m), 8.04(1H,dd,J=2.8, 0.5 Hz), 8.57(1H,dd,J=7.6, 0.5 Hz)

APCI/MS: 479[M+H]⁺

### EXAMPLE 69

A mixture of [3-(3-oxo-2-isopropyl-2,3-dihydropyridazin-6-yl)-2-phenylpyrazolo[1,5-a]pyridin-5-yl]-trifluoromethanesulfonate (800 mg), palladium(II) acetate (75 mg), 1,3-bis(diphenylphosphino)propane (138 mg), triethylamine (0.70 ml), and MeOH (4 ml) in DMF (8 ml) was stirred under carbon monoxide atmosphere at ambient temperature for 2 hours. The reaction mixture was diluted with AcOEt, washed with water, saturated sodium hydrogen carbonate solution, water (x 3), and brine, dried over magnesium sulfate, evaporated in vacuo. The residue was purified by silica gel column chromatography (CHCl₃-AcOEt 5:1 elution) to give methyl 3-(3-oxo-2-isopropyl-2,3-dihydropyridazin-6-yl)-2-phenylpyrazolo[1,5-a]pyridine-5-carboxylate (553.2 mg) as a solid.

mp: 186-187° C. (AcOEt)

IR (KBr): 1714, 1664, 1595, 1533, 1469, 1294 cm⁻¹

¹H NMR (CDCl₃, δ): 1.52(6H,d,J=6.6 Hz), 3.98(3H,s), 5.46(1H,h, J=6.6 Hz), 6.78(1H,d,J=9.7 Hz), 7.03(1H,d,J=9.7 Hz), 7.43-7.55(4H, m), 7.55-7.68(2H,m), 8.54(1H,dd,J=7.3, 0.9 Hz), 8.83(1H,dd, J=1.9, 0.9 Hz)

ESI/MS: 411[M+Na]⁺

### EXAMPLE 70

To a solution of methyl 3-(3-oxo-2-isopropyl-2,3-dihydropyridazin-6-yl)-2-phenylpyrazolo[1,5-a]pyridine-5-carboxylate (524 mg) in MeOH-THF (1:1, 15 ml) was added 1N NaOH solution (6.75 ml) and the mixture was stirred at ambient temperature for 1 hour. The organic solvent was evaporated, and THF was added. Then the mixture was acidified with 1N HCl under ice-cooling, and extracted with AcOEt. The organic layer was washed with water and brine, dried over magnesium sulfate, evaporated in vacuo to give 3-(3-oxo-2-isopropyl-2,3-dihydropyridazin-6-yl)-2-phenylpyrazolo[1,5-a]pyridine-5-carboxylic acid (438.2 mg) as a solid.

IR (KBr): 2927, 1703, 1643, 1574, 1535 cm⁻¹

¹HNMR (CDCl₃, δ): 1.35(6H,d,J=6.6 Hz), 5.25(1H,h,J=6.6 Hz), 6.89 (1H,d,J=9.6 Hz), 7.13(1H,d,J=9.6 Hz), 7.43(1H,dd,J=7.2, 1.8 Hz), 7.45-7.70(5H,m), 8.61(1H,dd,J=1.8, 0.8 Hz), 8.89(1H,dd,J=7.2, 0.8 Hz), 13.50(1H,br)

Negative ESI/MS: 373[M−H]⁻

### EXAMPLE 71

A mixture of [3-(3-oxo-2-isopropyl-2,3-dihydropyridazin-6-yl)-2-phenylpyrazolo [1,5-a]pyridin-5-yl]-trifluoromethanesulfonate (90 mg), tetrakis (triphenylphosphine)palladium(0) (33 mg), Zn(CN)₂ (45 mg), triethylamine (0.078 ml) in DMF was stirred at 80° C. for 31 hours. After cooling to ambient temperature, the reaction mixture was diluted with AcOEt, washed with saturated sodium hydrogen carbonate solution, water, and brine, dried over magnesium sulfate, evaporated in vacuo. The residue was purified by silica gel column chromatography (hexane-AcOEt 3:4 elution) to give 3-(3-oxo-2-isopropyl-2,3-dihydropyridazin-6-yl)-2-phenylpyrazolo[1,5-a]pyridine-5-carbonitrile (59.2 mg) as a solid.

mp: 202-203° C. (AcOEt)

IR (KBr): 2981, 2227, 1658, 1587 cm⁻¹

¹H NMR (CDCl₃, δ): 1.50(6H,d,J=6.6 Hz), 5.44(1H,h,J=6.6 Hz), 6.78 (1H,d,J=9.6 Hz), 6.99(1H,d,J=9.6 Hz), 7.02(1H,dd,J=7.2, 1.9 Hz), 7.40-7.66(5H,m), 8.39(1H,dd,J=1.9, 1.0 Hz), 8.59(1H,dd,J=7.2, 1.0 Hz)

APCI/MS: 356[M+H]⁺

### EXAMPLE 72

To a solution of 3-(3-oxo-2-isopropyl-2,3-dihydropyridazin-6-yl)-2-phenylpyrazolo[1,5-a]pyridine-5-carboxylic acid (50 mg) and 2-(1H-benzotriazol-1-yl)-1,1,3,3-tetramethyluronium tetrafluoroborate (TBTU) (86 mg) in DMF (2 ml) was added N,N-diisopropylethylamine (0.082 ml), and the mixture was stirred at ambient temperature for 10 minutes. To the mixture was added dimethylamine hydrochloride (22 mg), and stirred at the same temperature for 4 hours. The reaction mixture was diluted with AcOEt, washed with 0.1N HCl, water, saturated sodium hydrogen carbonate solution, water and brine, dried over magnesium sulfate, and evaporated in vacuo. The residue was purified by silica gal column chromatography (CH₂Cl₂—MeOH, 20:1 elution) to give N,N-dimethyl-3-(3-oxo-2-isopropyl-2,3-dihydropyridazin-6-yl)-2-phenylpyrazolo[1,5-a]pyridine-5-carboxamide (61.8 mg) as a solid.

IR (KBr): 2925, 1662, 1641, 1591, 1537, 1496, 1468, 1448, 1389 cm⁻¹

¹H NMR (CDCl₃, δ): 1.46(6H,d,J=6.6 Hz), 3.15(6H,s), 5.43(1H,h, J=6.6 Hz), 6.76(1H,d,J=9.6 Hz), 6.99(1H,dd,J=7.1, 1.8 Hz), 7.01 (1H,d,J=9.6 Hz), 7.40-7.68(5H,m), 8.06(1H,dd,J=1.8, 0.9 Hz), 8.55 (1H,dd,J=7.1, 0.9 Hz)

APCI/MS: 402[M+H]⁺

### EXAMPLE 73

To a mixture of 3-(3-oxo-2-isopropyl-2,3-dihydropyridazin-6-yl)-2-phenylpyrazolo[1,5-a]pyridine-5-carboxylic acid (50 mg) and pyrrolidine (0.015 ml) in DMF (2 ml) was added 1-hydroxybenzotriazole (27 mg) and 1-ethyl-3-(3′-dimethylaminopropyl)carbodiimide hydrochloride (51 mg). After stirring at ambient temperature for 24 hours, the reaction mixture was diluted with AcOEt, washed with 0.1N HCl, water, saturated sodium hydrogen carbonate solution, water and brine, dried over magnesium sulfate, and evaporated in vacuo. The residue was purified by silica gal column chromatography (CH₂Cl₂—MeOH, 20:1 elution) to give 5-(1-pyrrolidinylcarbonyl)-3-(3-oxo-2-isopropyl-2,3-dihydropyridazin-6-yl)-2-phenylpyrazolo[1,5-a]pyridine (56.2 mg) as a solid.

mp: 189-190° C. (AcOEt)

IR (KBr): 1668, 1616, 1595, 1531, 1469, 1404 cm⁻¹

¹H NMR (CDCl₃, δ): 1.46(6H,d,J=6.6 Hz), 1.85-2.10(4H,m), 3.48-3.77 (4H,m), 5.43(1H,h,J=6.6 Hz), 6.77(1H,d,J=9.6 Hz), 7.02(1H,d, J=9.6 Hz), 7.09(1H,dd,J=7.1, 1.8 Hz), 7.40-7.68(5H,m), 8.16(1H, s), 8.54(1H,d,J=7.1 Hz)

APCI/MS: 428[M+H]⁺

### EXAMPLE 74

5-[(4-Methyl-1-piperazinyl)carbonyl]-3-(3-oxo-2-isopropyl-2,3-dihydropyridazin-6-yl)-2-phenylpyrazolo[1,5-a]pyridine was prepared by similar procedure as that of Example 73.

IR (KBr): 1664, 1639, 1591, 1533, 1475 cm⁻¹

¹HNMR (CDCl₃, δ): 1.47(6H,d, J=6.6 Hz), 2.34(3H,s), 2.30-2.58(4H, m), 3.45-3.90(4H,m), 5.43(1H,h,J=6.6 Hz), 6.76(1H,d,J=9.6 Hz), 6.96(1H,dd,J=7.1, 1.8 Hz), 7.00(1H,d,J=9.6 Hz), 7.40-7.68(5H,m), 8.03(1H,dd,J=1.8, 0.9 Hz), 8.56(1H,dd,J=7.1, 0.9 Hz)

APCI/MS: 457[M+H]⁺

### EXAMPLE 75

N-(2-Hydroxyethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 3271, 1653, 1587, 1533 cm⁻¹

¹H NMR (CDCl₃, δ): 1.47(6H,d,J=6.6 Hz), 3.00-3.18(1H,m), 3.57-3.77 (2H,m), 3.77-3.97(2H,m), 5.36(1H,h,J=6.6 Hz), 6.61(1H,d, J=9.6 Hz), 6.94(1H,d,J=9.6 Hz), 7.25-7.65(6H,m), 8.40-8.58(2H,m)

APCI/MS: 418[M+H]⁺

### EXAMPLE 76

N-[2-(Dimethylamino)ethyl]-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 157-158° C. (AcOEt-n-Hexane)

IR (KBr): 3309, 1666, 1641, 1593, 1531 cm⁻¹

¹H NMR (CDCl₃, δ): 1.52(6H,d,J=6.6 Hz), 2.27(6H,s), 2.54(2H,t, J=5.8 Hz), 3.48-3.62(2H,m), 5.45(1H,h,J=6.6 Hz), 6.77(1H,d, J=9.6 Hz), 6.89-7.02(1H,m), 7.02(1H,d,J=9.6 Hz), 7.34(1H,dd, J=7.3, 2.0 Hz), 7.37-7.54(3H,m), 7.54-7.67(2H,m), 8.41-8.47(1H, m), 8.47-8.58(1H,m)

ESI/MS: 445[M+H]⁺

### EXAMPLE 77

3-(3-Oxo-2-isopropyl-2,3-dihydropyridazin-6-yl)-2-phenyl-N-[(1S)-1-phenylethyl]-pyrazolo[1,5-a]pyridine-5-carboxamide was prepared by similar procedure as that of Example 73.

IR (KBr): 3273, 2974, 1666, 1653, 1630, 1589, 1533 cm¹

¹H NMR (CDCl₃, δ): 1.42(6H,d,J=6.5 Hz), 1.64(3H,d,J=6.9 Hz), 5.25-5.48(2H,m), 6.66(1H,d,J=9.6 Hz), 6.67-6.83(1H,m), 6.95(1H, d,J=9.6 Hz), 7.24-7.65(11H,m), 8.42(1H,d,J=1.0 Hz), 8.51(1H, d, J=7.5 Hz)

Negative ESI/MS: 476[M−H]⁻

### EXAMPLE 78

N-(2-Ethoxyethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 149-150° C. (AcOEt)

IR (KBr): 3296, 2978, 1668, 1641, 1593, 1531 cm⁻¹

¹H NMR (CDCl₃, δ): 1.23(3H,t,J=7.0 Hz), 1.51(6H,d, J=6.6 Hz), 3.46-3.77(6H,m), 5.45(1H,h,J=6.6 Hz), 6.50-6.70(1H,m), 6.77(1H, d,J=9.6 Hz), 7.02(1H,d,J=9.6 Hz), 7.29(1H,dd,J=7.3, 2.0 Hz), 7.35-7.70(5H,m), 8.45-8.62(2H,m)

ESI/MS: 468[M+Na]⁺

### EXAMPLE 79

N-Methyl-N-[2-(2-pyridinyl)ethyl]-3-(3-oxo-2-isopropyl-2,3-dihydropyridazin-6-yl)-2-phenylpyrazolo [1,5-a]pyridine-5-carboxamide was prepared by similar procedure as that of Example 73.

IR (neat): 2979, 1655, 1631, 1591, 1533 cm⁻¹

¹H NMR (CDCl₃, δ): 1.43(6H,d,J=6.6 Hz), 2.85-3.32(5H,m), 3.75-4.05(2H,m), 5.41(1H,h,J=6.6 Hz), 6.55-6.65(14H,m)

ESI/MS: 515[M+Na]⁺

### EXAMPLE 80

5-[(4-Acetyl-1-piperazinyl)carbonyl]-3-(3-oxo-2-isopropyl-2,3-dihydropyridazin-6-yl)-2-phenylpyrazolo[1,5-a]pyridine was prepared by similar procedure as that of Example 73.

IR (KBr): 2978, 1649, 1587, 1533, 1475 cm⁻¹

¹HNMR (CDCl₃, δ): 1.45(6H,d,J=6.6 Hz), 2.15(3H,s), 3.40-3.90(8H, m), 5.43(1H,h,J=6.6 Hz), 6.77(1H,d,J=9.7 Hz), 6.95(1H,dd,J=7.1, 1.9 Hz), 7.01(1H,d,J=9.7 Hz), 7.35-7.68(5H,m), 7.98-8.10(1H,m), 8.58(1H,dd,J=7.1, 0.7 Hz)

ESI/MS: 507[M+Na]⁺

### EXAMPLE 81

5-[(cis-2,6-Dimethyl-4-morpholinyl)carbonyl]-3-(3-oxo-2-isopropyl-2,3-dihydropyridazin-6-yl)-2-phenylpyrazolo[1,5-a]pyridine was prepared by similar procedure as that of Example 73.

IR (KBr): 2976, 1664, 1591, 1533, 1475 cm⁻¹

¹H NMR (CDCl₃, δ): 1.02-1.37(6H,m), 1.46(6H,d,J=6.6 Hz), 2.50-4.70(6H,m), 5.44(1H,m,J=6.6 Hz), 6.77(1H,d,J=9.6 Hz), 6.97(1H,dd, J=7.1, 1.8 Hz), 7.01(1H,d,J=9.6 Hz), 7.38-7.65(5H,m), 8.04(1H,dd, J=1.7, 0.8 Hz), 8.57(1H,dd,J=7.1, 0.8 Hz)

ESI/MS: 494[M+Na]⁺

### EXAMPLE 82

N-Benzyl-N-methyl-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 2978, 1664, 1633, 1591, 1533, 1491 cm⁻¹

¹H NMR (CDCl₃, δ): 1.28-1.50(6H,m), 2.91-3.17(3H,m), 4.50-4.87(2H, m), 5.40(1H,h,J=6.6 Hz), 6.75(1H,d,J=9.6 Hz), 6.85-7.68(12H,m), 7.96-8.13(1H,m), 8.45-8.60(1H,m)

ESI/MS: 500[M+Na]⁺

### EXAMPLE 83

N-(tert-Butyl)-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 200-201° C. (AcOEt-Et₂O)

IR (KBr): 2970, 1678, 1657, 1589, 1531, 1506, 1460 cm⁻¹

¹H NMR (CDCl₃, δ): 1.50(9H,s), 1.52(6H,d,J=6.6 Hz), 5.47(1H,h, J=6.6 Hz), 6.01(1H,s), 6.76(1H,d,J=9.6 Hz), 7.02(1H,d,J=9.6 Hz), 7.33(1H,dd,J=7.2, 1.9 Hz), 7.38-7.67(5H,m), 8.36(1H,d,J=1.0 Hz), 8.53(1H,d,J=7.2 Hz)

Negative ESI/MS: 428[M−H]⁻

### EXAMPLE 84

5-[(4-Phenyl-1-piperidinyl)carbonyl]-3-(3-oxo-2-isopropyl-2,3-dihydropyridazin-6-yl)-2-phenylpyrazolo[1,5-a]-pyridine was prepared by similar procedure as that of Example 73.

mp: 184-185° C. (AcOEt-n-Hexane)

IR (KBr): 3054, 2856, 1666, 1630, 1595, 1477, 1412 cm⁻¹

¹H NMR (CDCl₃, δ): 1.46(6H,d,J=6.6 Hz), 1.46-2.08(6H,m), 2.71-3.27(3H,m), 5.43(1H,h,J=6.6 Hz), 6.76(1H,d,J=9.6 Hz), 6.99(1H,dd, J=7.0, 1.8 Hz), 7.01(1H,d,J=9.6 Hz), 7.12-7.65(10H,m), 8.00-8.08(1H,m), 8.56(1H,d,J=7.0 Hz)

ESI/MS: 540[M+Na]⁺

### EXAMPLE 85

N,N-Diethyl-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 157-158° C. (AcOEt)

IR (KBr): 2976, 1660, 1639, 1537, 1479 cm⁻¹

¹H NMR (CDCl₃, δ): 1.05-1.32(6H,m), 1.44(6H,d,J=6.6 Hz), 3.27-3.67(4H,m), 5.42(1H,h,J=6.6 Hz), 6.76(1H,d,J=9.6 Hz), 6.92(1H,dd, J=7.1, 1.9 Hz), 7.01(1H,d,J=9.6 Hz), 7.38-7.68(5H,m), 8.00(1H,dd, J=1.9, 0.8 Hz), 8.55(1H,dd,J=7.1, 0.8 Hz)

ESI/MS: 452[M+Na]⁺

### EXAMPLE 86

N-(2-Isopropoxyethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 124-125° C. (AcOEt)

IR (KBr): 3306, 2972, 1668, 1641, 1593, 1531 cm⁻¹

¹H NMR (CDCl₃₁ 8): 1.18(6H,d,J=6.1 Hz), 1.51(6H,d,J=6.6 Hz), 3.52-3.73(5H,m), 5.44(1H,h,J=6.6 Hz), 6.54-6.67(1H,m), 6.77(1H, d,J=9.6 Hz), 7.03(1H,d,J=9.6 Hz), 7.25(1H,dd,J=7.2, 2.0 Hz), 7.40-7.68(5H,m), 8.45-8.60(2H,m)

Negative ESI/MS: 458[M−H]⁻

### EXAMPLE 87

N-(3-Pyridinylmethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 192-193° C. (AcOEt)

IR (KBr): 3313, 1666, 1637, 1593, 1527, 1309 cm⁻¹

¹H NMR (CDCl₃, δ): 1.39(6H,d,J=6.6 Hz), 4.70(2H,d,J=5.7 Hz), 5.33(1H,h,J=6.6 Hz), 6.65(1H,d,J=9.6 Hz), 6.96(1H,d,J=9.6 Hz), 6.90-7.08(1H,m), 7.19-7.37(2H,m), 7.37-7.67(5H,m), 7.67-7.78(1H,m), 8.40-8.66(4H,m)

Negative ESI/MS: 463[M−H]⁻

### EXAMPLE 88

N-Methyl-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 225-226° C. (CHCl₃-Et₂O)

IR (KBr): 3359, 2972, 1651, 1587, 1558, 1539, 1468 cm⁻¹

¹H NMR (CDCl₃, δ): 1.50(6H,d,J=6.6 Hz), 3.07(3H,d,J=4.9 Hz), 5.44(1H,h,J=6.6 Hz), 6.18-6.38(1H,m), 6.76(1H,d,J=9.6 Hz), 7.02(1H,d,J=9.6 Hz), 7.24(1H,dd,J=7.3, 2.0 Hz), 7.35-7.70(5H,m), 8.40-8.57(2H,m)

Negative ESI/MS: 386[M−H]⁻

### EXAMPLE 89

5-(1-Piperidinylcarbonyl)-3-(3-oxo-2-isopropyl-2,3-dihydropyridazin-6-yl)-2-phenylpyrazolo[1,5-a]pyridine was prepared by similar procedure as that of Example 73.

mp: 185-186° C. (AcOEt)

IR (KBr): 2933, 2854, 1668, 1624, 1595, 1529, 1469 cm⁻¹

¹H NMR (CDCl₃, δ): 1.46(6H,d,J=6.6 Hz), 1.46-1.83(6H,m), 3.35-3.85(4H,m), 5.42(1H,h,J=6.6 Hz), 6.76(1H,d,J=9.6 Hz), 6.94(1H,dd,J=7.1, 1.7 Hz), 7.01(1H,d,J=9.6 Hz), 7.37-7.67(5H,m), 8.01(1H,dd, J=1.7, 0.9 Hz), 8.54(1H,dd,J=7.1, 0.9 Hz)

ESI/MS: 464[M+Na]⁺

### EXAMPLE 90

N-[2-(1-Pyrrolidinyl)ethyl]-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 2968, 1668, 1653, 1589, 1533 cm⁻¹

¹H NMR (CDCl₃, δ): 1.50(6H,d,J=6.6 Hz), 1.70-1.90(4H,m), 2.55-2.75(4H,m), 2.81(2H,t,J=5.7 Hz), 3.55-3.70(2H,m), 5.44(1H,h, J=6.6 Hz), 6.78(1H,d,J=9.6 Hz), 7.05(1H,d,J=9.6 Hz), 7.16-7.35(1H, m), 7.37(1H,dd,J=7.2, 2.0 Hz), 7.37-7.69(5H,m), 8.46-8.59(2H,m)

ESI/MS: 471[M+H]⁺

### EXAMPLE 91

N-(5-Methyl-1,3-thiazol-2-yl)-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 3238, 2970, 1641, 1581, 1562, 1537, 1462, 1304 cm⁻¹

¹H NMR (CDCl₃, δ): 1.41(6H,d,J=6.7 Hz), 2.32(3H,d,J=0.9 Hz), 5.33(1H,h,J=6.7 Hz), 6.72-6.88(2H,m), 7.03(1H,d,J=9.6 Hz), 7.39(1H,dd,J=7.3, 1.9 Hz), 7.40-7.70(5H,m), 8.49-8.75(2H,m), 12.12 (1H,br)

Negative ESI/MS: 469[M−H]⁻

### EXAMPLE 92

N-(2-Phenoxyethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 156-157° C. (AcOEt)

IR (KBr): 3336, 2979, 1666, 1631, 1591, 1533, 1496, 1466 cm⁻¹

¹H NMR (CDCl₃, δ):1.43(6H,d,J=6.6 Hz), 3.85-4.00(2H,m), 4.19(2H, t,J=4.9 Hz), 5.39(1H,h,J=6.6 Hz), 6.62-6.75(1H,m), 6.75(1H,d, J=9.6 Hz), 6.85-7.00(3H,m), 7.01(1H,d,J=9.6 Hz), 7.23-7.38(3H,m), 7.38-7.70(5H,m), 8.50(1H,d,J=1.4 Hz), 8.55 (1H,d,J=6.8 Hz)

Negative ESI/MS: 492[M−H]⁻

### EXAMPLE 93

N,N-Bis(2-methoxyethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 2978, 2929, 1658, 1637, 1591, 1535, 1477 cm⁻¹

¹HNMR (CDCl₃, δ): 1.45(6H,d,J=6.6 Hz), 3.10-3.88(14H,m), 5.42(1H, h, J=6.6 Hz), 6.77(1H,d,J19.6 Hz), 7.00(1H,dd,J=7.1, 1.8 Hz), 7.03(1H,d,J=9.6 Hz), 7.38-7.68(5H,m), 7.98-8.08(1H,m), 8.47-8.58(1H,m)

ESI/MS: 512[M+Na]⁺

### EXAMPLE 94

N-(1-Methyl-1-phenylethyl)-3-(3-oxo-2-isopropyl-2,3-dihydropyridazin-6-yl)-2-phenylpyrazolo[1,5-a]pyridine-5-carboxamide was prepared by similar procedure as that of Example 73.

IR (KBr): 3305, 2974, 1651, 1587, 1535, 1495 cm⁻¹

¹H NMR (CDCl₃, δ): 1.46(6H,d,J=6.6 Hz), 1.86(6H,s), 5.43(1H,h, J=6.6 Hz), 6.50(1H,br s), 6.75(1H,d,J=9.6 Hz), 7.01(1H,d,J=9.6 Hz), 7.20-7.68(11H,m), 8.35-8.42(1H,m), 8.46-8.57(1H,m)

Negative ESI/MS: 490[M−H]⁻

### EXAMPLE 95

N-Isopropyl-3-(3-oxo-2-isopropyl-2,3-dihydropyridazin-6-yl)-2-phenylpyrazolo[1,5-a]pyridine-5-carboxamide was prepared by similar procedure as that of Example 73.

mp: 230-232° C. (AcOEt)

IR (KBr): 3298, 2972, 1668, 1635, 1630, 1593, 1531 cm⁻¹

¹H NMR (CDCl₃, δ): 1.30(6H,d,J=6.5 Hz), 1.52(6H,d,J=6.6 Hz), 4.16-4.43(1H,m), 5.47(1H,h,J=6.6 Hz), 6.03(1H,d,J=7.7 Hz), 6.75(1H,d,J=9.6 Hz), 7.01(1H,d,J=9.6 Hz), 7.31(1H,dd,J=7.1, 2.0 Hz), 7.37-7.68(5H,m), 8.39-8.46(1H,m), 8.46-8.57(1H,m)

Negative ESI/MS: 414[M−H]⁻

### EXAMPLE 96

A mixture of [3-(3-oxo-2-isopropyl-2,3-dihydropyridazin-6-yl)-2-phenylpyrazolo[1,5-a]pyridin-5-yl]-trifluoromethanesulfonate (60 mg), 1-methylpiperazine (0.034 ml), Cs₂CO₃ (58 mg), Pd₂(dba)₃ (4.6 mg), BINAP (9.4 mg), and 18-crown-6 (3.3 mg) in toluene (1 ml) was stirred at 100° C. for 16 hours. After cooling to ambient temperature, the reaction mixture was diluted with AcOEt, washed with water and brine, dried over magnesium sulfate, evaporated in vacuo. The residue was purified by silica gel column chromatography (CH₂Cl₂-MeOH 10:1 elution) to give 5-(4-methyl-1-piperazinyl)-3-(3-oxo-2-isopropyl-2,3-dihydropyridazin-6-yl)-2-phenylpyrazolo[1,5-a]pyridine (37.6 mg) as a solid.

mp: 170-171° C. (AcOEt)

IR (KBr): 1658, 1643, 1587, 1535, 1448 cm⁻¹

¹H NMR (CDCl₃, δ): 1.49 (6H,d,J=6.6 Hz), 2.38(3H,s), 2.53-2.68 (4H,m), 3.25-3.40(4H,m), 5.45(1H,h,J=6.6 Hz), 6.67(1H,dd, J=7.7, 2.8 Hz), 6.70(1H,d,J=9.6 Hz), 6.95(1H,d,J=9.6 Hz), 7.30(1H,d, J=2.8 Hz), 7.36-7.65(5H,m), 8.31(1H,d,J=7.7 Hz)

APCI/MS: 429[M+H]⁺

### EXAMPLE 97

5-(1-Pyrrolidinyl)-3-(3-oxo-2-isopropyl-2,3-dihydropyridazin-6-yl)-2-pyrazolo [1,5-a]pyridine was prepared by similar procedure as that of Example 96.

mp: 165-167° C. (AcOEt-Et₂O)

IR (KBr): 1671, 1643, 1589, 1516 cm⁻¹

¹H NMR (CDCl₃, δ): 1.50(6H,d,J=6.6 Hz), 1.98-2.20(4H,m), 3.30-3.48(4H,m), 5.45(1H,h,J=6.6 Hz), 6.43(1H,dd,J=7.6, 2.7 Hz), δ 67 (1H,d,J=9.7 Hz), 6.95(1H,d,J=9.7 Hz), 6.95(1H,d,J=2.7 Hz), 7.37-7.66(5H,m), 8.27(1H,d,J=7.6 Hz)

ESI/MS: 400[M+H]⁺

### EXAMPLE 98

5-(4-Morpholinyl)-3-(3-oxo-2-isopropyl-2,3-dihydropyridazin-6-yl)-2-phenylpyrazolo[1,5-a]pyridine was prepared by similar procedure as that of Example 96.

mp: 182-187° C. (AcOEt)

IR (KBr): 1657, 1643, 1587, 1535, 1444 cm⁻¹

¹H NMR (CDCl₃, δ): 1.49(6H,d,J=6.6 Hz), 3.15-3.35(4H,m), 3.80-4.00 (4H,m), 5.45(1H,h,J=6.6 Hz), 6.57-6.70(1H,m), 6.71(1H,d,J=9.6 Hz), 6.96(1H,d,J=9.6 Hz), 7.29(1H,d,J=2.7 Hz), 7.35-7.70(5H,m), 8.34(1H,d,J=7.7 Hz)

APCI/MS: 416[M+H]⁺

### EXAMPLE 99

5-(4-Phenyl-1-piperazinyl)-3-(3-oxo-2-isopropyl-2,3-dihydropyridazin-6-yl)-2-phenylpyrazolo[1,5-a]pyridine was prepared by similar procedure as that of Example 96.

mp: >240° C. (AcOEt)

IR (KBr): 2814, 1662, 1645, 1591, 1535, 1487, 1446 cm⁻¹

¹H NMR (CDCl₃, δ): 1.51(6H,d,J=6.6 Hz), 3.25-3.53(8H,m), 5.46(1H, h,J=6.6 Hz), 6.62-6.77(2H,m), 6.85-7.65(12H,m), 8.27-8.38(1H,m)

ESI/MS: 491[M+H]⁺

### EXAMPLE 100

5-(cis-2,6-Dimethyl-4-morpholinyl)-3-(3-oxo-2-isopropyl-2,3-dihydropyridazin-6-yl)-2-phenylpyrazolo[1,5-a]-pyridine was prepared by similar procedure as that of Example 96.

mp: 176-177° C. (AcOEt-n-Hexane)

IR (KBr): 2974, 1647, 1585, 1537, 1448 cm⁻¹

¹H NMR (CDCl₃, δ): 1.28(6H,d, J=6.2 Hz), 1.51(6H,d,J=6.7 Hz), 2.43-2.63(2H,m), 3.43-3.62(2H,m), 3.68-3.93(2H,m), 5.46(1H,h, J=6.7 Hz), 6.67(1H,dd,J=7.6, 2.6 Hz), 6.70(1H,d,J=9.6 Hz), 6.95(1H, d,J=9.6 Hz), 7.32(1H,d,J=2.6 Hz), 7.33-7.66(5H,m), 8.32(1H,d, J=7.6 Hz)

ESI/MS: 444[M+H]⁺

### EXAMPLE 101

5-(4-Hydroxy-1-piperidinyl)-3-(3-oxo-2-isopropyl-2,3-dihydropyridazin-6-yl)-2-phenylpyrazolo[1,5-a]pyridine was prepared by similar procedure as that of Example 96.

mp: 213-215° C. (AcOEt)

IR (KBr): 3400, 2935, 1647, 1576, 1533, 1512, 1487 cm⁻¹

¹H NMR (CDCl₃, δ): 1.49(6H,d,J=6.6 Hz), 1.55-1.83(2H,m), 1.92-2.10(2H,m), 3.00-3.18(2H,m), 3.57-3.75(2H,m), 3.86-4.05(1H,m), 5.45(1H,h,J=6.6 Hz), 6.67(1H,dd,J=7.6, 2.7 Hz), 6.69(1H,d,J=9.6 Hz), 6.95(1H,d,J=9.6 Hz), 7.31(1H,d,J=2.7 Hz), 7.35-7.65(5H,m), 8.30(1H,d,J=7.6 Hz)

ESI/MS: 430[M+H]⁺

### EXAMPLE 102

5-[4-(2-Pyridinyl)-1-piperazinyl]-3-(3-oxo-2-isopropyl-2,3-dihydropyridazin-6-yl)-2-phenylpyrazolo[1,5-a]pyridine was prepared by similar procedure as that of Example 96.

mp: 221-222° C. (AcOEt)

IR (KBr): 2972, 2927, 2821, 1653, 1583, 1535, 1487 cm⁻¹

¹H NMR (CDCl₃, δ): 1.51(6H, d,J=6.7 Hz), 3.34-3.50(4H,m), 3.65-3.80(4H,m), 5.46(1H, h,J=6.7 Hz), 6.63-6.77(4H,m), 6.96(1H,d, J=9.6 Hz), 7.34(1H,d,J=2.5 Hz), 7.35-7.65(6H,m), 8.18-8.27(1H, m), 8.35(1H,d,J=7.6 Hz)

ESI/MS: 492[M+H]⁺

### EXAMPLE 103

5-[4-(2-Pyrimidinyl)-1-piperazinyl]-3-(3-oxo-2-isopropyl-2,3-dihydropyridazin-6-yl)-2-pyrazolo[1,5-a]pyridine was prepared by similar procedure as that of Example 96.

mp: >240° C. (AcOEt)

IR (KBr): 2972, 2925, 2817, 1653, 1585, 1543, 1504 cm⁻¹

¹H NMR (CDCl₃, δ): 1.51(6H,d, J=6.7 Hz), 3.30-3.46(4H,m), 3.95-4.08(4H,m), 5.46(1H,h,J=6.7 Hz), 6.57(1H,t,J=4.6 Hz), 6.70(1H,d, J=9.6 Hz), 6.73(1H,dd,J=7.7, 2.6 Hz), 6.95(1H,d,J=9.6 Hz), 7.33(1H,d,J=2.6 Hz), 7.36-7.65(5H,m), 8.35(1H,d,J=7.7 Hz), 8.37(2H,d,J=4.6 Hz)

ESI/MS: 493[M+H]⁺

### EXAMPLE 104

tert-Butyl 4-[3-(3-oxo-2-isopropyl-2,3-dihydropyridazin-6-yl)-2-phenylpyrazolo[1,5-a]pyridin-5-yl]-1-piperazinecarboxylate was prepared by similar procedure as that of Example 96.

mp: 163-164° C. (AcOEt)

IR (KBr): 2976, 1701, 1645, 1585, 1535, 1458, 1421 cm¹

¹HNMR (CDCl₃, δ): 1.50(6H,d,J=6.6 Hz), 1.50(9H,s), 3.20-3.33(4H, m), 3.55-3.67(4H,m), 5.45(1H,h,J=6.6 Hz), 6.66(1H,dd,J=7.7, 2.6 Hz), 6.70(1H,d,J=9.6 Hz), 6.95(1H,d,J=9.6 Hz), 7.28(1H,d,J=2.6 Hz), 7.37-7.63(5H,m), 8.33(1H,d,J=7.7 Hz)

ESI/MS: 515[M+H]⁺

### EXAMPLE 105

5-(4-Benzyl-1-piperazinyl)-3-(3-oxo-2-isopropyl-2,3-dihydropyridazin-6-yl)-2-phenylpyrazolo[1,5-a]pyridine was prepared by similar procedure as that of Example 96.

mp: 136-138° C. (AcOEt)

IR (KBr): 1647, 1581, 1537, 1516, 1491, 1454 cm⁻¹

¹H NMR (CDCl₃, δ): 1.47(6H,d,J=6.6 Hz), 2.54-2.70(4H,m), 3.22-3.38(4H,m), 3.59(2H,s), 5.43(1H,h,J=6.6 Hz), 6.60-6.74(2H,m), 6.94(1H,d,J=9.6 Hz), 7.20-7.64(11H,m), 8.30(1H,d,J=7.7 Hz)

ESI/MS: 505[M+H]⁺

### EXAMPLE 106

To a solution of 5-(4-hydroxy-1-piperidinyl)-3-(3-oxo-2-isopropyl-2,3-dihydropyridazin-6-yl)-2-phenylpyrazolo[1,5-a]pyridine (52.6 mg) in DMF (3 ml) was added NaH (60% oil suspension, 5.9 mg) at ambient temperature. After stirring for 10 minutes, iodomethane (0.038 ml) was added thereto. And the mixture was stirred for 5 hours at the same temperature. To the reaction mixture was added NaH (60% oil suspension, 5.9 mg) and iodomethane (0.038 ml), and stirred for 1.5 hours at 60° C. The reaction mixture was poured into ice-water, extracted with AcOEt, washed with water and brine, dried over sodium sulfate, evaporated in vacuo. The residue was purified by silica gel column chromatography (n-hexane-EtOAc 2:5 elution) to give 5-(4-methoxy-1-piperidinyl)-3-(3-oxo-2-isopropyl-2,3-dihydropyridazin-6-yl)-2-phenylpyrazolo[1,5-a]pyridine (13.9 mg) as a solid.

mp: 177-178° C. (AcOEt-Et₂O)

IR (KBr): 1658, 1643, 1587, 1533, 1514, 1462, 1425 cm⁻¹

¹H NMR (CDCl₃, δ): 1.49(6H,d,J=6.7 Hz), 1.55-2.10(4H,m), 3.04-3.24(2H,m), 3.40(3H,s), 3.35-3.70(3H,m), 5.45(1H,h,J=6.7 Hz), 6.67(1H,dd,J=7.7, 2.6 Hz), 6.69(1H,d,J=9.6 Hz), 6.95(1H,d,J=9.6 Hz), 7.30(1H,d,J=2.6 Hz), 7.35-7.65(5H,m), 8.29(1H,d,J=7.7 Hz)

ESI/MS: 444[M+H]⁺

### EXAMPLE 107

To a solution of tert-butyl 4-[3-(3-oxo-2-isopropyl-2,3-dihydropyridazin-6-yl)-2-phenylpyrazolo[1,5-a]pyridin-5-yl]-1-piperazinecarboxylate (557 mg) in CH₂Cl₂ (12 ml) was added 4N hydrogen chloride 1,4-dioxane solution (5.4 ml) at 5° C. After stirring at ambient temperature for 16 hours, saturated sodium hydrogen carbonate solution was added thereto, and the mixture was extracted with CHCl₃, washed with brine, dried over sodium sulfate, evaporated in vacuo. The residue was purified by silica gel column chromatography (CHCl₃-MeOH 10:1 elution) to give 5-(1-piperazinyl)-3-(3-oxo-2-isopropyl-2,3-dihydropyridazin-6-yl)-2-phenylpyrazolo[1,5-a]pyridine (417 mg) as a solid.

mp: 203-204° C. (AcOEt)

IR (KBr): 1643, 1583, 1535, 1514, 1489, 1448 cm⁻¹

¹H NMR (CDCl₃, δ): 1.49(6H,d,J=6.6 Hz), 3.00-3.13(4H,m), 3.20-3.33(4H,m), 5.45(1H,h,J=6.6 Hz), 6.68(1H,dd,J=7.7, 2.6 Hz), 6.70(1H,d,J=9.6 Hz), 6.95(1H,d,J=9.6 Hz), 7.29(1H,d,J=2.6 Hz), 7.36-7.65(5H,m), 8.31(1H,d,J=7.7 Hz)

ESI/MS: 415[M+H]⁺

### EXAMPLE 108

To a solution of 5-(1-piperazinyl)-3-(3-oxo-2-isopropyl-2,3-dihydropyridazin-6-yl)-2-phenylpyrazolo[1,5-a]pyridine (50 mg) and triethylamine (0.022 ml) in CH₂Cl₂ (1.5 ml) was added methyl chlorocarbonate (0.010 ml) at 5° C. After stirring at ambient temperature for 1 hour, the reaction mixture was diluted with AcOEt, washed with saturated sodium hydrogen carbonate solution, water, and brine, dried over sodium sulfate, evaporated in vacuo. The residue was purified by silica gel column chromatography (n-hexane-AcOEt 1:10 elution) to give methyl 4-[3-(3-oxo-2-isopropyl-2,3-dihydropyridazin-6-yl)-2-phenylpyrazolo[1,5-a]pyridin-5-yl]-1-piperazinecarboxylate (55.2 mg) as a solid.

mp: 206-207° C. (AcOEt)

IR (KBr): 1707, 1651, 1583, 1537, 1475 cm⁻¹

¹H NMR (CDCl₃, δ): 1.49(6H,d,J=6.6 Hz), 3.20-3.35(4H,m), 3.60-3.75(4H,m), 3.76(3H,s), 5.45(1H,h,J=6.6 Hz), 6.61-6.75(2H,m), 6.95(1H,d,J=9.7 Hz), 7.29(1H,d,J=2.6 Hz), 7.38-7.65(5H,m), 8.34(1H,d,J=7.7 Hz)

ESI/MS: 473[M+H]⁺

### EXAMPLE 109

4-[3-(3-oxo-2-isopropyl-2,3-dihydropyridazin-6-yl)-2-phenylpyrazolo[1,5-a]pyridin-5-yl]-N,N-dimethyl-1-piperazinecarboxamide was prepared by similar procedure as that of Example 108.

mp: 160-161° C. (AcOEt)

IR (KBr): 1647, 1583, 1535, 1491, 1456, 1392 cm⁻¹

¹H NMR (CDCl₃, δ): 1.49(6H,d,J=6.6 Hz), 2.89(6H,s), 3.22-3.47(8H, m), 5.45(1H,h,J=6.6 Hz), 6.67(1H,dd,J=7.7, 2.7 Hz), 6.71(1H,d, J=9.6 Hz), 6.95(1H,d,J=9.6 Hz), 7.28(1H,d,J=2.7 Hz), 7.37-7.65(5H, m), 8.33(1H,d,J=7.7 Hz)

ESI/MS: 508[M+Na]⁺

### EXAMPLE 110

5-[4-(Methylsulfonyl)-1-piperazinyl]-3-(3-oxo-2-isopropyl-2,3-dihydropyridazin-6-yl)-2-phenylpyrazolo[1,5-a]pyridine was prepared by similar procedure as that of Example 108.

mp: 219-220° C. (AcOEt)

IR (KBr): 2979, 2837, 1655, 1583, 1539, 1452 cm⁻¹

¹H NMR (CDCl₃, δ): 1.49(6H,d,J=6.6 Hz), 2.86(3H,s), 3.35-3.50(8H, m), 5.49(1H,h,J=6.6 Hz), 6.65(1H,dd,J=7.7, 2.8 Hz), 6.71(1H,d, J=9.6 Hz), 6.95(1H,d,J=9.6 Hz), 7.33(1H,d,J=2.8 Hz), 7.37-7.64(5H, m), 8.36(1H,d,J=7.7 Hz)

ESI/MS: 515[M+Na]⁺

### EXAMPLE 111

5-[4-(Phenylsulfonyl)-1-piperazinyl]-3-(3-oxo-2-isopropyl-2,3-dihydropyridazin-6-yl)-2-phenylpyrazolo[1,5-a]pyridine was prepared by similar procedure as that of Example 108.

mp: 219-220° C. (AcOEt)

IR (KBr): 2970, 2862, 1649, 1587, 1539, 1450 cm⁻¹

¹H NMR (CDCl₃, δ): 1.49(6H,d,J=6.6 Hz), 3.12-3.44(8H,m), 5.46(1H, h,J=6.6 Hz), 6.56(1H,dd,J=7.7, 2.8 Hz), 6.70(1H,d,J=9.6 Hz), 6.93(1H,d,J=9.6 Hz), 7.27(1H,d,J=2.8 Hz), 7.36-7.70(8H,m), 7.70-7.88(2H,m), 8.30(1H,d,J=7.7 Hz)

ESI/MS: 555[M+H]⁺

### EXAMPLE 112

5-(4-Benzoyl-1-piperazinyl)-3-(3-oxo-2-isopropyl-2,3-dihydropyridazin-6-yl)-2-phenylpyrazolo[1,5-a]pyridine was prepared by similar procedure as that of Example 108.

mp: 124-126° C. (AcOEt-n-Hexane)

IR (KBr): 2978, 2833, 1658, 1639, 1583, 1533, 1514, 1460, 1421 cm⁻¹

¹HNMR (CDCl₃, δ): 1.48(6H,d, J=6.6 Hz), 3.18-4.05(8H,m), 5.45(1H, h,J=6.6 Hz), 6.66(1H,dd,J=7.7, 2.6 Hz), 6.71(1H,d,J=9.7 Hz), 6.95(1H,d,J=9.7 Hz), 7.30(1H,d,J=2.6 Hz), 7.34-7.65(10H,m), 8.35(1H,d,J=7.7 Hz)

ESI/MS: 541[M+Na]⁺

### EXAMPLE 113

5-(4-Acetyl-1-piperazinyl)-3-(3-oxo-2-isopropyl-2,3-dihydropyridazin-6-yl)-2-phenylpyrazolo[1,5-a]pyridine was prepared by similar procedure as that of Example 108.

mp: 148-149° C. (AcOEt-n-Hexane)

IR (KBr): 1643, 1583, 1535, 1444, 1423 cm⁻¹

¹HNMR (CDCl₃, δ): 1.49(6H,d,J=6.7 Hz), 2.17(3H,s), 3.19-3.38(4H, m), 3.57-3.88(4H,m), 5.46(1H,h,J=6.7 Hz), 6.66(1H,dd,J=7.7, 2.6 Hz), 6.71(1H,d,J=9.6 Hz), 6.95(1H,d,J=9.6 Hz), 7.29(1H,d, J=2.6 Hz), 7.36-7.64(5H,m), 8.35(1H,d,J=7.7 Hz)

ESI/MS: 479[M+Na]⁺

### EXAMPLE 114

To a mixture of 5-(1-piperazinyl)-3-(3-oxo-2-isopropyl-2,3-dihydropyridazin-6-yl)-2-phenylpyrazolo[1,5-a]pyridine (45 mg), N,N-dimethylglycine hydrochloride (18.2 mg), and N,N-diisopropylethylamine (0.023 ml) in DMF (1 ml) was added 1-hydroxybenzotriazole (22 mg) and 1-ethyl-3-(3′-dimethylaminopropyl)carbodiimide hydrochloride (31.3 mg). After stirring at ambient temperature for 4 hours, the reaction mixture was diluted with AcOEt, washed with saturated sodium hydrogen carbonate solution, water and brine, dried over magnesium sulfate, and evaporated in vacuo. The residue was purified by silica gal column chromatography (CHCl₃-MeOH, 10:1 elution) to give 5-{4-[(dimethylamino)acetyl]-1-piperazinyl}-3-(3-oxo-2-isopropyl-2,3-dihydropyridazin-6-yl)-2-phenylpyrazolo[1,5-a]pyridine (52.2 mg) as a solid.

mp: 129-130° C. (AcOEt)

IR (KBr): 1647, 1585, 1537, 1458, 1423 cm⁻¹

¹H NMR (CDCl₃, δ): 1.49(6H,d,J=6.6 Hz), 2.30(6H,s), 3.17(2H,s), 3.18-3.38(4H,m), 3.73-3.90(4H,m), 5.46(1H,h,J=6.6 Hz), 6.67(1H, dd,J=7.7, 2.7 Hz), 6.70(1H,d,J=9.6 Hz), 6.95(1H,d,J=9.6 Hz), 7.29(1H,d,J=2.7 Hz), 7.37-7.65(5H,m), 8.34(1H,d,J=7.7 Hz)

ESI/MS: 500[M+H]⁺

### EXAMPLE 115

5-Methoxy-3-(3-oxo-2-methyl-2,3-dihydropyridazin-6-yl)-2-phenylpyrazolo[1,5-a]pyridine was prepared by similar procedure as that of Example 2.

mp: 183-184° C. (AcOEt)

NMR (CDCl₃, δ): 3.91(3H,s), 3.92(3H,s), 6.60(1H,dd,J=7.5, 2.8 Hz), 6.75(1H,d,J=9.7 Hz), 6.98(1H,d,J=9.7 Hz), 7.30(1H,d,J=2.8 Hz), 7.41-7.47(3H,m), 7.54-7.61(2H,m), 8.34(1H,dd,J=7.5, 0.4 Hz)

APCI/MS: 333[M+H]⁺

Anal.Calcd for C₁₉H₁₆N₄O₂: C, 68.66; H, 4.85; N, 16.86 Found: C, 68.29; H, 4.76; N, 16.62

### EXAMPLE 116

5-Methoxy-3-(3-oxo-2-ethyl-2,3-dihydropyridazin-6-yl)-2-phenylpyrazolo[1,5-a]pyridine was prepared by similar procedure as that of Example 2.

mp: 148-149° C. (AcOEt)

NMR (CDCl₃, δ): 1.51 (3H,t,J=7.2 Hz), 3.91 (3H,s), 4.34 (2H,q, J=7.2 Hz), 6.60 (1H,dd,J=7.5, 2.8 Hz), 6.74 (1H,d,J=9.7 Hz), 6.98 (1H,d,J=9.7 Hz), 7.32(1H,d,J=2.8 Hz), 7.41-7.47(3H,m), 7.56-7.62 (2H,m), 8.34(1H,d,J=7.5 Hz)

APCI/MS: 347 [M+H]⁺

Anal.Calcd for C₂₀H₁₈N₄O₂: C, 69.35; H, 5.24; N, 16.17 Found: C, 69.67; H, 5.23; N, 16.27

### EXAMPLE 117

5-Methoxy-3-(3-oxo-2-propyl-2,3-dihydropyridazin-6-yl)-2-phenylpyrazolo[1,5-a]pyridine was prepared by similar procedure as that of Example 2.

mp: 174° C. (AcOEt)

NMR (CDCl₃, δ): 1.06 (3H,t,J=7.4 Hz), 1.89-2.08 (2H,m), 3.90 (3H, s), 4.25 (2H,t,J=7.2 Hz), 6.60 (1H,dd, J=7.5, 2.8 Hz), 6.74 (1H,d, J=9.7 Hz), 6.97 (1H,d,J=9.7 Hz), 7.30 (1H,d,J=2.7 Hz), 7.41-7.47 (3H,m), 7.55-7.62(2H,m), 8.33(1H,d,J=7.5 Hz)

APCI/MS: 361 [M+H]⁺

Anal.Calcd for C₂₁H₂₀N₄O₂: C, 69.98; H, 5.59; N, 15.55 Found: C, 70.00; H, 5.52; N, 15.46

### EXAMPLE 118

5-Methoxy-3-[3-oxo-2-(tetrahydrofuran-3-yl)-2,3-dihydropyridazin-6-yl]-2-phenylpyrazolo[1,5-a]pyridine was prepared by similar procedure as that of Example 2.

mp: 207-208° C. (AcOEt)

NMR (CDCl₃, δ): 2.28-2.58(2H,m), 3.87-3.98(1H,m), 3.94(3H,s), 4.06-4.30(3H,m), 5.80-5.91(1H,m), 6.60(1H,dd,J=7.5, 2.7 Hz), 6.70(1H,d,J=9.7 Hz), 6.98(1H,d,J=9.7 Hz), 7.42-7.51(4H,m), 7.55-7.61(2H,m), 8.33(1H,d,J=7.5 Hz)

APCI/MS: 389 [M+H]⁺

Anal.Calcd for C₂₂H₂₀N₄O₃: C, 68.03; H, 5.19; N, 14.42 Found: C, 68.26; H, 5.15; N, 14.44

### EXAMPLE 119

5-Methoxy-3-[3-oxo-2-((3R)-tetrahydrofuran-3-yl)-2,3-dihydropyridazin-6-yl]-2-phenylpyrazolo[1,5-a]pyridine was prepared by similar procedure as that of Example 2.

mp: 194-195° C. (AcOEt)

NMR (CDCl₃, δ): 2.28-2.58(2H,m), 3.87-3.98(1H,m), 3.95(3H,s), 4.06-4.27(3H,m), 5.80-5.91(1H,m), 6.60(1H,dd,J=7.5, 2.7 Hz), 6.70(1H,d,J=9.7 Hz), 6.98(1H,d,J=9.7 Hz), 7.44-7.51(4H,m), 7.55-7.61(2H,m), 8.33(1H,d,J=7.5 Hz)

APCI/MS: 389 [M+H]⁺

Anal.Calcd for C₂₂H₂₁N₄O₃: C, 68.03; H, 5.19; N, 14.42 Found: C, 68.06; H, 5.14; N, 14.38

### EXAMPLE 120

5-Methoxy-3-[3-oxo-2-((3S)-tetrahydrofuran-3-yl)-2,3-dihydropyridazin-6-yl]-2-phenylpyrazolo[1,5-a]pyridine was prepared by similar procedure as that of Example 2.

mp: 194-195° C. (AcOEt)

NMR (CDCl₃, δ): 2.28-2.58(2H,m), 3.87-3.98(1H,m), 3.95(3H,s), 4.06-4.27(3H,m), 5.80-5.91(1H,m), 6.60(1H,dd,J=7.5, 2.7 Hz), 6.70(1H,d,J=9.7 Hz), 6.98(1H,d,J=9.7 Hz), 7.44-7.51(4H,m), 7.55-7.61(2H,m), 8.33(1H,d,J=7.5 Hz)

APCI/MS: 389 [M+H]⁺

Anal.Calcd for C₂₂H₂₀N₄O₃: C, 68.03; H, 5.19; N, 14.42 Found: C, 67.85; H, 5.14; N, 14.33

### EXAMPLE 121

5-Methoxy-3-[3-oxo-2-(1-methoxy-2-propyl)-2,3-dihydropyridazin-6-yl]-2-phenylpyrazolo[1,5-a]pyridine was prepared by similar procedure as that of Example 2.

mp: 138-139° C. (AcOEt)

NMR (CDCl₃, δ): 1.47(3H,d,J=6.8 Hz), 3.37(3H,s), 3.59(1H,dd, J=10.1, 5.2 Hz), 3.90(3H,s), 3.95(1H,dd,J=10.1, 8.0 Hz), 5.52-5.59(1H,m), 6.60(1H,dd,J=7.5, 2.7 Hz), 6.74(1H,d,J=9.7 Hz), 6.97 (1H,d,J=9.7 Hz), 7.37(1H,d,J=2.7 Hz), 7.43-7.48(3H,m), 7.58-7.62 (2H,m), 8.33(1H,d,J=7.5 Hz)

APCI/MS: 391 [M+H]⁺

### EXAMPLE 122

A mixture of 5-methoxy-3-(3-oxo-2,3-dihydropyridazin-6-yl)-2-phenylpyrazolo[1,5-a]pyridine (274 mg), 4-hydroxy-1-methylpiperidine (159 mg), diethyl azodicarboxylate (299 mg) and triphenylphosphine (451 mg) in tetrahydrofuran (5.5 ml) was stirred at ambient temperature for 18 hours. After the solvent was removed under reduced pressure, the residue was dissolved in ethyl acetate and the solution was extracted with 6 N hydrochloric acid. Potassium carbonate was added to the aqueous solution to adjust pH to 9 and extracted twice with ethyl acetate. The combined extracts were washed with water and brine, dried over anhydrous magnesium sulfate, and concentrated under reduced pressure to give crude material, which was then purified by silica gel column chromatography using a mixture of chloroform and methanol (50:1) to give 5-methoxy-3-[3-oxo-2-(1-methylpiperidin-4-yl)-2,3-dihydropyridazin-6-yl]-2-phenylpyrazolo[1,5-a]pyridine.

mp: 154-155° C. (AcOEt-Diisopropyl ether)

NMR (DMSO-d6, δ):1.84-2.10(6H,m), 2.20(3H,s), 2.86-2.89(2H,m), 3.89(3H,s), 4.79-4.82(1H,m), 6.77(1H,dd,J=7.5, 2.7 Hz), 6.85(1H, d,J=9.6 Hz), 7.07(1H,d,J=9.6 Hz), 7.32(1H,d,J=2.7 Hz), 7.45-7.60 (5H,m), 8.69(1H,d,J=7.5 Hz)

APCI/MS: 416 [M+H]⁺

### EXAMPLE 123

5-Methoxy-3-[3-oxo-2-(1-methylpiperidin-3-yl)-2,3-dihydropyridazin-6-yl]-2-phenylpyrazolo[1,5-a]pyridine was prepared by similar procedure as that of Example 122.

mp: 179-180° C. (AcOEt-Diisopropyl ether)

NMR(DMSO-d6, δ): 1.63-1.89(6H,m), 2.19(3H,s), 2.75-2.80(1H,m), 2.92-2.97(1H,m), 3.91(3H,s), 4.93(1H,m), 6.76(1H,dd,J=7.5, 2.7 Hz), 6.85(1H,d,J=9.7 Hz), 7.07(1H,d,J=9.7 Hz), 7.46-7.56(5H, m), 8.69(1H,d,J=7.5 Hz)

APCI/MS: 416 [M+H]⁺

### EXAMPLE 124

5-Methoxy-3-[3-oxo-2-(tetrahydropyran-4-yl)-2,3-dihydropyridazin-6-yl]-2-phenylpyrazolo[1,5-a]pyridine was prepared by similar procedure as that of Example 122.

mp: 224-225° C. (AcOEt)

NMR (CDCl₃, δ): 1.95 (2H,dd,J=12.3, 2.4 Hz), 2.16-2.37(2H,m), 3.64 (2H,t,J=11.3 Hz), 3.93(3H,s), 4.12(2H,dd,J=11.3, 4.4 Hz), 5.23-5.38(1H,m), 6.62(1H,dd,J=7.5, 2.7 Hz), 6.73(1H,d,J=9.7 Hz), 6.99 (1H,d,J=9.7 Hz), 7.42-7.47(4H,m), 7.57-7.52(2H,m), 8.35(1H,d, J=7.5 Hz)

APCI/MS: 403 [M+H]⁺

### EXAMPLE 125

5-Methoxy-3-(3-oxo-2,3-dihydropyridazin-6-yl)-2-(2-fluorophenyl)pyrazolo[1,5-a]pyridine was prepared by similar procedure as that of Example 1.

NMR (DMSO-d6, δ): 3.88(3H,s), 6.76-6.84(2H,m), 7.12(1H,d, J=9.8 Hz), 7.24-7.38(3H,m), 7.49-7.68(2H,m), 8.70(1H,d,J=7.5 Hz), 13.0(1H,brd s)

APCI/MS: 337 [M+H]⁺

### EXAMPLE 126

5-Methoxy-3-(3-oxo-2-methyl-2,3-dihydropyridazin-6-yl)-2-(2-fluorophenyl)pyrazolo[1,5-a]pyridine was prepared by similar procedure as that of Example 2.

mp: 220.5-221.5° C. (95% EtOH)

NMR (DMSO-d6, δ): 3.71(3H,s), 3.92(3H,s), 6.80(1H,dd,J=7.6, 2.8 Hz), 6.85(1H,d,J=9.7 Hz), 7.05(1H,d,J=9.7 Hz), 7.29-7.40(3H, m), 7.51-7.68(2H,m), 8.70(1H,d,J=7.6 Hz)

APCI/MS: 351 [M+H]⁺

Anal.Calcd for C₁₉H₁₅FN₄O₂: C, 65.14; H, 4.32; N, 15.99 Found: C, 65.20; H, 4.22; N, 15.93

### EXAMPLE 127

5-Methoxy-3-(3-oxo-2-isopropyl-2,3-dihydropyridazin-6-yl)-2-(2-fluorophenyl)pyrazolo[1,5-a]pyridine was prepared by similar procedure as that of Example 2.

mp: 174-175° C. (AcOEt-Diisopropyl Ether)

NMR (DMSO-d6, δ): 1.23(6H,d,J=6.6 Hz), 3.90(3H,s), 5.10-5.23(1H, m), 6.79(1H,dd,J=7.5, 2.7 Hz), 6.86(1H,d,J=9.7 Hz), 7.18(1H,d, J=9.7 Hz), 7.27-7.39(3H,m), 7.49-7.68(2H,m), 8.71 (1H,d, J=7.5 Hz)

APCI/MS: 379 [M+H]⁺

Anal.Calcd for C₂₁H₁₉FN₄O₂: C, 66.66; H, 5.06; N, 14.81 Found: C, 66.52; H, 5.02; N, 14.70

### EXAMPLE 128

5-Methoxy-3-(3-oxo-2,3-dihydropyridazin-6-yl)-2-(4-fluorophenyl)pyrazolo[1,5-a]pyridine was prepared by similar procedure as that of Example 1.

NMR (DMSO-d6, δ): 4.05(3H,s), 6.74(1H,dd,J=7.5, 2.7 Hz), 6.83(1H, dd,J=9.8, 2.0 Hz), 7.10-7.15(2H,m), 7.30(2H,t,J=8.8 Hz), 7.57-7.64(2H,m), 8.67(1H,d,J=7.5 Hz), 13.0(1H,brd s)

APCI/MS: 337 [M+H]⁺

### EXAMPLE 129

5-Methoxy-3-(3-oxo-2-isopropyl-2,3-dihydropyridazin-6-yl)-2-(4-fluorophenyl)pyrazolo[1,5-a]pyridine was prepared by similar procedure as that of Example 2.

mp: 176-177° C. (AcOEt-Diisopropyl Ether)

NMR (DMSO-d6, δ): 1.33(6H,d,J=6.6 Hz), 3.87(3H,s), 5.22(1H,m), 6.76(1H,dd,J=7.6, 2.8 Hz), 6.86(1H,d,J=9.6 Hz), 7.11(1H,d, J=9.6 Hz), 7.25-7.36(3H,m), 7.57-7.64(2H,m), 8.68(1H,d,J=7.6 Hz)

APCI/MS: 379 [M+H]⁺

### EXAMPLE 130

5-Methoxy-3-(3-oxo-2-methyl-2,3-dihydropyridazin-6-yl)-2-(4-fluorophenyl)pyrazolo[1,5-a]pyridine was prepared by similar procedure as that of Example 2.

mp: 221-222° C. (AcOEt)

NMR (DMSO-d6, δ): 3.75(3H,s), 3.89(3H,s), 6.75(1H,dd,J=7.6, 2.8 Hz), 6.87(1H,d,J=9.6 Hz), 7.08(1H,d,J=9.6 Hz), 7.27-7.35(3H, m), 7.59-7.66(2H,m), 8.67(1H,d,J=7.6 Hz)

APCI/MS: 351 [M+H]⁺

### EXAMPLE 131

5-Methoxy-3-(3-oxo-2-ethyl-2,3-dihydropyridazin-6-yl)-2-(4-fluorophenyl)pyrazolo[1,5-a]pyridine was prepared by similar procedure as that of Example 2.

mp: 179-180° C. (AcOEt-Diisopropyl Ether)

NMR (DMSO-d6, δ): 1.33(3H,t,J=7.2 Hz), 3.88(3H,s), 4.16(2H,q, J=7.2 Hz), 6.76(1H,dd,J=7.5, 2.8 Hz), 6.87(1H,d,J=9.6 Hz), 7.11 (1H,d,J=9.6 Hz), 7.24-7.35(3H,m), 7.59-7.66(2H,m), 8.68(1H,d, J=7.5 Hz)

APCI/MS: 365 [M+H]⁺

### EXAMPLE 132

5-Methoxy-3-(3-oxo-2-propyl-2,3-dihydropyridazin-6-yl)-2-(4-fluorophenyl)pyrazolo[1,5-a]pyridine was prepared by similar procedure as that of Example 2.

mp: 190-191° C. (AcOEt-Diisopropyl Ether)

NMR (DMSO-d6, δ): 0.93(3H,t,J=7.4 Hz), 1.71-1.89(2H,m), 3.87(3H, s), 4.10(2H,t,J=7.0 Hz), 6.76(1H,dd,J=7.5, 2.7 Hz), 6.87(1H,d, J=9.7 Hz), 7.10(1H,d,J=9.7 Hz), 7.22(1H,d,J=2.7 Hz), 7.30(2H,t, J=7.9 Hz), 7.58-7.65(2H,m), 8.67(1H,d,J=7.5 Hz)

APCI/MS: 379 [M+H]⁺

### EXAMPLE 133

The mixture of 7-amino-3-(6-methoxy-3-pyridazinyl)-2-phenylpyrazolo[1,5-a]pyridine (87 mg) and conc.HCl (1 ml) in EtOH (2 ml) was heated with stirring at 80° C. for 17 hours. The mixture was made basic with sodium hydrogen carbonate solution. The resultant precipitate was collected by filtration to give 7-amino-3-(3-oxo-2,3-dihydropyridazin-6-yl)-2-phenylpyrazolo[1,5-a]pyridine (85 mg).

mp: 157-159° C.

NMR (DMSO-d6, δ): 6.17-6.25(1H,m), 6.78(1H,d,J=9.8 Hz), 6.88-7.28(5H,m), 7.46-7.48(3H,m), 7.60-7.63(2H,m), 13.04(1H,s)

ESI/MS: 304 [M+H]⁺

### EXAMPLE 134

7-Amino-3-(3-oxo-2-isopropyl-2,3-dihydropyridazin-6-yl)-2-phenylpyrazolo[1,5-a]pyridine was prepared by similar procedure as that of Example 2.

mp: 218-219° C.

NMR (DMSO-d6, δ): 1.29(6H,d,J=6.6 Hz), 5.15-5.24(1H,m), 6.19-6.21(1H,m), 6.81(1H,d,J=9.6 Hz), 6.91(2H,s), 7.09(1H,d,J=9.6 Hz), 7.16-7.19(1H,m), 7.29-7.33(1H,m), 7.46-7.49(3H,m), 7.60-7.63(2H,m)

ESI/MS: 346 [M+H]⁺

### EXAMPLE 135

To a solution of 5-methoxy-3-(3-oxo-2-isopropyl-2,3-dihydropyridazin-6-yl)-2-phenylpyrazolo[1,5-a]pyridine (2.0 g) in AcOH (30 ml) was added pyridinium hydrobromide perbromide (2.67 g) and the mixture was stirred at ambient temperature for 1.5 hours. The reaction mixture was concentrated, diluted with AcOEt, washed with water, 5% sodium thiosulfate solution, 1N NaOH solution, and brine, dried over sodium sulfate, evaporated in vacuo. The residue was purified by silica gel column chromatography (CHCl₃-AcOEt, 3:1) to give 4-bromo-5-methoxy-3-(3-oxo-2-isopropyl-2,3-dihydropyridazin-6-yl)-2-phenylpyrazolo[1,5-a]pyridine (2.43 g) as a white solid.

mp: 205-206° C. (AcOEt)

IR (KBr): 2972, 1658, 1633, 1587, 1468, 1421 cm⁻¹

¹H NMR (CDCl₃, δ): 1.30(6H,d,J=6.6 Hz), 4.01(3H,s), 5.37(1H,h, J=6.6 Hz), 6.71(1H,d,J=7.6 Hz), 6.85(1H,d,J=9.5 Hz), 7.18(1H,d, J=9.5 Hz), 7.27-7.57(5H,m), 8.49(1H,d,J=7.6 Hz)

ESI/MS: 461, 463 [M+Na]⁺

### EXAMPLE 136

To a mixture of 3-(3-oxo-2-isopropyl-2,3-dihydropyridazin-6-yl)-2-phenylpyrazolo[1,5-a]pyridin-5-yl trifluoromethanesulfonate (60.0 mg), 2-methyl-3-butyn-2-ol (0.018 ml), dichlorobis (triphenylphosphine)palladium (II) (0.9 mg) and CuI (0.2 mg) in DMF (1 ml) was added triethylamine (0.053 ml) and the mixture was stirred at ambient temperature for 1 hour. The reaction mixture was diluted with AcOEt, washed with water and brine, dried over sodium sulfate, evaporated in vacuo. The residue was purified by silica gel column chromatography (n-hexane-AcOEt, 2:3) to give 5-(3-hydroxy-3-methyl-1-butynyl)-3-(3-oxo-2-isopropyl-2,3-dihydropyridazin-6-yl)-2-phenylpyrazolo[1,5-a]pyridine (50.7 mg) as a solid.

mp: 171-172° C. (AcOEt)

IR (KBr): 3273, 2979, 2222, 1647, 1574, 1527 cm⁻¹

¹H NMR (CDCl₃, δ): 1.47(6H,d,J=6.6 Hz), 1.65(6H,s), 5.43(1H,h, J=6.6 Hz), 6.77(1H,d,J=9.6 Hz), 6.85(1H,dd,J=7.1, 1.9 Hz), 7.01(1H, d,J=9.6 Hz), 7.37-7.67(5H,m), 7.98-8.03(1H,m), 8.42(1H,dd,J=7.1, 0.8 Hz)

ESI/MS: 435 [M+Na]⁺

### EXAMPLE 137

5-[(1-Hydroxycyclohexyl)ethynyl]-3-(3-oxo-2-isopropyl-2,3-dihydropyridazin-6-yl)-2-phenylpyrazolo[1,5-a]pyridine was prepared by similar procedure as that of Example 136.

IR (KBr): 3371, 2931, 2854, 2220, 1651, 1585, 1533 cm⁻¹

¹H NMR (CDCl₃, δ): 1.24-1.39(1H,m), 1.47(6H,d, J=6.6 Hz), 1.53-1.85(7H,m), 1.96-2.10(2H,m), 2.25(1H,s), 5.44(1H,h,J=6.6 Hz), 6.77(1H,d,J=9.6 Hz), 6.87(1H,dd,J=7.2, 1.8 Hz), 7.00(1H,d, J=9.6 Hz), 7.39-7.68(5H,m), 8.09(1H,dd,J=1.8, 0.8 Hz), 8.43(1H, dd,J=7.2, 0.8 Hz)

ESI/MS: 475 [M+Na]⁺

### EXAMPLE 138

5-Phenylethynyl-3-(3-oxo-2-isopropyl-2,3-dihydropyridazin-6-yl)-2-phenylpyrazolo[1,5-a]pyridine was prepared by similar procedure as that of Example 136.

mp: 185-186° C. (AcOEt)

IR (KBr): 29-74, 2214, 1653, 1628, 1583, 1527 cm⁻¹

¹H NMR (CDCl₃, δ): 1.50(6H,d,J=6.6 Hz), 5.44(1H,h,J=6.6 Hz), 6.78(1H,d,J=9.6 Hz), 6.95-7.00(1H,m), 7.03(1H,d,J=9.6 Hz), 7.35-7.50(6H,m), 7.50-7.65(4H,m), 8.15(1H,dd,J=1.8, 0.9 Hz), 8.47(1H,dd,J=7.1, 0.9 Hz)

ESI/MS: 453 [M+Na]⁺

### EXAMPLE 139

To a mixture of 3-(3-oxo-2-isopropyl-2,3-dihydropyridazin-6-yl)-2-phenylpyrazolo[1,5-a]pyridine-5-carboxylic acid (50 mg) and cyclopropylamine (0.012 ml) in DMF (2 ml) was added 1-hydroxybenzotriazole (27 mg) and 1-ethyl-3-(3′-dimethylaminopropyl)carbodiimide hydrochloride (51 mg). After stirring at ambient temperature for 24 hours, the reaction mixture was diluted with AcOEt, washed with 0.1N HCl, water, saturated sodium hydrogen carbonate solution, water and brine, dried over magnesium sulfate, and evaporated in vacuo. The residue was purified by silica gal column chromatography (CH₂Cl₂—MeOH, 20:1 elution) to give N-cyclopropyl-3-(3-oxo-2-isopropyl-2,3-dihydropyridazin-6-yl)-2-phenylpyrazolo[1,5-a]pyridine-5-carboxamide (53.6 mg) as an yellow solid.

mp: 184-185° C. (AcOEt)

IR (KBr): 3292, 3026, 2976, 1668, 1641, 1593, 1529 cm⁻¹

¹H NMR (CDCl₃, δ): 1.60-1.67(2H,m), 1.87-1.95(2H,m), 1.50(6H,d, J=6.6 Hz), 2.90-3.00(1H,m), 5.45(1H,h,J=6.6 Hz), 6.43(1H,s), 6.74(1H,d,J=9.6 Hz), 7.00(1H,d,J=9.6 Hz), 7.26(1H,dd,J=7.2, 2.0 Hz), 7.42-7.65(5H,m), 8.45(1H,dd, J=2.0, 0.8 Hz), 8.53(1H,dd, J=7.2, 0.8 Hz)

Negative ESI/MS: 412[M−H]⁻

### EXAMPLE 140

N-Cycloheptyl-3-(3-oxo-2-isopropyl-2,3-dihydropyridazin-6-yl)-2-phenylpyrazolo[1,5-a]pyridine-5-carboxamide was prepared by similar procedure as that of Example 139.

mp: 239-240° C. (AcOEt)

IR (KBr): 3309, 2925, 2858, 1666, 1630, 1593, 1527 cm⁻¹

¹H NMR (CDCl₃, δ): 1.45-1.74(10H,m), 1.52(6H,d,J=6.6 Hz), 2.01-2.23(2H,m), 4.12-4.24(1H,m), 5.47(1H,h,J=6.6 Hz), 6.14(1H,d, J=8.0 Hz), 6.76(1H,d,J=9.6 Hz), 7.01(1H,d,J=9.6 Hz), 7.31(1H,dd, J=7.2, 2.0 Hz), 7.43-7.65(5H,m), 8.43(1H,dd,J=2.0, 0.8 Hz), 8.54(1H,dd,J=7.2, 0.8 Hz)

Negative ESI/MS: 468[M−H]⁻

### EXAMPLE 141

5-[(Hexahydro-1H-azepin-1-yl)carbonyl]-3-(3-oxo-2-isopropyl-2,3-dihydropyridazin-6-yl)-2-phenylpyrazolo[1,5-a]pyridine was prepared by similar procedure as that of Example 139.

IR (KBr): 2927, 1664, 1630, 1591, 1533, 1479 cm⁻¹

¹H NMR (CDCl₃, δ): 1.45(6H,d,J=6.6 Hz), 1.55-1.78(6H,m), 1.78-1.92(2H,m), 3.43-3.53(2H,m), 3.65-3.75(2H,m), 5.42(1H,h, J=6.6 Hz), 6.77(1H,d,J=9.6 Hz), 6.93(1H,dd,J=7.1, 1.8 Hz), 7.01(1H, d,J=9.6 Hz), 7.41-7.65(5H,m), 7.99(1H,dd,J=1.8, 0.9 Hz), 8.54(1H, dd,J=7.1, 0.9 Hz)

ESI/MS: 478 [M+Na]⁺

### EXAMPLE 142

5-{[4-(2-Pyridinyl)-1-piperazinyl]carbonyl}-3-(3-oxo-2-isopropyl-2,3-dihydropyridazin-6-yl)-2-phenylpyrazolo[1,5-a]pyridine was prepared by similar procedure as that of Example 139.

IR (KBr): 2978, 1662, 1635, 1591, 1533, 1477, 1433 cm⁻¹

¹HNMR (CDCl₃, δ): 1.44(6H,d,J=6.6 Hz), 3.50-4.00(8H,m), 5.41(1H, h,J=6.6 Hz), 6.65-6.73(2H,m), 6.77(1H,d,J=9.6 Hz), 6.96-7.00(1H, m), 7.01(1H,d,J=9.6 Hz), 7.42-7.65(6H,m), 8.09(1H,dd,J=1.8, 0.9 Hz), 8.17-8.23(1H,m), 8.58(1H,dd,J=7.1, 0.9 Hz)

ESI/MS: 542 [M+Na]⁺

### EXAMPLE 143

N-(n-Butyl)-3-(3-oxo-2-isopropyl-2,3-dihydropyridazin-6-yl)-2-phenylpyrazolo[1,5-a]pyridine-5-carboxamide was prepared by similar procedure as that of Example 139.

mp: 185-186° C. (AcOEt)

IR (KBr): 3303, 2958, 2931, 2871, 1668, 1641, 1593, 1533 cm⁻¹

¹HNMR (CDCl₃, δ): 0.98(3H,t,J=7.3 Hz), 1.38-1.50(2H,m), 1.51(6H, d,J=6.6 Hz), 1.55-1.67(2H,m), 3.45-3.55(2H,m), 5.45(1H,h), 6.15-6.25(1H,m), 6.75(1H,d,J=9.6 Hz), 7.01(1H,d,J=9.6 Hz), 7.29(1H,dd,J=7.2, 2.0 Hz), 7.42-7.65(5H,m), 8.40-8.46(1H,m), 8.55(1H,dd,J=7.2, 0.8 Hz)

Negative ESI/MS: 428[M−H]⁻

### EXAMPLE 144

N-(2-Pyridinylmethyl)-3-(3-oxo-2-isopropyl-2,3-dihydropyridazin-6-yl)-2-phenylpyrazolo[1,5-a]pyridine-5-carboxamide was prepared by similar procedure as that of Example 139.

mp: 193-194° C. (AcOEt)

IR (KBr): 3273, 3051, 2979, 2933, 1666, 1637, 1595, 1533 cm⁻¹

¹H NMR (CDCl₃, δ): 1.50(6H,d,J=6.6 Hz), 4.80(2H,d,J=4.4 Hz), 5.44(1H,h,J=6.6 Hz), 6.77(1H,d,J=9.6 Hz), 7.03(1H,d,J=9.6 Hz), 7.22-7.28(1H,m), 7.28-7.35(1H,m), 7.38-7.45(1H,m), 7.45-7.66(5H,m), 7.68-7.77(1H,m), 7.87-7.95(1H,m), 8.55-8.62(3H,m)

Negative ESI/MS: 463[M−H]⁻

### EXAMPLE 145

A mixture of 3-(3-oxo-2-isopropyl-2,3-dihydropyridazin-6-yl)-2-phenylpyrazolo[1,5-a]pyridine-5-carboxylic acid (8.38 g), Et3N (4.06 ml), and diphenylphosphoryl azide (6.28 ml) in t-BuOH-toluene (2:1, 210 ml) was stirred at 80° C. for 22 hours. After cooling to ambient temperature, the mixture was evaporated, diluted with AcOEt, washed with saturated sodium hydrogen carbonate solution, water and brine, dried over sodium sulfate, and evaporated in vacuo. The residue was purified by silica gal column chromatography (CHCl₃-EtOAc (2:1) to CH₂Cl₂—MeOH (10:1) to give tert-butyl 3-(3-oxo-2-isopropyl-2,3-dihydropyridazin-6-yl)-2-phenylpyrazolo[1,5-a]pyridin-5-ylcarbamate (10.76 g) as a pale yellow solid and N,N′-bis[3-(3-oxo-2-isopropyl-2,3-dihydropyridazin-6-yl)-2-phenylpyrazolo[1,5-a]pyridin-5-yl]urea (902 mg) as an yellow solid.

Tert-Butyl 3-(3-oxo-2-isopropyl-2,3-dihydropyridazin-6-yl)-2-phenylpyrazolo[1,5-a]pyridin-5-ylcarbamate

mp: 208-210° C. (AcOEt)

IR (KBr): 1728, 1645, 1579, 1514 cm⁻¹

¹H NMR (CDCl₃, δ): 1.50(6H,d,J=6.7 Hz), 1.54(9H,s), 5.42(1H,h, J=6.7 Hz), 6.73(1H,d,J=9.6 Hz), 6.79 (1H,dd,J=7.5, 2.4 Hz), 7.48 (1H,d,J=9.6 Hz), 7.37-7.50(3H,m), 7.52-7.65(2H,m), 8.25-8.30(1H, m), 8.38(1H,dd,J=7.5, 0.6 Hz)

ESI/MS: 468 [M+Na]⁺

N,N′-Bis[3-(3-oxo-2-isopropyl-2,3-dihydropyridazin-6-yl)-2-phenylpyrazolo[1,5-a]pyridin-5-yl]urea

ESI/MS: 739 [M+Na]⁺

### EXAMPLE 146

A solution of tert-butyl 3-(2-isopropyl-3-oxo-2,3-dihydropyridazin-6-yl)-2-phenylpyrazolo[1,5-a]pyridin-5-ylcarbamate (9.58 g) in TFA (60 ml) was stirred at ambient temperature for 2 hours. After evaporation, the residue was dissolved in AcOEt, and 3N NaOH solution was added thereto under ice-cooling. The mixture was extracted with AcOEt, washed with water and brine, dried over sodium sulfate, and evaporated in vacuo. The residue was purified by silica gal column chromatography (CHCl₃-MeOH, 10:1) to give 5-amino-3-(3-oxo-2-isopropyl-2,3-dihydropyridazin-6-yl)-2-phenylpyrazolo[1,5-a]pyridine (4.90 g) as an yellow solid.

mp: 196-197° C. (AcOEt)

IR (KBr): 3425, 3325, 3222, 1651, 1583, 1537, 1469 cm⁻¹

¹H NMR (CDCl₃, δ): 1.47(6H,d,J=6.6 Hz), 4.08(2H,s), 5.42(1H,h, J=6.6 Hz), 6.37(1H,dd,J=7.4, 2.6 Hz), 6.70(1H,d,J=9.6 Hz), 6.95(1H, d,J=9.6 Hz), 7.10(1H,d,J=2.6 Hz), 7.38-7.63(5H,m), 8.28(1H,d, J=7.4 Hz)

ESI/MS: 368 [M+Na]⁺

### EXAMPLE 147

To a solution of 5-amino-3-(3-oxo-2-isopropyl-2,3-dihydropyridazin-6-yl)-2-phenylpyrazolo[1,5-a]pyridine (60 mg) and pyridine (0.042 ml) in CH₂Cl₂ (1.5 ml) was added AcCl (0.015 ml) at 5° C. and the mixture was stirred at the same temperature for 10 minutes. The reaction mixture was diluted with AcOEt, washed with 1N HCl, water, saturated sodium hydrogen carbonate solution, water and brine, dried over sodium sulfate, and evaporated in vacuo. The residue was purified by silica gal column chromatography (CHCl₃-MeOH, 10:1) to give 5-acetylamino-3-(3-oxo-2-isopropyl-2,3-dihydropyridazin-6-yl)-2-phenylpyrazolo[1,5-a]pyridine (68.6 mg) as a solid.

mp: 221-222° C. (AcOEt-n-Hexane)

IR (KBr): 3294, 3249, 3128, 3043, 2981, 1702, 1649, 1576, 1450 cm⁻¹

¹H NMR (CDCl₃, δ): 1.50(6H,d,J=6.6 Hz), 2.25(3H,s), 5.42(1H,h, J=6.6 Hz), 6.73(1H,d,J=9.6 Hz), 6.89(1H,dd,J=7.4, 2.4 Hz), 6.98(1H, d,J=9.6 Hz), 7.40-7.63(5H,m), 7.83(1H,s), 8.41(1H,d,J=7.4 Hz), 8.49(1H,s)

Negative ESI/MS: 386[M−H]⁻

### EXAMPLE 148

5-Benzoylamino-3-(3-oxo-2-isopropyl-2,3-dihydropyridazin-6-yl)-2-phenylpyrazolo[1,5-a]pyridine was prepared by similar procedure as that of Example 147.

mp: 266-267° C. (AcOEt)

IR (KBr): 3318, 2978, 1678, 1647, 1572, 1508, 1491 cm⁻¹

¹H NMR (CDCl₃, δ): 1.38(6H,d,J=6.6 Hz), 5.24(1H,h,J=6.6 Hz), 6.86(1H,d,J=9.6 Hz), 7.08(1H,d,J=9.6 Hz), 7.29(1H,dd,J=7.5, 2.3 Hz), 7.43-7.67(8H,m), 7.93-8.00(2H,m), 8.75-8.83(2H,m), 10.63(1H,s)

Negative ESI/MS: 448[M−H]⁻

### EXAMPLE 149

5-(2-Methylpropanoylamino)-3-(3-oxo-2-isopropyl-2,3-dihydropyridazin-6-yl)-2-phenylpyrazolo[1,5-a]pyridine was prepared by similar procedure as that of Example 147.

mp: 199-200° C. (AcOEt)

IR (KBr): 3315, 2966, 1701, 1645, 1574, 1510, 1489 cm⁻¹

¹H NMR (CDCl₃, δ): 1.29(6H,d,J=6.9 Hz), 1.47(6H,d,J=6.6 Hz), 2.58(1H,h,J=6.9 Hz), 5.40(1H,h,J=6.6 Hz), 6.75(1H,d,J=9.6 Hz), 6.95(1H,dd,J=7.4, 2.5 Hz), 7.01(1H,d,J=9.6 Hz), 7.39-7.47(3H,m), 7.52(1H,s), 7.55-7.64(2H,m), 8.37-8.44(2H,m)

Negative ESI/MS: 414[M−H]⁻

### EXAMPLE 150

5-Methylsulfonylamino-3-(3-oxo-2-isopropyl-2,3-dihydropyridazin-6-yl)-2-phenylpyrazolo[1,5-a]pyridine was prepared by similar procedure as that of Example 147.

mp: 193-194° C. (AcOEt)

IR (KBr): 3078, 2873, 1643, 1576, 1485, 1419, 1338 cm⁻¹

¹H NMR (CDCl₃, δ): 1.50(6H,d,J=6.6 Hz), 3.15(3H,s), 5.43(1H,h, J=6.6 Hz), 6.70-6.79(2H,m), 6.97(1H,d,J=9.6 Hz), 7.40-7.67(6H,m), 7.94(1H,dd,J=2.5, 0.7 Hz), 8.46(1H,dd,J=7.4, 0.7 Hz)

Negative ESI/MS: 422[M−H]⁻

### EXAMPLE 151

Methyl 3-(3-oxo-2-isopropyl-2,3-dihydropyridazin-6-yl)-2-phenylpyrazolo[1,5-a]pyridin-5-ylcarbamate was prepared by similar procedure as that of Example 147.

mp: 213-214° C. (AcOEt)

IR (KBr): 3275, 1736, 1649, 1583, 1520 cm⁻¹

¹H NMR (CDCl₃, δ): 1.49(6H,d,J=6.6 Hz), 3.83(3H,s), 5.43(1H,h, J=6.6 Hz), 6.74(1H,d,J=9.6 Hz), 6.88(1H,dd,J=7.5, 2.4 Hz), 6.98(1H, d, J=9.6 Hz), 7.014(1H,s), 7.38-7.65(5H,m), 8.15-8.23(1H,m), 8.41(1H,d,J=7.5 Hz)

Negative ESI/MS: 402[M−H]⁻

### EXAMPLE 152

5-(4-Bromobutanoylamino)-3-(3-oxo-2,3-2-isopropyl-dihydropyridazin-6-yl)-2-phenylpyrazolo[1,5-a]pyridine was prepared by similar procedure as that of Example 147.

IR (KBr): 3292, 3249, 1701, 1647, 1579, 1506 cm⁻¹

¹H NMR (CDCl₃, δ): 1.48(6H,d,J=6.6 Hz), 2.25-2.35(2H,m), 2.64(2H, t,J=7.0 Hz), 3.55(2H,t,J=6.2 Hz), 5.41(1H,h,J=6.6 Hz), 6.75(1H,d, J=9.6 Hz), 6.95(1H,dd,J=7.6, 2.2 Hz), 6.99(1H,d,J=9.6 Hz), 7.38-7.63(5H,m), 7.82(1H,s), 8.37-8.45(2H,m)

### EXAMPLE 153

To a solution of 5-(4-bromobutanoylamino)-3-(3-oxo-2-isopropyl-2,3-dihydropyridazin-6-yl)-2-phenylpyrazolo [1,5-a]pyridine (90 mg) in DMF (3 ml) was added NaH (60% oil suspension, 8.0 mg) at 5° C. and the mixture was stirred at ambient temperature for 3 hours. The reaction mixture was poured into water, and the resulting precipitate was collected by filtration, washed with ether to give 5-(2-oxo-1-pyrrolidinyl)-3-(3-oxo-2-isopropyl-2,3-dihydropyridazin-6-yl)-2-phenylpyrazolo [1,5-a]pyridine (64.0 mg).

mp: 266-267° C. (AcOEt)

IR (KBr): 2978, 1697, 1657, 1641, 1587, 1531 cm⁻¹

¹H NMR (CDCl₃, δ): 1.51(6H,d,J=6.6 Hz), 2.18-2.30(2H,m), 2.65-2.73(2H,m), 3.87-3.95(2H,m), 5.46(1H,h,J=6.6 Hz), 6.73(1H,d, J=9.6 Hz), 6.98(1H,d,J=9.6 Hz), 7.41-7.63(5H,m), 7.86(1H,dd, J=7.6, 2.5 Hz), 7.87-7.92(1H,m), 7.46(1H,dd,J=7.6, 0.5 Hz)

ESI/MS: 436 [M+Na]⁺

### EXAMPLE 154

To a solution of 5-amino-3-(3-oxo-2-isopropyl-2,3-dihydropyridazin-6-yl)-2-phenylpyrazolo[1,5-a]pyridine (60 mg), 35% formaldehyde solution (0.6 ml) and AcOH (0.020 ml) in CH₂Cl₂-MeOH (4:1, 7.5 ml) was added sodium triacetoxyborohydride (184 mg). After stirring at ambient temperature for 15 hours, the reaction mixture was diluted with AcOEt, washed with saturated sodium hydrogen carbonate solution, water, and brine, dried over sodium sulfate, evaporated in vacuo. The residue was purified by silica gel column chromatography (n-hexane-AcOEt, 2:5) to give 5-[bis(methoxymethyl)amino]-3-(3-oxo-2-isopropyl-2,3-dihydropyridazin-6-yl)-2-phenylpyrazolo[1,5-a]pyridine (29.7 mg) as a green solid.

mp: 148-150° C. (AcOEt)

IR (KBr): 2929, 1651, 1585, 1522 cm⁻¹

¹H NMR (CDCl₃, δ): 1.50(6H,d,J=6.6 Hz), 3.35(6H,s), 4.88(4H,s), 5.43(1H,h,J=6.6 Hz), 6.70(1H,d,J=9.6 Hz), 6.82(1H,dd,J=7.7, 2.6 Hz), 6.94(1H,d, J=9.6 Hz), 7.38-7.64(6H,m), 8.36(1H,d, J=7.7 Hz)

ESI/MS: 456 [M+Na]⁺

### EXAMPLE 155

To a mixture of 5-(4-piperidinyloxy)-3-(3-oxo-2-isopropyl-2,3-dihydropyridazin-6-yl)-2-phenylpyrazolo[1,5-a]pyridine hydrochloride (150 mg), 35% aq.HCHO (0.56 ml), and AcOH (0.018 ml) in CH₂Cl₂ (3.6 ml)-MeOH (0.9 ml) was added NaBH(OAc)₃(136 mg), and the mixture was stirred at ambient temperature for 1 hour. To the reaction mixture was added saturated sodium hydrogen carbonate solution (20 ml) and water (20 ml), and the mixture was extracted with AcOEt (40 ml×2). The organic layer was washed with water (30 ml) and brine, dried over magnesium sulfate, filtered, and evaporated in vacuo. The residue was purified by silica-gel (6 g) column chromatography (CHCl₃:MeOH=9:1). The solid (40 mg) was dissolved in 1,4-dioxane (1 ml), and to the solution was added 4N—HCl in dioxane, then the resultant precipitate was collected by filteration and washed with Et₂O and IPE, and dried to give 5-[(1-methyl-4-piperidinyl)oxy]-3-(3-oxo-2-isopropyl-2,3-dihydropyridazin-6-yl)-2-phenylpyrazolo[1,5-a]pyridine hydrochloride (31.0 mg).

mp: >250° C. (AcOEt)

NMR (DMSO-d6, δ): 1.32(6H, d, J=6.6 Hz), 1.80-2.40(4H,m), 2.78(3H, s), 2.90-3.70(4H, m), 4.55-4.95(1H, m), 5.05-5.35(1H,m), 6.70-6.95(2H, m), 7.10(1H, d, J=9.6 Hz), 7.26(1H, d, J=2.4 Hz), 7.40-7.65(5H, m), 8.73(1H, d, J=6.9 Hz), 10.31(1H, br,s)

ESI/MS: 444[M−HCl+H]⁺

### EXAMPLE 156

1,1-Dibenzyl-4-{[3-(3-oxo-2-isopropyl-2,3-dihydropyridazin-6-yl)-2-phenylpyrazolo[1,5-a]pyridine]oxy}piperidinium bromide was prepared by similar procedure as that of Example 55.

mp: 211-213.5° C. (AcOEt)

NMR (DMSO-d6, δ): 1.23(6H, d, J=6.6 Hz), 2.25-2.65(4H, m), 3.15-3.60(4H, m), 4.60-4.90(4H, m), 5.05-5.35(1H, m), 6.43(1H, dd, J=2.2, 7.5 Hz), 6.84(1H, d, J=9.6 Hz), 7.04(1H, d, J=9.6 Hz), 7.14(1H, d, J=2.4 Hz), 7.35-7.75(15H, m), 8.68(1H, d, J=7.5 Hz)

ESI/MS: 610[M+Na]⁺

### EXAMPLE 157

5-[(4-Methoxybenzyl)oxy]-3-(3-oxo-2-isopropyl-2,3-dihydropyridazin-6-yl)-2-phenylpyrazolo[1,5-a]pyridine was prepared by similar procedure as that of Example 8.

NMR (DMSO-d6, δ): 1.30(6H, d, J=6.6 Hz), 3.77(3H, s), 5.13(2H, s), 5.15-5.30(1H, m), 6.80(1H, dd, J=2.7, 7.5 Hz), 6.85(1H, d, J=9.6 Hz), 6.98(2H, td, J=2.4, 9.1 Hz), 7.08(1H, d, J=9.6 Hz), 7.34(1H, d, J=2.6 Hz), 7.41(2H, td, J=2.4, 9.2 Hz), 7.44-7.60(5H, m), 8.70(1H, d, J=7.5 Hz)

ESI/MS: 489[M+Na]⁺

### EXAMPLE 158

5-{[(4-Trifluoromethyl)-2-pyridinyl]oxy}-3-(3-oxo-2-isopropyl-2,3-dihydropyridazin-6-yl)-2-phenylpyrazolo[1,5-a]pyridine was prepared by similar procedure as that of Example 8.

NMR (DMSO-d6, δ): 1.16(6H, d, J=6.6 Hz), 5.10-5.25(1H,m), 6.82(1H, d, J=9.7 Hz), 7.05-7.10(2H, m), 7.45-7.55(4H, m), 7.55-7.63(2H, m), 7.65(1H, d, J=2.1 Hz), 8.35(1H, dd, J=2.5, 8.7 Hz), 8.68(1H, s), 8.91(1H, d, J=7.5 Hz)

ESI/MS: 514[M+Na]⁺

### EXAMPLE 159

5-(Nicotinamid-6-oxy)-3-(3-oxo-2-isopropyl-2,3-dihydropyridazin-6-yl)-2-phenylpyrazolo[1,5-a]pyridine was prepared by similar procedure as that of Example 31.

NMR (DMSO-D6, δ): 1.15(6H, d, J=6.6 Hz), 5.05-5.20(1H, m), 6.81(1H, d, J=9.6 Hz), 7.00-7.10(2H, m), 7.33(1H, d, J=8.6 Hz), 7.45-7.55(3H, m), 7.55-7.65(4H, m), 8.13(1H, s), 8.38(1H, dd,J=2.5, 8.5 Hz), 8.74(1H, d, J=2.3 Hz), 8.89(1H, d, J=7.5 Hz)

ESI/MS: 489[M+Na]⁺

### EXAMPLE 160

5-[2-Oxo-2-(1-piperidinyl)ethoxy]-3-(3-oxo-2-isopropyl-2,3-dihydropyridazin-6-yl)-2-phenylpyrazolo[1,5-a]pyridine was prepared by similar procedure as that of Example 50.

NMR (DMSO-d6, δ): 1.33(6H, d, J=6.6 Hz), 1.40-1.50(2H, m), 1.50-1.65(4H, m), 3.30-3.40(2H, m), 3.40-3.50(2H,m), 4.98(2H, s), 5.10-5.30(1H, m), 6.75-6.90(2H, m), 7.02(1H, d, J=9.6 Hz), 7.16(1H, d, J=2.7 Hz), 7.40-7.50(3H, m), 7.50-7.60(2H, m), 8.68(1H, d, J=7.5 Hz)

ESI/MS: 494[M+Na]⁺

### EXAMPLE 161

N-(tert-Butyl)-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 50.

NMR (DMSO-d6, δ): 1.20-1.45(15H, m), 4.54(2H, s), 5.15-5.30(1H, m), 6.80-6.90(2H,m), 7.10(1H, d, J=9.6 Hz), 7.14(1H, d, J=2.7 Hz), 7.40-7.55(3H, m), 7.55-7.60(2H, m), 7.62(1H, s), 8.71(1H, d, J=7.5 Hz)

Negative ESI/MS: 458[M−H]⁻

### EXAMPLE 162

N-Cycloheptyl-2-{[3-(3-oxo-2-isopropyl-2,3-dihydropyridazin-6-yl)-2-phenylpyrazolo[1,5-a]pyridin-5-yl]oxy}acetamide was prepared by similar procedure as that of Example 50.

NMR (DMSO-d6, δ): 1.29(6H, d, J=6.6 Hz), 1.20-1.65(10H, m), 1.65-1.85(2H, m), 3.70-3.90(1H, m), 4.58(2H, s), 5.10-5.28(1H, m), 6.78-6.90(2H, m), 7.10(1H, d, J=9.6 Hz), 7.14(1H, d, J=2.6 Hz), 7.40-7.52(3H, m), 7.52-7.60(2H, m), 8.02(1H, d, J=8.0 Hz), 8.72(1H, d, J=7.6 Hz)

Negative ESI/MS: 498[M−H]⁻

### EXAMPLE 163

3-[2,3-Dihydropyridazin-6-yl)-6-methoxy-2-phenylpyrazolo[1,5-a]pyridine was prepared by similar procedure as that of Example 5.

NMR (DMSO-d6, δ): 3.82(3H,s), 6.75(1H, d, J=3.9 Hz), 6.90-7.00(2H, m), 7.25-7.60(6H, m), 8.41(1H, d, J=3.4 Hz), 12.97(1H,s)

Negative ESI/MS: 317[M−H]⁻

### EXAMPLE 164

6-Methoxy-3-(3-oxo-2-isopropyl-2,3-dihydropyridazin-6-yl)-2-phenylpyrazolo[1,5-a]pyridine was prepared by similar procedure as that of Example 2.

ESI/MS: 383[M+Na]⁺

### EXAMPLE 165

6-Hydroxy-3-(3-oxo-2-isopropyl-2,3-dihydropyridazin-6-yl)-2-phenylpyrazolo[1,5-a]pyridine was prepared by similar procedure as that of Example 3.

ESI/MS: 369[M+Na]⁺

### EXAMPLE 166

6-[2-(N,N-Dimethylamino)ethoxy]-3-(3-oxo-2-isopropyl-2,3-dihydropyridazin-6-yl)-2-phenylpyrazolo[1,5-a]pyridine was prepared by similar procedure as that of Example 8.

### EXAMPLE 167

6-{2-[(2R,6S)-2,6-Dimethyl-4-morpholinyl]ethoxy}-3-(3-oxo-2-isopropyl-2,3-dihydropyridazin-6-yl)-2-phenylpyrazolo[1,5-a]pyridine was prepared by similar procedure as that of Example 8.

ESI/MS: 488[M+H]⁺

### EXAMPLE 168

6-(2-Pyridinylmethoxy)-3-(3-oxo-2-isopropyl-2,3-dihydropyridazin-6-yl)-2-phenylpyrazolo[1,5-a]pyridine was prepared by similar procedure as that of Example 8.

ESI/MS: 460[M+Na]⁺

### EXAMPLE 169

2-{[3-(3-Oxo-2-isopropyl-2,3-dihydropyridazin-6-yl)-2-phenylpyrazolo[1,5-a]pyridin-6-yl]oxy}acetic acid was prepared by similar procedure as that of Example 8.

ESI/MS: 441[M+Na]⁺

### EXAMPLE 170

Ethyl 2-{[3-(3-oxo-2-isopropyl-2,3-dihydropyridazin-6-yl)-2-phenylpyrazolo[1,5-a]pyridin-6-yl]oxy}acetic acid was prepared by similar procedure as that of Example 39.

Negative ESI/MS: 403[M−H]⁻

### EXAMPLE 171

N,N-Dimethyl-2-{[3-(3-oxo-2-isopropyl-2,3-dihydropyridazin-6-yl)-2-phenylpyrazolo[1,5-a]pyridin-6-yl]oxy}acetamide was prepared by similar procedure as that of Example 50.

ESI/MS: 454[M+Na]⁺

### EXAMPLE 172

6-[2-(1-Pyrrolidinyl)-2-oxo-ethoxy]-3-(3-oxo-2-isopropyl-2,3-dihydropyridazin-6-yl)-2-phenylpyrazolo[1,5-a]pyridine was prepared by similar procedure as that of Example 50.

ESI/MS: 480[M+Na]⁺

### EXAMPLE 173

6-[2-(4-Methyl-1-piperazinyl)-2-oxo-ethoxy]-3-(3-oxo-2-isopropyl-2,3-dihydropyridazin-6-yl)-2-phenylpyrazolo[1,5-a]pyridine was prepared by similar procedure as that of Example 50.

ESI/MS: 487[M+H]⁺

### EXAMPLE 174

6-{2-[(2R,6S)-2,6-Dimethyl-4-morpholinyl]-2-oxo-ethoxy}-3-(3-oxo-2-isopropyl-2,3-dihydropyridazin-6-yl)-2-phenylpyrazolo[1,5-a]pyridine was prepared by similar procedure as that of Example 50.

ESI/MS: 524[M+Na]⁺

### EXAMPLE 175

N-Cyclopentyl-2-{[3-(3-oxo-2-isopropyl-2,3-dihydropyridazin-6-yl)-2-phenylpyrazolo[1,5-a]pyridin-6-yl]oxy}acetamide was prepared by similar procedure as that of Example 50.

ESI/MS: 494[M+Na]⁺

### EXAMPLE 176

N-Isopropyl-2-{[3-(3-oxo-2-isopropyl-2,3-dihydropyridazin-6-yl)-2-phenylpyrazolo [1,5-a]pyridin-6-yl]oxy}acetamide was prepared by similar procedure as that of Example 50.

ESI/MS: 468[M+Na]⁺

### EXAMPLE 177

N-Isobutyl-2-{[3-(3-oxo-2-isopropyl-2,3-dihydropyridazin-6-yl)-2-phenylpyrazolo[1,5-a]pyridin-6-yl]oxy}acetamide was prepared by similar procedure as that of Example 50.

ESI/MS: 482[M+Na]⁺

### EXAMPLE 178

6-Trifluoromethanesulfonoxy-3-(3-oxo-2-isopropyl-2,3-dihydropyridazin-6-yl)-2-phenylpyrazolo[1,5-a]pyridine was prepared by similar procedure as that of Example 68.

ESI/MS: 501[M+Na]⁺

### EXAMPLE 179

6-[(2R,6S)-2,6-Dimethyl-4-morpholinyl]-3-(3-oxo-2-isopropyl-2,3-dihydropyridazin-6-yl)-2-phenylpyrazolo[1,5-a]pyridine was prepared by similar procedure as that of Example 96.

ESI/MS: 466[M+Na]⁺

### EXAMPLE 180

6-(4-Methyl-1-piperazinyl)-3-(3-oxo-2-isopropyl-2,3-dihydropyridazin-6-yl)-2-phenylpyrazolo[1,5-a]pyridine was prepared by similar procedure as that of Example 96.

ESI/MS: 451[M+Na]⁺

### EXAMPLE 181

Methyl 3-(3-oxo-2-isopropyl-2,3-dihydropyridazin-6-yl)-2-phenylpyrazolo[1,5-a]pyridine-6-carboxylate was prepared by similar procedure as that of Example 69.

ESI/MS: 411[M+Na]⁺

### EXAMPLE 182

3-(3-Oxo-2-isopropyl-2,3-dihydropyridazin-6-yl)-2-phenylpyrazolo[1,5-a]pyridine-6-carboxylic acid was prepared by similar procedure as that of Example 70.

ESI/MS: 397[M+Na]⁺

### EXAMPLE 183

N,N-Dimethyl-[3-(3-oxo-2-isopropyl-2,3-dihydropyridazin-6-yl)-2-phenylpyrazolo[1,5-a]pyridine]-6-carboxamide was prepared by similar procedure as that of Example 50.

ESI/MS: 424[M+Na]⁺

### EXAMPLE 184

6-[(4-Methyl-1-piperazinyl)carbonyl]-3-(3-oxo-2-isopropyl-2,3-dihydropyridazin-6-yl)-2-phenylpyrazolo[1,5-a]pyridine was prepared by similar procedure as that of Example 50.

ESI/MS: 479[M+Na]⁺

### EXAMPLE 185

6-{[(2R,6S)-2,6-Dimethyl-4-morpholinyl]carbonyl}-3-(3-oxo-2-isopropyl-2,3-dihydropyridazin-6-yl)-2-phenylpyrazolo[1,5-a]pyridine was prepared by similar procedure as that of Example 50.

ESI/MS: 494[M+Na]⁺

### EXAMPLE 186

N-Isobutyl-[3-(3-oxo-2-isopropyl-2,3-dihydropyridazin-6-yl)-2-phenylpyrazolo[1,5-a]pyridine]-6-carboxamide was prepared by similar procedure as that of Example 50.

ESI/MS: 452[M+Na]⁺

### EXAMPLE 187

7-Acetylamino-3-(3-oxo-2-isopropyl-2,3-dihydropyridazin-6-yl)-2-phenylpyrazolo[1,5-a]pyridine was prepared by similar procedure as that of Example 147.

mp: 92-96° C.

NMR (DMSO-d6, δ): 1.30(6H,d,J=6.6 Hz), 2.33(3H,s), 5.14-5.28(1H,m), 6.86(1H,d,J=9.7 Hz), 7.14(1H,d,J=9.7 Hz), 7.38-7.69(8H,m), 10.50(1H,s)

ESI/MS: 388[M+H]⁺

### EXAMPLE 188

7-Methoxy-3-(3-oxo-2,3-dihydropyridazin-6-yl)-2-phenylpyrazolo[1,5-a]pyridine was prepared by similar procedure as that of Example 1.

NMR (CDCl₃, δ): 4.17(3H,s), 6.22(1H,d,J=7.3 Hz), 6.82(1H,d, J=9.8 Hz), 7.07(1H,d,J=9.8 Hz), 7.24(1H,t,J=8.3 Hz), 7.35-7.55(3H, m), 7.55-7.70(2H,m), 7.71(1H,d,J=8.7 Hz), 13.08(1H,s)

APCI/MS: 319[M+H]⁺

### EXAMPLE 189

7-Methoxy-3-(3-oxo-2-isopropyl-2,3-dihydropyridazin-6-yl)-2-phenylpyrazolo[1,5-a]pyridine was prepared by similar procedure as that of Example 2.

NMR(DMSO-d6, δ):1.30(6H,d,J=6.6 Hz), 4.16(3H,s), 5.10-5.35(1H, m), 6.57(1H,dd,J=7.1, 1.5 Hz), 6.85(1H,d,J=9.6 Hz), 7.12(1H,d, J=9.6 Hz), 7.35-7.65(7H,m)

APCI/MS: 361[M+H]⁺

### EXAMPLE 190

7-Methoxy-3-(3-oxo-2-methyl-2,3-dihydropyridazin-6-yl)-2-phenylpyrazolo[1,5-a]pyridine was prepared by similar procedure as that of Example 2.

NMR (DMSO-d6, δ): 3.75(3H,s), 4.15(3H,s), 6.57(1H,d,J=6.7 Hz), 6.85(1H,d,J=9.6 Hz), 7.04(1H,d,J=9.6 Hz), 7.35-7.55(4H,m), 7.55-7.70(3H,m)

APCI/MS: 333[M+H]⁺

### EXAMPLE 191

7-Methoxy-3-(3-oxo-2-ethyl-2,3-dihydropyridazin-6-yl)-2-phenylpyrazolo[1,5-a]pyridine was prepared by similar procedure as that of Example 2.

NMR(DMSO-d6, δ): 1.36(3H,t,J=7.2 Hz), 4.00-4.30(5H,m), 6.58(1H, dd,J=7.4, 1.0 Hz), 6.86(1H,d,J=9.6 Hz), 7.09(1H,d,J=9.6 Hz), 7.30-7.75(7H,m)

APCI/MS: 347[M+H]⁺

### EXAMPLE 192

7-Methoxy-3-(3-oxo-2-n-propyl-2,3-dihydropyridazin-6-yl)-2-phenylpyrazolo[1,5-a]pyridine was prepared by similar procedure as that of Example 2.

NMR (DMSO-d6, δ): 0.92(3H,t,J=7.4 Hz), 1.78(2H, 6-plet, J=7.3 Hz), 4.00-4.20(5H,m), 6.57(1H,dd,J=7.3, 1.3 Hz), 6.86(1H,d, J=9.6 Hz), 7.09(1H,d,J=9.6 Hz), 7.30-7.75(7H,m)

APCI/MS: 361[M+H]⁺

### EXAMPLE 193

4-Methoxy-3-(3-oxo-2,3-dihydropyridazin-6-yl)-2-phenylpyrazolo[1,5-a]pyridine was prepared by similar procedure as that of Example 1.

NMR(DMSO-d6, δ): 3.87(3H,s), 6.75(1H,d, J=7.6 Hz), 6.80-7.00(2H, m), 7.23-7.65(6H,m), 8.41(1H,d,J=6.7 Hz), 12.97(1H,s)

APCI/MS: 319[M+H]⁺

### EXAMPLE 194

4-Methoxy-3-(3-oxo-2-isopropyl-2,3-dihydropyridazin-6-yl)-2-phenylpyrazolo[1,5-a]pyridine was prepared by similar procedure as that of Example 2.

mp: 185-186° C. (AcOEt)

NMR(DMSO-d6, δ):1.01(6H,d,J=6.6 Hz), 3.84(3H,s), 4.95-5.20(1H, m), 6.77(1H,d,J=7.7 Hz), 6.90(1H,d,J=9.6 Hz), 6.95(1H,t,J=7.3 Hz), 7.30-7.50(5H,m), 7.53(1H,d,J=9.6 Hz) 8.42 (1H,d,J=6.8 Hz)

APCI/MS: 361[M+H]⁺

Anal.Calcd for C₂₁H₂₀N₄O₂: C, 69.98; H, 5.59; N, 15.55 Found: C, 70.19; H, 5.68; N, 15.54

### EXAMPLE 195

4-Hydroxy-3-(3-oxo-2-isopropyl-2,3-dihydropyridazin-6-yl)-2-phenylpyrazolo[1,5-a]pyridine was prepared by similar procedure as that of Example 3.

mp: 229-230° C. (EtOH)

NMR (DMSO-d6, δ): 1.01(6H,d,J=6.6 Hz), 4.93-5.20(1H,m), 6.59(1H, d,J=7.5 Hz), 6.82(1H,d,J=7.0 Hz), 6.90(1H,d,J=9.4 Hz), 7.26-7.65(6H,m), 8.31(1H,d,J=6.8 Hz), 10.73(1H,s)

APCI/MS: 347[M+H]⁺

Anal.Calcd for C₂₀H₁₈N₄O₂: C, 69.35; H, 5.24; N, 16.17 Found: C, 69.73; H, 5.23; N, 16.23

### EXAMPLE 196

To a mixture of 4-hydroxy-3-(3-oxo-2-isopropyl-2,3-dihydropyridazin-6-yl)-2-phenylpyrazolo[1,5-a]pyridine (80.0 mg) and potassium carbonate (96.0 mg) in DMF (3 ml) was added ethyl iodide (0.022 ml) and stirred at 60° C. for 1 hour. The reaction mixture was diluted with AcOEt, washed with water and brine, dried over sodium sulfate, evaporated in vacuo. The residue was purified by silica gel column chromatography (AcOEt:n-hexane=5:2) to give 4-ethoxy-3-(3-oxo-2-isopropyl-2,3-dihydropyridazin-6-yl)-2-phenylpyrazolo[1,5-a]pyridine (76.0 mg) as a solid.

mp: 157-158° C. (AcOEt-n-Hexane)

IR (KBr): 3097, 3055, 2979, 1657, 1589, 1545, 1284 cm⁻¹

¹H NMR (CDCl₃, δ): 1.12(6H,d,J=6.6 Hz), 1.30(3H,t,J=7.0 Hz), 4.08(2H,q,J=7.0 Hz), 5.26(1H,hept,J=6.6 Hz), 6.44(1H,d,J=7.6 Hz), 6.72(1H,dd,J=7.6, 6.9 Hz), 6.87(1H,d,J=9.5 Hz), 7.23-7.39(3H,m), 7.43(1H,d,J=9.5 Hz), 7.47-7.69(2H,m), 8.17 (1H,d,J=6.9 Hz)

APCI/MS: 375[M+H]⁺

### EXAMPLE 197

4-n-Propoxy-3-(3-oxo-2-isopropyl-2,3-dihydropyridazin-6-yl)-2-phenylpyrazolo [1,5-a]pyridine was prepared by similar procedure as that of Example 196.

mp: 176-177° C. (AcOEt-n-Hexane)

IR (KBr): 3097, 2979, 2935, 1655, 1591, 1545, 1284 cm⁻¹

¹H NMR (CDCl₃, δ): 0.85(3H,t,J=7.4 Hz), 1.13(6H,d,J=6.6 Hz), 1.55-1.78(2H,m), 3.97(2H,t,J=6.3 Hz), 5.27(1H,hept,J=6.6 Hz), 6.44(1H,d,J=7.6 Hz), 6.73(1H,dd,J=7.6, 6.9 Hz), 6.88(1H,d, J=9.5 Hz), 7.25-7.39(3H,m), 7.42(1H,d,J=9.5 Hz), 7.48-7.60(2H,m), 8.17(1H,dd,J=6.9, 0.8 Hz)

APCI/MS: 389[M+H]⁺

### EXAMPLE 198

4-(2-(Dimethylamino)ethoxy)-3-(3-oxo-2-isopropyl-2,3-dihydropyridazin-6-yl)-2-phenylpyrazolo[1,5-a]pyridine was prepared by similar procedure as that of Example 196.

mp: 137-138° C. (AcOEt-n-Hexane)

IR (KBr): 2979, 2763, 1658, 1585, 1282, 1097 cm⁻¹

¹H NMR (CDCl₃, δ): 1.10(6H,d,J=6.6 Hz), 2.20(6H,s), 2.59(2H,t, J=5.7 Hz), 4.13(2H,t,J=5.7 Hz), 5.25(1H,hept,J=6.6 Hz), 6.48(1H,d, J=7.6 Hz), 6.74(1H,dd,J=7.6, 7.0 Hz), 6.90(1H,d,J=9.5 Hz), 7.26-7.40(3H,m), 7.45-7.58(3H,m), 8.19(1H,d,J=7.0 Hz)

APCI/MS: 418[M+H]⁺

### EXAMPLE 199

4-(2-(4-Morpholinyl)ethoxy)-3-(3-oxo-2-isopropyl-2,3-dihydropyridazin-6-yl)-2-phenylpyrazolo[1,5-a]pyridine was prepared by similar procedure as that of Example 196.

IR (neat): 2962, 2931, 2856, 1658, 1589, 1547, 1286 cm⁻¹

¹HNMR (CDCl₃, δ): 1.09(6H,d,J=6.6 Hz), 2.35-2.48(4H,m), 2.66(2H, t,J=5.6 Hz), 3.55-3.73(4H,m), 4.16(2H,t,J=5.6 Hz), 5.25(1H,hept, J=6.6 Hz), 6.48(1H,d,J=7.6 Hz), 6.74(1H,t-like,J=7.2 Hz), 6.88(1H, d,J=9.5 Hz), 7.25-7.42(3H,m), 7.42-7.57(3H,m), 8.19(1H,d, J=6.8 Hz)

APCI/MS: 460[M+H]⁺

### EXAMPLES 200 TO 331

Desired Amide Derivatives of Examples 200 to 331, which are represented by the following formula (Ik) was obtained according to the following standard procedure.

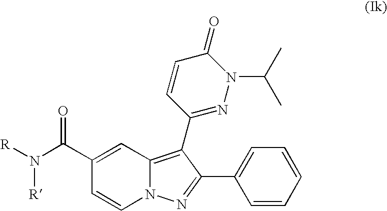

Wherein R and R′ are as defined as the following Table 3.

Standard procedure for the synthesis of Amide Derivatives (Ik)

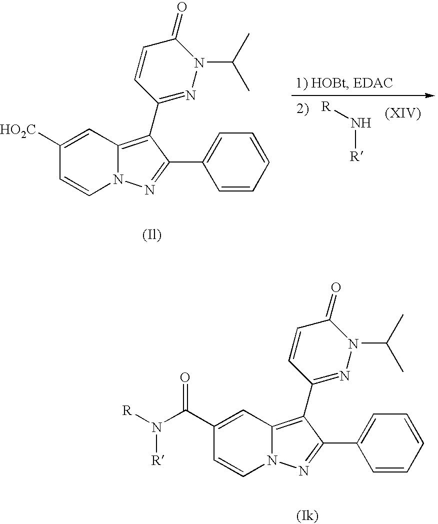

To a mixture of 3-(3-oxo-2-isopropyl-2,3-dihydropyridazin-6-yl)-2-phenylpyrazolo[1,5-a]pyridine-5-carboxylic acid (Il) (7.48 mg, 0.020 mmol), 1.0 M N,N-diisopropylethylamine in N-methyl-2-pyrrolidinone (NMP) (20 μl), 1.0 M solution of 1-hydroxybenzotriazole (HOBt) in NMP (22 μl) was added 1.0 M solution of 1-ethyl-3-(3′-dimethylaminopropyl)carbodiimide (EDAC) in NMP (24 μl) at ambient temperature. After stirring for 1 hour, the mixture was treated with 0.5 M solution of an amine(XIV) (defined as the following Table 3) in NMP (40 μl) at ambient temperature for 30 minutes, and then 60° C. for 90 minutes. The reaction mixture was purified by solid-phase extraction (SPE) on SPE column (Varian Nexus, 300 mg of sorbent) eluting with acetonitrile (400 μl) and then dichloroethane (400 μl). The fractions containing desired compound were concentrated and dried under reduced pressure to give desired compound (Ik). Purity was estimated by HPLC analysis (reverse phase C₁₈, 5μ, 4.6 mm×35 mm column, 254 nm, 0-80% CH₃CN (containing 0.05% HCO₂H)/H₂O (containing 0.05% HCO₂H), over 4 minutes, 2 ml/min).

Concerning each Example compound, the formula of amine

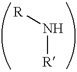

and MS spectrum of the desired compound (Ik) of the compound was shown in Table 3.

TABLE 3

| | Amines | | | |
|---|---|---|---|---|
| Example No. | 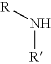 | MW | purity [%] | Mass [M + H]⁺ |
| 200 | 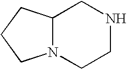 | 482.57 | 95 | 484 |
| 201 | 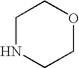 | 443.49 | 94 | 444 |
| 202 | 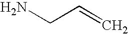 | 413.47 | 94 | 414 |
| 203 | 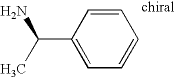 | 477.55 | 100 | 479 |
| 204 | 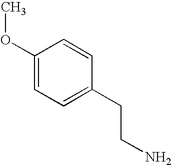 | 507.58 | 96 | 509 |
| 205 | 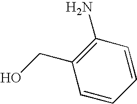 | 479.53 | 78 | 481 |
| 206 | 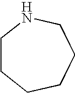 | 455.55 | 97 | 457 |
| 207 | 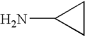 | 413.47 | 90 | 414 |
| 208 | 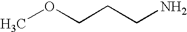 | 445.51 | 90 | 447 |
| 209 | 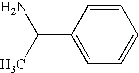 | 477.55 | 96 | 479 |
| 210 | 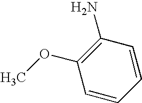 | 479.53 | 86 | 481 |
| 211 | 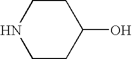 | 457.52 | 88 | 459 |
| 212 | 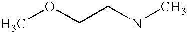 | 445.51 | 80 | 447 |
| 213 | 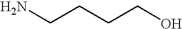 | 445.51 | 84 | 447 |
| 214 | 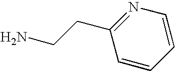 | 478.54 | 90 | 480 |
| 215 | 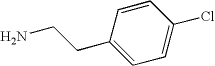 | 512.00 | 95 | 513 |
| 216 | 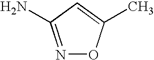 | 454.48 | 82 | 455 |
| 217 | 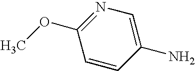 | 480.51 | 87 | 482 |
| 218 | 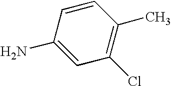 | 497.97 | 100 | 499 |
| 219 | 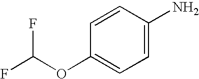 | 515.51 | 85 | 517 |
| 220 | 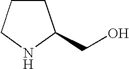 | 457.52 | 92 | 459 |
| 221 | 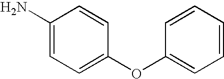 | 541.60 | 100 | 543 |
| 222 | 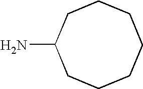 | 483.60 | 96 | 485 |
| 223 | 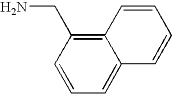 | 513.59 | 100 | 515 |
| 224 | 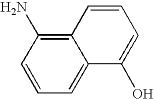 | 515.56 | 100 | 517 |
| 225 | 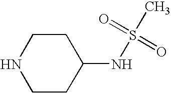 | 534.62 | 87 | 536 |
| 226 | 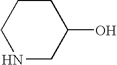 | 457.52 | 85 | 459 |
| 227 | 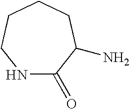 | 484.55 | 89 | 486 |
| 228 | 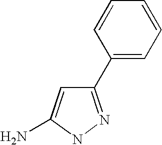 | 515.56 | 70 | 517 |
| 229 | 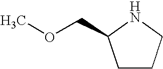 | 471.55 | 81 | 473 |
| 230 | 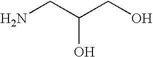 | 447.48 | 87 | 448 |
| 231 | 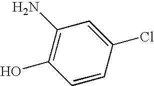 | 499.94 | 72 | 501 |
| 232 | 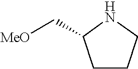 | 471.55 | 80 | 473 |
| 233 | 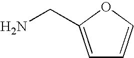 | 453.49 | 76 | 454 |
| 234 | 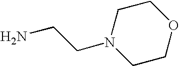 | 486.56 | 87 | 488 |
| 235 | 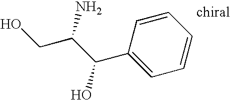 | 523.58 | 84 | 525 |
| 236 | 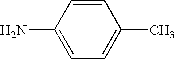 | 463.53 | 93 | 465 |
| 237 | 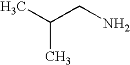 | 429.51 | 79 | 431 |
| 238 | 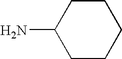 | 455.55 | 90 | 457 |
| 239 | 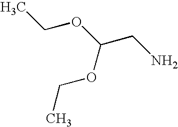 | 489.56 | 80 | 491 |
| 240 | 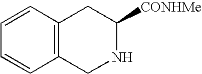 | 546.62 | 88 | 548 |
| 241 | 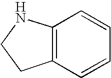 | 475.54 | 95 | 477 |
| 242 | 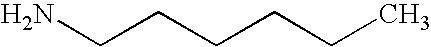 | 457.56 | 91 | 459 |
| 243 | 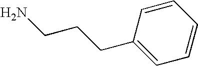 | 491.58 | 97 | 493 |
| 244 | 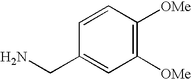 | 523.58 | 94 | 525 |
| 245 | 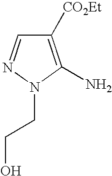 | 555.58 | 85 | 557 |
| 246 | 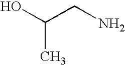 | 431.48 | 86 | 432 |
| 247 | 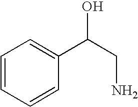 | 493.55 | 90 | 495 |
| 248 | 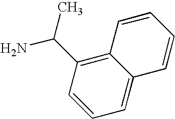 | 527.61 | 91 | 529 |
| 249 | 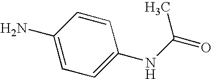 | 506.55 | 91 | 508 |
| 250 | 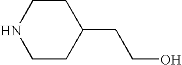 | 485.57 | 82 | 487 |
| 251 | 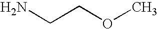 | 431.48 | 80 | 432 |
| 252 | 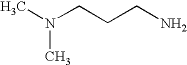 | 458.55 | 95 | 460 |
| 253 | 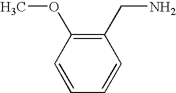 | 493.55 | 92 | 495 |
| 254 | 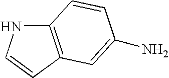 | 488.54 | 88 | 490 |
| 255 | 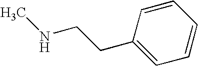 | 491.58 | 96 | 493 |
| 256 | 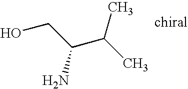 | 459.54 | 87 | 461 |
| 257 | 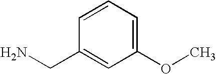 | 493.55 | 95 | 495 |
| 258 | 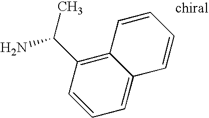 | 527.61 | 86 | 529 |
| 259 | 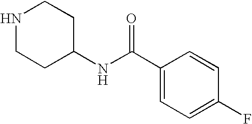 | 578.63 | 89 | 580 |
| 260 | 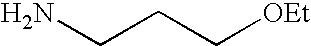 | 459.54 | 90 | 461 |
| 261 | 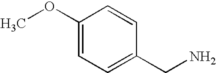 | 493.55 | 96 | 495 |
| 262 | 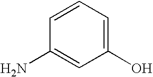 | 465.50 | 85 | 466 |
| 263 | 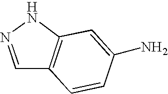 | 489.52 | 94 | 491 |
| 264 | 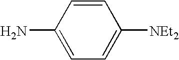 | 520.62 | 83 | 522 |
| 265 | 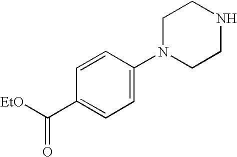 | 590.67 | 96 | 592 |
| 266 | 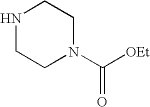 | 514.57 | 90 | 516 |
| 267 | 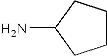 | 441.52 | 90 | 443 |
| 268 | 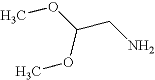 | 461.51 | 90 | 463 |
| 269 | 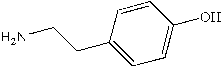 | 493.55 | 84 | 495 |
| 270 | 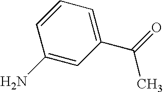 | 491.54 | 81 | 493 |
| 271 | 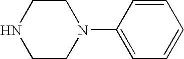 | 518.61 | 94 | 520 |
| 272 | 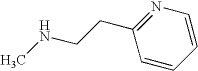 | 492.57 | 91 | 494 |
| 273 | 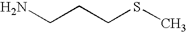 | 461.57 | 91 | 463 |
| 274 | 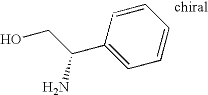 | 493.55 | 76 | 495 |
| 275 | 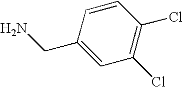 | 532.42 | 79 | 533 |
| 276 | 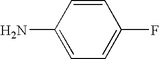 | 467.49 | 95 | 468 |
| 277 | 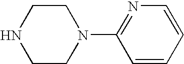 | 519.59 | 91 | 521 |
| 278 | 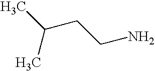 | 443.54 | 96 | 445 |
| 279 | 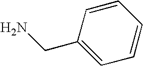 | 463.53 | 85 | 465 |
| 280 | 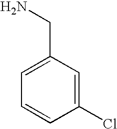 | 497.97 | 89 | 499 |
| 281 | 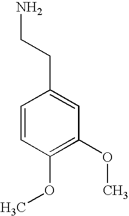 | 537.61 | 86 | 539 |
| 282 | 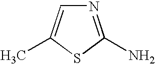 | 470.54 | 73 | 472 |
| 283 | 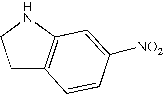 | 520.54 | 90 | 522 |
| 284 | 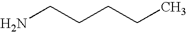 | 443.54 | 100 | 445 |
| 285 | 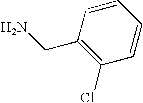 | 497.97 | 100 | 499 |
| 286 | 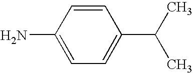 | 491.58 | 100 | 493 |
| 287 | 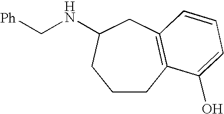 | 623.74 | 84 | 625 |
| 288 | 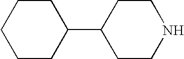 | 524.65 | 100 | 526 |
| 289 | 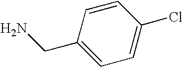 | 497.97 | 100 | 499 |
| 290 | 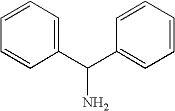 | 539.63 | 88 | 541 |
| 291 | 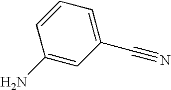 | 474.51 | 100 | 476 |
| 292 | 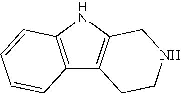 | 528.60 | 95 | 530 |
| 293 | 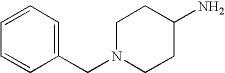 | 546.66 | 100 | 548 |
| 294 | 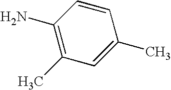 | 477.55 | 100 | 479 |
| 295 | 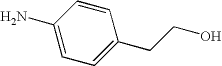 | 493.55 | 80 | 495 |
| 296 | 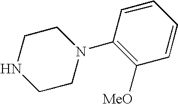 | 548.63 | 100 | 550 |
| 297 | 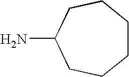 | 469.57 | 100 | 471 |
| 298 | 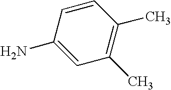 | 477.55 | 100 | 479 |
| 299 | 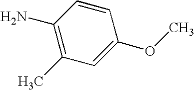 | 493.55 | 100 | 495 |
| 300 | 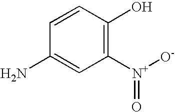 | 510.50 | 100 | 511 |
| 301 | 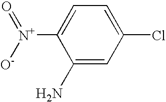 | 528.94 | 100 | 530 |
| 302 | 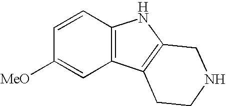 | 558.63 | 88 | 560 |
| 303 | 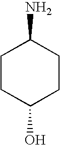 | 471.55 | 100 | 473 |
| 304 | 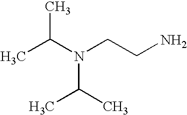 | 500.63 | 100 | 502 |
| 305 | 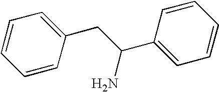 | 553.65 | 100 | 555 |
| 306 | 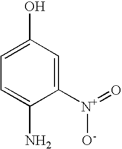 | 510.50 | 100 | 511 |
| 307 | 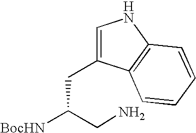 | 645.75 | 100 | 647 |
| 308 | 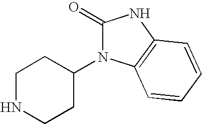 | 573.64 | 100 | 575 |
| 309 | 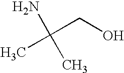 | 445.51 | 93 | 447 |
| 310 | 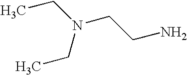 | 472.58 | 100 | 474 |
| 311 | 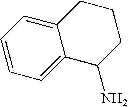 | 503.59 | 100 | 505 |
| 312 | 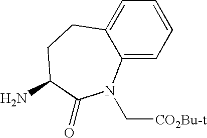 | 646.74 | 100 | 648 |
| 313 | 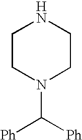 | 608.73 | 100 | 610 |
| 314 | 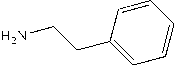 | 477.55 | 100 | 479 |
| 315 | 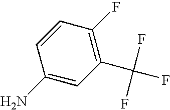 | 535.49 | 100 | 536 |
| 316 | 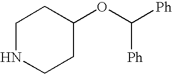 | 623.74 | 100 | 625 |
| 317 | 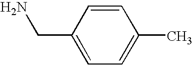 | 477.55 | 100 | 479 |
| 318 | 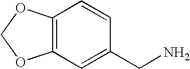 | 507.54 | 92 | 509 |
| 319 | 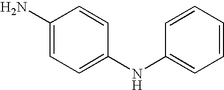 | 540.61 | 100 | 542 |
| 320 | 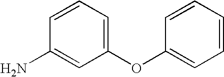 | 541.60 | 95 | 543 |
| 321 | 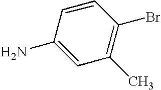 | 542.42 | 100 | 543 |
| 322 | 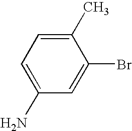 | 542.42 | 100 | 543 |
| 323 | 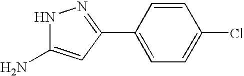 | 550.01 | 97 | 551 |
| 324 | 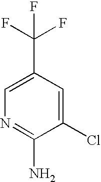 | 552.93 | 77 | 554 |
| 325 | 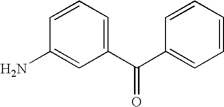 | 553.61 | 91 | 555 |
| 326 | 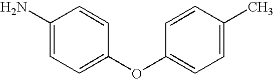 | 555.62 | 100 | 557 |
| 327 | 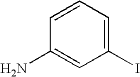 | 575.40 | 86 | 576 |
| 328 | 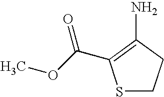 | 515.58 | 91 | 517 |
| 329 | 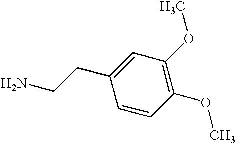 | 537.61 | 91 | 539 |
| 330 | 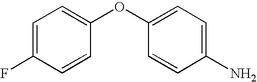 | 559.59 | 56 | 561 |
| 331 | 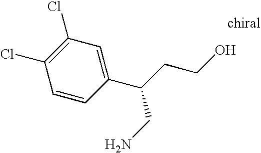 | 590.50 | 74 | 591 |

1. A pyrazolopyridine compound of the following formula (I). 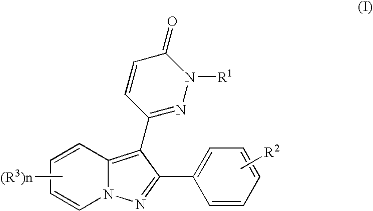
wherein
R¹ is hydrogen, lower alkyl optionally substituted by substituent(s), or cyclo(lower)alkyl which may be interrupted by an oxygen or nitrogen atom and optionally substituted by substituent(s);
R² is hydrogen, halogen or lower alkoxy;
R³ is a substituent; and
n is an integer from 1 to 4,
provided R³ may be different from each other when n is 2, 3 or 4,
or a salt thereof.

2. A compound of claim 1,
wherein
R¹ is hydrogen, lower alkyl optionally substituted by lower alkoxy, or cyclo(lower)alkyl which may be interrupted by an oxygen or nitrogen atom and optionally substituted by lower alkyl;
R³ is
(1) a group of the formula:
R⁴-A-O—
in which
A is lower alkylene, and
R⁴ is hydrogen;
cyclo(lower)alkyl;
aryl optionally substituted by lower alkoxy;
a group of the formula:
R⁵—(R⁶—)N—
wherein R⁵ and R⁶ are each independently
hydrogen, or
lower alkyl;
heterocyclic group optionally substituted by
oxo, lower alkyl or
lower alkoxy(lower)alkyl;
carboxy;
lower alkoxycarbonyl;
aryl(lower)alkoxycarbonyl;
lower alkanoyl;
a group of the formula:
R⁷—(R⁸—)N—CO—
wherein R⁷ and R⁸ are each independently
hydrogen;
lower alkyl optionally substituted by
lower alkoxy, N,N-di(lower)alkylamino or
heterocyclic group;
cyclo(lower)alkyl optionally substituted by hydroxy;
aryl optionally substituted by lower alkoxy; or
a group of the formula:
Het-CO—
wherein Het is N-containing heterocyclic group optionally substituted by
lower alkyl, lower alkanoyl, lower alkoxycarbonyl, N,N-di(lower)alkylcarbamoyl or aryl(lower)alkyl,
(2) a group of the formula:
R⁹—O—
in which
R⁹ is hydrogen;
aryl optionally substituted by lower alkanoylamino;
heterocyclic group optionally substituted by
lower alkyl, lower alkanoyl, lower alkoxycarbonyl, carbamoyl, N,N-di(lower)alkylcarbamoyl, aryl(lower)alkyl, lower alkoxy, halo(lower)alkyl or nitro; or
arylsulfonyl optionally substituted by
lower alkyl or lower alkoxy,
(3) a group of the formula:
R¹⁰—N(—R¹¹)—CO—
in which
R¹⁰ and R¹¹ are each independently
hydrogen;
cyclo(lower)alkyl;
heterocyclic group optionally substituted by lower alkyl;
lower alkyl optionally substituted by
hydroxy, lower alkoxy, aryl, aryloxy, N,N-di(lower)alkylamino or heterocyclic group; or
R¹⁰ and R¹¹ may be combined together with N atom to which they are attached to form N-containing heterocyclic group optionally substituted by
lower alkyl, aryl, lower alkanoyl or heterocyclic group,
(4) a group of the formula:
R¹²—N(—R¹³)—
in which
R¹² and R¹³ are each independently
hydrogen;
lower alkyl optionally substituted by lower alkoxy;
lower alkanoyl optionally substituted by aryl or halogen;
lower alkoxycarbonyl;
lower alkylsulfonyl; or
R¹² and R¹³ may be combined together with N atom to which they are attached to form N-containing heterocyclic group optionally substituted by
hydroxy, oxo, lower alkyl, lower alkoxy,
lower alkanoyl optionally substituted by
N,N-di(lower)alkylamino or aryl,
lower alkoxycarbonyl,
N,N-di(lower)alkylcarbamoyl,
lower alkylsulfonyl, arylsulfonyl, aryl,
aryl(lower)alkyl or heterocyclic group,
(5) a group of the formula:
R¹⁴-A′-
in which
A′ is lower alkynyl,
R¹⁴ is hydroxy; cyclo(lower)alkyl; or aryl, or
(6) carboxy, lower alkoxycarbonyl or cyano.

3. A compound of claim 2,
wherein
R¹ is hydrogen, lower alkyl optionally substituted by lower alkoxy, tetrahydrofuryl, tetrahydropyranyl or piperidinyl;
R³ is
(1) a group of the formula:
R⁴-A-O—
in which
A is lower alkylene, and
R⁴ is hydrogen;
cyclo(lower)alkyl;
phenyl optionally substituted by lower alkoxy;
a group of the formula:
R⁵—(R⁶—)N—
wherein R⁵ and R⁶ are each independently
hydrogen or lower alkyl;
aziridinyl, pyrrolidinyl, piperidinyl, morpholinyl, pyridyl or isoindolyl, each of which is optionally substituted by
oxo, lower alkyl or lower alkoxy(lower)alkyl;
carboxy;
lower alkoxycarbonyl;
phenyl(lower)alkoxycarbonyl;
lower alkanoyl;
a group of the formula:
R⁷—(R⁸—)N—CO—
wherein R⁷ and R⁸ are each independently
hydrogen;
lower alkyl optionally substituted by
lower alkoxy, N,N-di(lower)alkylamino or pyridyl;
cyclo(lower)alkyl optionally substituted by hydroxy;
phenyl optionally substituted by lower alkoxy; or
a group of the formula:
Het-CO—
wherein Het is pyrrolidinyl, piperidinyl, piperazinyl or morpholinyl, each of which is optionally substituted by
lower alkyl, lower alkanoyl, lower alkoxycarbonyl, N,N-di(lower)alkylcarbamoyl, phenyl(lower)alkyl,
(2) a group of the formula:
R⁹—O—
in which
R⁹ is hydrogen;
phenyl optionally substituted by lower alkanoylamino;
piperidinyl, tetrahydropyranyl or pyridinyl, each of which is optionally substituted by
lower alkyl, lower alkanoyl, lower alkoxycarbonyl, carbamoyl, N,N-di(lower)alkylcarbamoyl, phenyl(lower)alkyl, lower alkoxy, halo(lower)alkyl or nitro;
phenylsulfonyl optionally substituted by
lower alkyl or lower alkoxy,
(3) a group of the formula:
R¹⁰—N(—R¹¹)—CO—
in which
R¹⁰ and R¹¹ are each independently
hydrogen;
cyclo(lower)alkyl;
thiazolyl optionally substituted by lower alkyl;
lower alkyl optionally substituted by
hydroxy, lower alkoxy, phenyl, phenoxy, N,N-di(lower)alkylamino, pyrrolidinyl or pyridinyl; or
R¹⁰ and R¹¹ may be combined together with N atom to which they are attached to form pyrrolidinyl, piperidinyl, hexahydroazepinyl, piperazinyl or morpholinyl, each of which is optionally substituted by lower alkyl, phenyl, lower alkanoyl or pyridinyl,
(4) a group of the formula:
R¹²—N(—R¹³)—
in which
R¹² and R¹³ are each independently
hydrogen;
lower alkyl optionally substituted by lower alkoxy;
lower alkanoyl optionally substituted by phenyl or halogen;
lower alkoxycarbonyl;
lower alkylsulfonyl; or
R¹² and R¹³ may be combined together with N atom to which they are attached to form pyrrolidinyl, piperidinyl, piperazinyl or morpholinyl, each of which is optionally substituted by
hydroxy, oxo, lower alkyl, lower alkoxy,
lower alkanoyl optionally substituted by
N,N-di(lower)alkylamino or phenyl,
lower alkoxycarbonyl,
N,N-di(lower)alkylcarbamoyl
lower alkylsulfonyl, phenylsulfonyl, phenyl,
phenyl(lower)alkyl, pyridinyl or pyrimidinyl,
(5) a group of the formula:
R¹⁴-A′-
in which
A′ is lower alkynyl,
R¹⁴ is hydroxy; cyclo(lower)alkyl; or phenyl, or
(6) carboxy, lower alkoxycarbonyl or cyano.

4. A compound of claim 3,
wherein
R¹ is (C1-C4)alkyl,
R² is hydrogen,
R³ is (C1-C4)alkoxy, and
n is 1.

5. A process for preparing the pyrazolopyridine compound of the following formula (I). 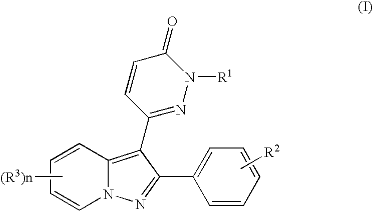
wherein R¹, R², R³ and n are each as defined in claim 1,
or a salt thereof, which comprises
(1) hydrolyzing a compound of the formula (II): 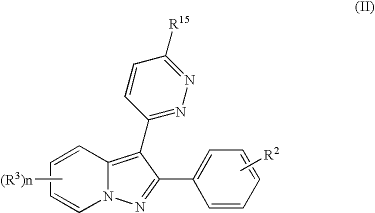
wherein
R², R³ and n are each as defined above, and
R¹⁵ is arylsulfonyl optionally substituted by substituent(s), di(lower)alkylamino, lower alkoxy, lower alkylthio, or acyloxy;
or a salt thereof,
to give a compound of the formula (Ia): 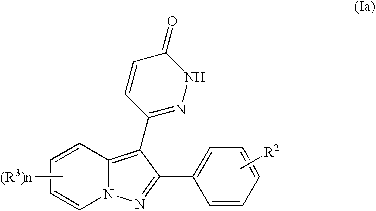
wherein R², R³ and n are each as defined above
or a salt thereof,
(2) reacting a compound of the formula (Ia) or a salt thereof, with a compound of the formula (III):
R^{1a}—Y (III)
wherein R^{1a} is lower alkyl or cyclo(lower)alkyl which may be interrupted by an oxygen atom, and
Y is a leaving group;
or a salt thereof,
to give a compound of the formula (Ib): 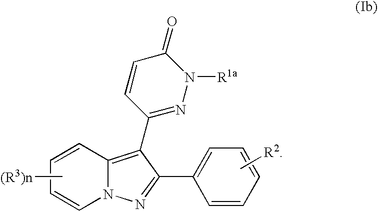
wherein R^{1a}, R², R³ and n are as defined above
or a salt thereof,
(3) eliminating of alkyl group of a compound of the formula (Ic): 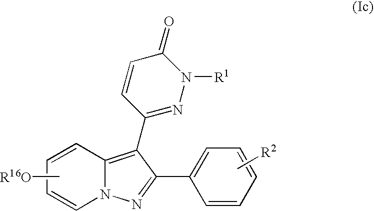
wherein
R¹ and R² are as defined above; and
R¹⁶ is lower alkyl, or a salt thereof,
to give a compound of a formula (Id): 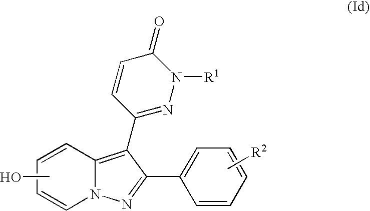
wherein R¹ and R² are as defined above,
or a salt thereof, or
(4) reacting a compound of the formula (Id): 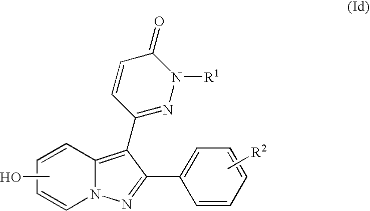
wherein R¹ and R² are as defined above or a salt thereof,
with a compound of the formula (IV):
R¹⁷—Y (IV)
wherein R¹⁷ is a substituent selected from the group consisting of a group of the formula: -A-R⁴ and a group of the formula: —R⁹
[wherein A is as defined above, and R⁴ and R⁹ are each as defined in claim 2], and
Y is a leaving group,
or a salt thereof,
to give a compound of the formula (Ie): 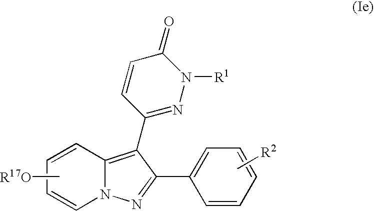
wherein R¹, R² and R¹⁷ are as defined above
or a salt thereof.
(5) subjecting a compound of the formula (If): 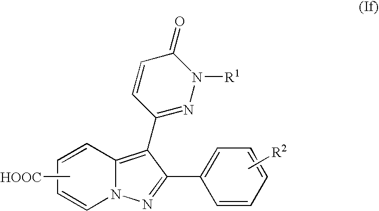
wherein R¹ and R² are as defined above, or a salt thereof, to acylation reaction with an amine of the formula (V):
R¹⁰—NH—R¹¹ (V)
to give a compound of the formula (Ig): 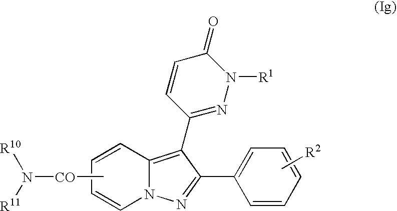
wherein R¹ and R² are as defined above, and
R¹⁰ and R¹¹ are each as defined in claim 2,
or a salt thereof,
(6) subjecting a compound of the formula (Ih): 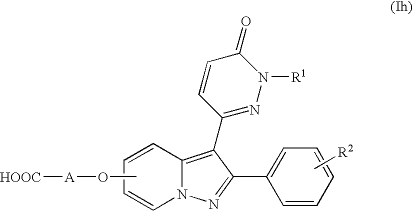
wherein R¹ and R² are as defined above, and A is lower alkylene,
or a salt thereof,
to acylation reaction with an amine of the formula (VI):
R⁷—NH—R⁸ (VI)
to give a compound of the formula (Ii): 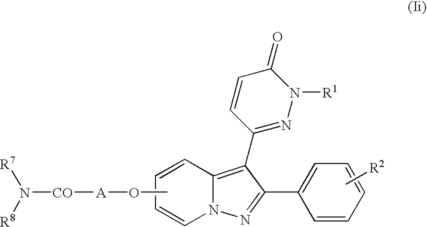
wherein R¹, R² and A are as defined above, and
R⁷ and R⁸ are each as defined in claim 2,
or a salt thereof,
(7) reacting a compound of the formula (VII): 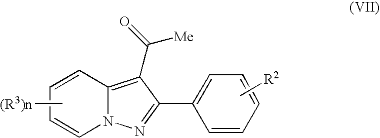
wherein
R², R³ and n are as defined above,
or a salt thereof,
with hydrazine and glyoxylic acid,
to give a compound of the formula (Ia): 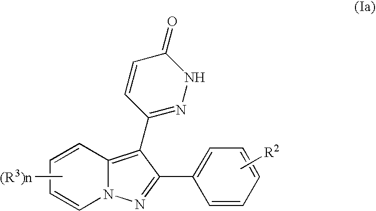
wherein
R², R³ and n are as defined above
or a salt thereof.

6. A pharmaceutical composition comprising the compound of claim 1 or a pharmaceutically acceptable salt thereof in admixture with a pharmaceutically acceptable carrier.

7. A process for preparing a pharmaceutical composition which comprises admixing the compound of claim 1 or a pharmaceutically acceptable salt thereof with a pharmaceutically acceptable carrier.

8. A method for preventing or treating a disease resulting from a stimulation of adenosine A₁ and/or A₂ receptor in a human being or an animal, which comprises administering the compound of claim 1 or a pharmaceutically acceptable salt thereof to a human being or an animal.

9. A method for preventing or treating a disease on which an adenosine antagonist is therapeutically effective, which comprises administering the compound of claim 1 or a pharmaceutically acceptable salt thereof to a human being or an animal.

10. A method for preventing or treating a disease selected from the group consisting of depression, dementia, Parkinson's disease, anxiety, pain, cerebrovascular disease, heart failure, hypertension, circulatory insufficiency, post-resuscitation, asystole, bradyarrhythmia, electro-mechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack and angina pectoris, which comprises administering the compound of claim 1 or a pharmaceutically acceptable salt thereof to a human being or an animal.

11. A method for preventing or treating a disease selected from the group consisting of Parkinson's disease and symptoms associating therewith, which comprises administering the compound of claim 1 or a pharmaceutically acceptable salt thereof to a human being or an animal.

12. A compound of claim 1 or a pharmaceutically acceptable salt thereof for use as a medicament.

13. A compound of claim 1 or a pharmaceutically acceptable salt thereof for use as an adenosine antagonist.

14. A compound of claim 1 or a pharmaceutically acceptable salt thereof for use as an adenosine A₁ receptor and A₂ receptor dual antagonist.

15. Use of the compound of claim 1 or a pharmaceutically acceptable salt thereof for the production of a pharmaceutical composition for the therapy and/or prevention of a disease resulting from a stimulation of adenosine A₁ and/or A₂ receptor.

16. Use of the compound of claim 1 or a pharmaceutically acceptable salt thereof for the production of a pharmaceutical composition for the therapy of a disease on which an adenosine antagonist is therapeutically effective.

17. A method for evaluation of adenosine antagonism, which comprises use of a compound of claim 1 or a pharmaceutically acceptable salt thereof.
